(19) United States
(12) Patent Application Publication
(10) Pub. No.: US 2026/0297572 A1
(43) Pub. Date: Oct. 1, 2026

(54) Nucleic Acid Antisense Oligomer Readthrough of Nonsense Codons

(71) Applicant: University of Massachusetts, Westborough, MA (US)

(72) Inventors: Denis Susorov, Worcester, MA (US); Andrei A. Korostelev, Shrewsbury, MA (US); Zahra Seraj, Worcester, MA (US); Anastasia Khvorova, Westborough, MA (US); Dimas Echeverria Moreno, Natick, MA (US)

(21) Appl. No.: 18/998,232

(22) PCT Filed: Jul. 24, 2023
(86) PCT No.: PCT/US2023/028459

§ 371 Date: Jan. 24, 2025

Related U.S. Application Data
(60) Provisional application No. 63/391,929, filed on Jul. 25, 2022.

(51) Int. Cl. C12N 15/113

(52) U.S. Cl. CPC C12N 15/113; C12N 2310/11; C12N 2310/3125; C12N 2310/315; C12N 2310/321; C12N 2310/322; C12N 2310/3233

(57) ABSTRACT

This invention is related to the field of genetic engineering. In particular, it is related to compositions and methods to treat genetically-based diseases and disorder or diseases that are caused by the translation of non-functional proteins from an mRNA with a nonsense (premature) stop codon. Nonsense stop codon readthrough results in a full-length protein and restores protein function. For example, a combination of a suppressor transfer ribonucleic acid (stRNA) and nucleic acid antisense oligomers are contemplated that promote translation readthrough of mRNAs with nonsense stop codons.

### STATEMENT OF GOVERNMENTAL SUPPORT

This invention was made with government support under GM127094 awarded by The National Institutes of Health. The government has certain rights in the invention.

### FIELD OF THE INVENTION

This invention is related to the field of genetic engineering. In particular, it is related to compositions and methods to treat genetically-based diseases and disorder or diseases that are caused by the translation of non-functional proteins from an mRNA with a premature (nonsense) stop codon. Premature stop codon readthrough results in a full-length protein and restores protein function. For example, a combination of a suppressor transfer ribonucleic acid (stRNA) and nucleic acid antisense oligomers are contemplated that promote translation readthrough of mRNA premature (nonsense) stop codons.

### BACKGROUND

Mutations resulting in premature nonsense (i.e., stop) codons lead to more than 10% of human genetic diseases. Stop codons normally result in the physical release of proteins from the ribosome. Thus, a premature stop codon results in the translation of a short (e.g. truncated), often non-functional protein, resulting in disease.

Most tested drugs (e.g. aminoglycosides, such as G418) can provide global readthrough of nonsense or stop codons but induce miscoding for many cellular mRNAs, thereby resulting in a clinically unacceptable level of toxicity.

What is needed in the art are compositions and methods to increase readthrough of a premature stop codon in a specific mRNA molecule.

### SUMMARY OF THE INVENTION

This invention is related to the field of genetic engineering. In particular, it is related to compositions and methods to treat genetically-based diseases and disorder or diseases that are caused by the translation of non-functional proteins from an mRNA with a premature (nonsense) stop codon. Premature stop codon readthrough results in a full-length protein and restores protein function. For example, a combination of a suppressor transfer ribonucleic acid (stRNA) and nucleic acid antisense oligomers are contemplated that promote translation readthrough of mRNA premature (nonsense) stop codons.

In one embodiment, the present invention contemplates a method, comprising: a) providing; i) a patient comprising a messenger ribonucleic acid (mRNA) molecule having a nonsense stop codon and exhibits at least one symptom of a genetic disorder or disease; and ii) a pharmaceutically acceptable composition comprising a nucleic acid antisense oligomer that is at least partially complementary to the mRNA starting between a +4-+9 nucleotide position downstream of the first nucleotide of the nonsense stop codon; and b) administering the pharmaceutically acceptable composition to the patient such that said at least one symptom of said genetic disorder or disease is reduced. In one embodiment, the pharmaceutical composition further comprises a suppressor tRNA (stRNA) that is complementary to the nonsense stop codon. In one embodiment, the stRNA is aminoacylated. In one embodiment, the stRNA is ser-tRNA^{UGA}.

In one embodiment, the nucleic acid antisense oligomer includes, but is not limited to, SEQ ID NO:'s 1-16. In one embodiment, the nucleic acid antisense oligomer includes, but is not limited to, SEQ ID NOs: 712-730. In one embodiment, the nucleic acid antisense oligomer comprises, or consists of, contemplates a nucleic acid antisense oligomer comprising, or consisting of, a plurality of nucleic acids having the sequence of:

5′-A₂₀-A₁₉-A₁₈-A₁₇-A₁₆-A₁₅-A₁₄-A₁₃-A₁₂-A₁₁-A₁₀-A₉₋
A₈₋A₇₋A₆-A₅₋A₄₋A₃₋A₂₋A₁₋3′

wherein, A₂₀ is any nucleic acid, A₁₉ is any nucleic acid, A₁₈ is any nucleic acid, A₁₇ is any nucleic acid, A₁₆ is T, A₁₅ is any nucleic acid, A₁₄ is any nucleic acid, A₁₃ is any nucleic acid, A₁₂ is any nucleic acid, A₁₁ is any nucleic acid, A₁₀ is G, A₉ is any nucleic acid, A₈ is any nucleic acid, A₇ is A, A₆ is any nucleic acid, A₅ is any nucleic acid, A₄ is C, A₃ is any nucleic acid, A₂ is any nucleic acid and A₁ is C. In one embodiment, the nucleic acid antisense oligomer includes, but is not limited to SEQ ID NO:'s 36-54. In one embodiment, the nucleic acid antisense oligomer comprising, or consisting of, a plurality of nucleic acids having the sequence of:

5′-B₂₀-B₁₉-B₁₈-B₁₇-B₁₆-B₁₅-B₁₄-B₁₃-B₁₂-B₁₁-B₁₀-B₉₋
B₈₋B₇₋B₆₋B₅₋B₄₋B₃₋B₂₋B₁₋3′

wherein, B₂₀ is any nucleic acid, B₁₉ is any nucleic acid, B₁₈ is any nucleic acid, B₁₇ is any nucleic acid, B₁₆ is T, B₁₅ is any nucleic acid, B₁₄ is any nucleic acid, B₁₃ is any nucleic acid, B₁₂ is any nucleic acid, B₁₁ is any nucleic acid, B₁₀ is any nucleic acid, B₉ is any nucleic acid, B₈ is any nucleic acid, B₇ is A, B₆ is any nucleic acid, B₅ is any nucleic acid, B₄ is C, B₃ is any nucleic acid, B₂ is any nucleic acid and B₁ is C. In one embodiment, the nucleic acid antisense oligomer includes, but is not limited to, SEQ ID NO:'s 120-154. In one embodiment, the nucleic acid antisense oligomer comprising, or consisting of, a plurality of nucleic acids having the sequence of:

5′-C₂₀-C₁₉-C₁₈-C₁₇-C₁₆-C₁₅-C₁₄-C₁₃-C₁₂-C₁₁-C₁₀-C₉₋
C₈₋C₇₋C₆₋C₅₋C₄₋C₃₋C₂₋C₁₋3′

wherein, C₂₀ is any nucleic acid, C₁₉ is any nucleic acid, C₁₈ is any nucleic acid, C₁₇ is any nucleic acid, C₁₆ is any nucleic acid, C₁₅ is any nucleic acid, C₁₄ is any nucleic acid, C₁ is any nucleic acid, C₁₂ is any nucleic acid, C₁₁ is any nucleic acid, C₁₀ is G, C₉ is any nucleic acid, C₈ is any nucleic acid, C₇ is A, C₆ is any nucleic acid, C₅ is any nucleic acid, C₄ is C, C₃ is any nucleic acid, C₂ is any nucleic acid and C₁ is C. In one embodiment, the nucleic acid antisense oligomer includes, but is not limited to, SEQ ID NO:'s 242-298. In one embodiment, the nucleic acid antisense oligomer comprising, or consisting of, a plurality of nucleic acids having the sequence of:

5′-D₂₀-D₁₉-D₁₈-D₁₇-D₁₆-D₁₅-D₁₄-D₁₃-D₁₂-D₁₁-D₁₀-D₉₋
D₈₋D₇₋D₆₋D₅₋D₄₋D₃₋D₂₋D₁₋3′

wherein, D₂₀ is any nucleic acid, D₁₉ is any nucleic acid, D₁₈ is any nucleic acid, D₁₇ is any nucleic acid, D₁₆ is any nucleic acid, D₁₅ is any nucleic acid, D₁₄ is any nucleic acid, D₁₃ is any nucleic acid, D₁₂ is any nucleic acid, D₁₁ is any nucleic acid, D₁₀ is any nucleic acid, D₉ is any nucleic acid, D₈ is any nucleic acid, D₇ is A, D₆ is any nucleic acid, D₈ is any nucleic acid, D₄ is C, D₃ is any nucleic acid, D₂ is any nucleic acid and D₁ is C. In one embodiment, the nucleic acid antisense oligomer includes, but is not limited to, SEQ ID NO:'s 496-692. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the mRNA encodes a protein. In one embodiment, the genetic disease is caused by a truncated expression of said protein. In one embodiment, the at least one symptom is reduced by a full length expression of said protein. In one embodiment, the genetic disorder or disease includes, but is not limited to, Duchenne muscular dystrophy, non-spherocytic hemolytic anemia, inherited retinal diseases (IRD), ataxia-telangiectasia, Miyoshi myopathy, limb-girdle muscular dystrophy, distal anterior compartment myopathy, recessive retinitis pigmentosa, breast cancer, ovarian cancer, retinitis pigmentosa, Alagille syndrome, Stickler syndrome, choroideremia, bull's-eye maculopathy, familial breast cancer, pancreatic cancer, neurofibromatosis type 1 Usher syndrome, muscular dystrophy and cystic fibrosis. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the nonsense stop codon comprises UAA. In one embodiment, the nonsense stop codon comprises UAG. In one embodiment the nonsense stop codon comprises UGA. In one embodiment, the nonsense stop codon is UGAC. In one embodiment, the nonsense stop codon is UGAG. In one embodiment, the nonsense stop codon is UGAA. In one embodiment, the nonsense stop codon is UGAU. In one embodiment, the nucleic acid antisense oligomer is single stranded. In one embodiment, the nucleic acid antisense oligomer is double stranded.

In one embodiment, the present invention contemplates a method, comprising: a) providing; i) a patient comprising a messenger ribonucleic acid (mRNA) molecule having a nonsense stop codon and exhibiting at least one symptom of a genetic disorder or disease; and ii) a pharmaceutically acceptable composition comprising: A) a nucleic acid antisense oligomer that is at least partially complementary to the mRNA starting between a +4-+9 nucleotide position downstream of the first nucleotide of the nonsense stop codon and B) a suppressor transfer ribonucleic acid (stRNA); and b) administering the pharmaceutically acceptable composition to the patient such that said at least one symptom of said genetic disorder or disease is reduced. In one embodiment, the stRNA is complementary to the nonsense stop codon. In one embodiment, the stRNA is aminoacylated. In one embodiment, the stRNA is ser-tRNA^{UGA}. In one embodiment, the nucleic acid antisense oligomer includes, but is not limited to, SEQ ID NO:'s 1-16. In one embodiment, the nucleic acid antisense oligomer includes, but is not limited to, SEQ ID NOs: 712-730. In one embodiment, the nucleic acid antisense oligomer comprises, or consists of, contemplates a nucleic acid antisense oligomer comprising, or consisting of, a plurality of nucleic acids having the sequence of:

5′-A₂₀-A₁₉-A₁₈-A₁₇-A₁₆-A₁₅-A₁₄-A₁₃-A₁₂-A₁₁-A₁₀-A₉₋
A₈₋A₇₋A₆₋A₅₋A₄₋A₃₋A₂₋A₁₋3′

wherein, A₂₀ is any nucleic acid, A₁₉ is any nucleic acid, A₁₈ is any nucleic acid, A₁₇ is any nucleic acid, A₁₆ is T, A₁₅ is any nucleic acid, A₁₄ is any nucleic acid, A₁₃ is any nucleic acid, A₁₂ is any nucleic acid, A₁₁ is any nucleic acid, A₁₀ is G, A₉ is any nucleic acid, A₈ is any nucleic acid, A₇ is A, A₆ is any nucleic acid, A₅ is any nucleic acid, A₄ is C, A₃ is any nucleic acid, A₂ is any nucleic acid and A₁ is C. In one embodiment, the nucleic acid antisense oligomer includes, but is not limited to, SEQ ID NO:'s 36-54. In one embodiment, the nucleic acid antisense oligomer comprising, or consisting of, a plurality of nucleic acids having the sequence of:

5′-B₂₀-B₁₉-B₁₈-B₁₇-B₁₆-B₁₅-B₁₄-B₁₃-B₁₂-B₁₁-B₁₀-B₉₋
B₈₋B₇₋B₆₋B₅₋B₄₋B₃₋B₂₋B₁₋3′

wherein, B₂₀ is any nucleic acid, B₁₉ is any nucleic acid, B₁₈ is any nucleic acid, B₁₇ is any nucleic acid, B₁₆ is T, B₁₅ is any nucleic acid, B₁₄ is any nucleic acid, B₁₃ is any nucleic acid, B₁₂ is any nucleic acid, Bu is any nucleic acid, B₁₀ is any nucleic acid, B₉ is any nucleic acid, B₈ is any nucleic acid, B₇ is A, B₆ is any nucleic acid, B₅ is any nucleic acid, B₄ is C, B₃ is any nucleic acid, B₂ is any nucleic acid and B₁ is C. In one embodiment, the nucleic acid antisense oligomer includes, but is not limited to, SEQ ID NO:'s 120-154. In one embodiment, the nucleic acid antisense oligomer comprising, or consisting of, a plurality of nucleic acids having the sequence of:

5′-C₂₀-C₁₉-C₁₈-C₁₇-C₁₆-C₁₅-C₁₄-C₁₃-C₁₂-C₁₁-C₁₀-C₉₋
C₈₋C₇₋C₆₋C₅₋C₄₋C₃₋C₂₋C₁₋3′

wherein, C₂₀ is any nucleic acid, C₁₉ is any nucleic acid, C₁₈ is any nucleic acid, C₁₇ is any nucleic acid, C₁₆ is any nucleic acid, C₁₅ is any nucleic acid, C₁₄ is any nucleic acid, C₁₃ is any nucleic acid, C₁₂ is any nucleic acid, C₁₁ is any nucleic acid, C₁₀ is G, C₉ is any nucleic acid, C₈ is any nucleic acid, C₇ is A, C₆ is any nucleic acid, C₅ is any nucleic acid, C₄ is C, C₃ is any nucleic acid, C₂ is any nucleic acid and C₁ is C. In one embodiment, the nucleic acid antisense oligomer includes, but is not limited to, SEQ ID NO:'s 242-298. In one embodiment, the nucleic acid antisense oligomer comprising, or consisting of, a plurality of nucleic acids having the sequence of:

5′-D₂₀-D₁₉-D₁₈-D₁₇-D₁₆-D₁₅-D₁₄-D₁₃-D₁₂-D₁₁-D₁₀-D₉₋
D₈₋D₇₋D₆₋D₅₋D₄₋D₃₋D₂₋D₁₋3′

wherein, D₂₀ is any nucleic acid, D₁₉ is any nucleic acid, D₁₈ is any nucleic acid, D₁₇ is any nucleic acid, D₁₆ is any nucleic acid, D₁₅ is any nucleic acid, D₁₄ is any nucleic acid, D₁₃ is any nucleic acid, D₁₂ is any nucleic acid, D₁₁ is any nucleic acid, D₁₀ is any nucleic acid, D₉ is any nucleic acid, D₈ is any nucleic acid, D₇ is A, D₆ is any nucleic acid, D₅ is any nucleic acid, D₄ is C, D₃ is any nucleic acid, D₂ is any nucleic acid and D₁ is C. In one embodiment, the nucleic acid antisense oligomer includes, but is not limited to, SEQ ID NO:'s 496-692. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN),a 2′-O-methyl (Ome), a 2′-methoxyethyl (Moe) or a mismatched nucleotide. In one embodiment, the mRNA encodes a protein. In one embodiment, the genetic disease is caused by a truncated expression of said protein. In one embodiment, the at least one symptom is reduced by a full length expression of said protein. In one embodiment, the genetic disorder or disease includes, but is not limited to, Duchenne muscular dystrophy, non-spherocytic hemolytic anemia, inherited retinal diseases (IRD), ataxia-telangiectasia, Miyoshi myopathy, limb-girdle muscular dystrophy, distal anterior compartment myopathy, recessive retinitis pigmentosa, breast cancer, ovarian cancer, retinitis pigmentosa, Alagille syndrome, Stickler syndrome, choroideremia, bull's-eye maculopathy, familial breast cancer, pancreatic cancer, neurofibromatosis type 1 Usher syndrome, muscular dystrophy and cystic fibrosis. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the nonsense stop codon comprises UAA. In one embodiment, the nonsense stop codon comprises UAG. In one embodiment the nonsense stop codon comprises UGA. In one embodiment, the nonsense stop codon is UGAC. In one embodiment, the nonsense stop codon is UGAG. In one embodiment, the nonsense stop codon is UGAA. In one embodiment, the nonsense stop codon is UGAU. In one embodiment, the nucleic acid antisense oligomer is single stranded. In one embodiment, the nucleic acid antisense oligomer is double stranded. In one embodiment, the pharmaceutically acceptable composition is a adeno-associated virus. In one embodiment, the pharmaceutically acceptable composition is selected from the group consisting of a microparticle, a nanoparticle and a liposome. In one embodiment, the pharmaceutically acceptable composition is selected from the group consisting of a tablet, a capsule and a gel.

In one embodiment, the present invention contemplates a nucleic acid antisense oligomer comprising, or consisting of, a plurality of nucleic acids having the sequence of:

5′-A₂₀-A₁₉-A₁₈-A₁₇-A₁₆-A₁₅-A₁₄-A₁₃-A₁₂-A₁₁-A₁₀-A₉₋
A₈₋A₇₋A₆₋A₅₋A₄₋A₃₋A₂₋A₁₋3′

wherein, A₂₀ is any nucleic acid, A₁₉ is any nucleic acid, A₁₈ is any nucleic acid, A₁₇ is any nucleic acid, A₁₆ is T, A₁₅ is any nucleic acid, A₁₄ is any nucleic acid, A₁₃ is any nucleic acid, A₁₂ is any nucleic acid, A₁₁ is any nucleic acid, A₁₀ is G, A₉ is any nucleic acid, A₈ is any nucleic acid, A₇ is A, A₆ is any nucleic acid, A₅ is any nucleic acid, A₄ is C, A₃ is any nucleic acid, A₂ is any nucleic acid and A₁ is C. In one embodiment, the nucleic acid antisense oligomer includes but is not limited to, SEQ ID NOs: 36-54. In one embodiment, the nucleic acid antisense oligomer comprises at least one nucleic acid comprising a chemical modification. In one embodiment, the chemical modification includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the nucleic acid antisense oligomer is single stranded. In one embodiment, the nucleic acid antisense oligomer is double stranded.

In one embodiment, the present invention contemplates a composition comprising: i) contemplates a nucleic acid antisense oligomer comprising, or consisting of, a plurality of nucleic acids having the sequence of:

5′-A₂₀-A₁₉-A₁₈-A₁₇-A₁₆-A₁₅-A₁₄-A₁₃-A₁₂-A₁₁-A₁₀-A₉₋
A₈₋A₇₋A₆₋A₅₋A₄₋A₃₋A₂₋A₁₋3′

wherein, A₂₀ is any nucleic acid, A₁₉ is any nucleic acid, A₁₈ is any nucleic acid, A₁₇ is any nucleic acid, A₁₆ is T, A₁₅ is any nucleic acid, A₁₄ is any nucleic acid, A₁₃ is any nucleic acid, A₁₂ is any nucleic acid, A₁₁ is any nucleic acid, A₁₀ is G, A₉ is any nucleic acid, A₈ is any nucleic acid, A₇ is A, A₆ is any nucleic acid, A₅ is any nucleic acid, A₄ is C, A₃ is any nucleic acid, A₂ is any nucleic acid and A₁ is C; and ii) a suppressor transfer ribonucleic acid (stRNA). In one embodiment, the nucleic acid antisense oligomer includes, but is not limited to, SEQ ID NOs: 36-54. In one embodiment, the stRNA is aminoacylated. In one embodiment, the stRNA is ser-tRNA^{UGA}. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the nucleic acid antisense oligomer is single stranded. In one embodiment, the nucleic acid antisense oligomer is double stranded.

In one embodiment, the present invention contemplates a nucleic acid antisense oligomer comprising, or consisting of, a plurality of nucleic acids having the sequence of:

5′-B₂₀-B₁₉-B₁₈-B₁₇-B₁₆-B₁₅-B₁₄-B₁₃-B₁₂-B₁₁-B₁₀-B₉₋
B₈₋B₇₋B₆₋B₅₋B₄₋B₃₋B₂₋B₁₋3′

wherein, B₂₀ is any nucleic acid, B₁₉ is any nucleic acid, B₁₈ is any nucleic acid, B₁₇ is any nucleic acid, B₁₆ is T, B₁₅ is any nucleic acid, B₁₄ is any nucleic acid, B₁₃ is any nucleic acid, B₁₂ is any nucleic acid, Bu is any nucleic acid, B₁₀ is any nucleic acid, B₉ is any nucleic acid, B₈ is any nucleic acid, B₇ is A, B₆ is any nucleic acid, B₅ is any nucleic acid, B₄ is C, B₃ is any nucleic acid, B₂ is any nucleic acid and B₁ is C. In one embodiment, the nucleic acid antisense oligomer includes, but is not limited to, SEQ ID NOs: 120-184. In one embodiment, the nucleic acid antisense oligomer comprises at least one nucleic acid comprising a chemical modification. In one embodiment, the chemical modification includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the nucleic acid antisense oligomer is single stranded. In one embodiment, the nucleic acid antisense oligomer is double stranded.

In one embodiment, the present invention contemplates a composition comprising: i) a nucleic acid antisense oligomer comprising, or consisting of, a plurality of nucleic acids having the sequence of:

5′-B₂₀-B₁₉-B₁₈-B₁₇-B₁₆-B₁₅-B₁₄-B₁₃-B₁₂-B₁₁-B₁₀-B₉₋
B₈₋B₇₋B₆₋B₅₋B₄₋B₃₋B₂₋B₁₋3′

wherein, B₂₀ is any nucleic acid, B₁₉ is any nucleic acid, B₁₈ is any nucleic acid, B₁₇ is any nucleic acid, B₁₆ is T, B₁₅ is any nucleic acid, B₁₄ is any nucleic acid, B₁₃ is any nucleic acid, B₁₂ is any nucleic acid, B₁₁ is any nucleic acid, B₁₀ is any nucleic acid, B₉ is any nucleic acid, B₈ is any nucleic acid, B₇ is A, B₆ is any nucleic acid, B₅ is any nucleic acid, B₄ is C, B₃ is any nucleic acid, B₂ is any nucleic acid and B₁ is C; and ii) a suppressor transfer ribonucleic acid (stRNA). In one embodiment, the nucleic acid antisense oligomer includes, but is not limited to, SEQ ID NOs: 120-184. In one embodiment, the stRNA is aminoacylated. In one embodiment, the stRNA is ser-tRNA^{UGA}. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F) 2′-locked (IN), a 2′-O-methyl (Ome), a 2′-methoxyethyl (Moe) or a mismatched nucleotide. In one embodiment, the nucleic acid antisense oligomer is single stranded. In one embodiment, the nucleic acid antisense oligomer is double stranded.

In one embodiment, the present invention contemplates a nucleic acid antisense oligomer comprising, or consisting of, a plurality of nucleic acids having the sequence of:

5′-C₂₀-C₁₉-C₁₈-C₁₇-C₁₆-C₁₅-C₁₄-C₁₃-C₁₂-C₁₁-C₁₀-C₉₋
C₈₋C₇₋C₆₋C₅₋C₄₋C₃₋C₂₋C₁₋3′

wherein, C₂₀ is any nucleic acid, C₁₉ is any nucleic acid, C₁₈ is any nucleic acid, C₁₇, is any nucleic acid, C₁₆ is any nucleic acid, C₁₅ is any nucleic acid, C₁₄ is any nucleic acid, C₁₃ is any nucleic acid, C₁₂ is any nucleic acid, C₁₁ is any nucleic acid, C₁₀ is G, C₉ is any nucleic acid, C₈ is any nucleic acid, C₇ is A, C₆ is any nucleic acid, C₅ is any nucleic acid, C₄ is C, C₃ is any nucleic acid, C₂ is any nucleic acid and C₁ is C. In one embodiment, the nucleic acid antisense oligomer includes, but is not limited to SEQ ID NOs: 242-298. In one embodiment, the nucleic acid antisense oligomer comprises at least one nucleic acid comprising a chemical modification. In one embodiment, the chemical modification includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the nucleic acid antisense oligomer is single stranded. In one embodiment, the nucleic acid antisense oligomer is double stranded.

In one embodiment, the present invention contemplates a composition comprising: i) a nucleic acid antisense oligomer comprising, or consisting of, a plurality of nucleic acids having the sequence of:

5′-C₂₀-C₁₉-C₁₈-C₁₇-C₁₆-C₁₅-C₁₄-C₁₃-C₁₂-C₁₁-C₁₀-C₉₋
C₈₋C₇₋C₆₋C₅₋C₄₋C₃₋C₂₋C₁₋3′

wherein, C₂₀ is any nucleic acid, C₁₉ is any nucleic acid, C₁₈ is any nucleic acid, C₁₇ is any nucleic acid, C₁₆ is any nucleic acid, C₁₅ is any nucleic acid, C₁₄ is any nucleic acid, C₁₃ is any nucleic acid, C₁₂ is any nucleic acid, C₁₁ is any nucleic acid, C₁₀ is G, C₉ is any nucleic acid, C₈ is any nucleic acid, C₇ is A, C₆ is any nucleic acid, C₅ is any nucleic acid, C₄ is C, C₃ is any nucleic acid, C₂ is any nucleic acid and C₁ is C; and ii) a suppressor transfer ribonucleic acid (stRNA). In one embodiment, the nucleic acid antisense oligomer includes, but is not limited to, SEQ ID NOs: 242-298. In one embodiment, the stRNA is aminoacylated. In one embodiment, the stRNA is ser-tRNA^{UGA}. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the nucleic acid antisense oligomer is single stranded. In one embodiment, the nucleic acid antisense oligomer is double stranded.

In one embodiment, the present invention contemplates a nucleic acid antisense oligomer comprising, or consisting of, a plurality of nucleic acids having the sequence of:

5′-D₂₀-D₁₉-D₁₈-D₁₇-D₁₆-D₁₅-D₁₄-D₁₃-D₁₂-D₁₁-D₁₀-D₉-D₈-
D₇-D₆-D₅-D₄-D₃-D₂-D₁-3′

wherein, D₂₀ is any nucleic acid, D₁₉ is any nucleic acid, D₁₈ is any nucleic acid, D₁₇ is any nucleic acid, D₁₆ is any nucleic acid, D₁₅ is any nucleic acid, D₁₄ is any nucleic acid, D₃ is any nucleic acid, D₁₂ is any nucleic acid, D₁₁, is any nucleic acid, D₁₀ is any nucleic acid, D₉ is any nucleic acid, D₈ is any nucleic acid, D₇ is A, D₆ is any nucleic acid, D₅ is any nucleic acid, D₄ is C, D₃ is any nucleic acid, D₂ is any nucleic acid and D₁ is C. In one embodiment, the nucleic acid antisense oligomer includes, but is not limited to SEQ ID NOs: 496-692. In one embodiment, the nucleic acid antisense oligomer comprises at least one nucleic acid comprising a chemical modification. In one embodiment, the chemical modification includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the nucleic acid antisense oligomer is single stranded. In one embodiment, the nucleic acid antisense oligomer is double stranded.

In one embodiment, the present invention contemplates a composition comprising: i) a nucleic acid antisense oligomer comprising, or consisting of, a plurality of nucleic acids having the sequence of:

5′-D₂₀-D₁₉-D₁₈-D₁₇-D₁₆-D₁₅-D₁₄-D₁₃-D₁₂-D₁₁-D₁₀-D₉₋
D₈₋D₇₋D₆₋D₅₋D₄₋D₃₋D₂₋D₁₋3′

wherein, D₂₀ is any nucleic acid, D₁₉ is any nucleic acid, D₁₈ is any nucleic acid, D₁₇ is any nucleic acid, D₁₆ is any nucleic acid, D₁₅ is any nucleic acid, D₁₄ is any nucleic acid, D₁₃ is any nucleic acid, D₁₂ is any nucleic acid, D₁₁ is any nucleic acid, D₁₀ is any nucleic acid, D₉ is any nucleic acid, D₈ is any nucleic acid, D₇ is A, D₆ is any nucleic acid, D₅ is any nucleic acid, D₄ is C, D₃ is any nucleic acid, D₂ is any nucleic acid and D₁ is C; and ii) a suppressor transfer ribonucleic acid (stRNA). In one embodiment, the nucleic acid antisense oligomer includes, but is not limited to, SEQ ID NO: s 496-692. In one embodiment, the stRNA is aminoacylated. In one embodiment, the stRNA is ser-tRNA^{UGA}. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the nucleic acid antisense oligomer is single stranded. In one embodiment, the nucleic acid antisense oligomer is double stranded.

In one embodiment, the present invention contemplates a method, comprising: a) providing; i) a patient comprising a messenger ribonucleic acid (mRNA) having a nonsense stop codon, wherein the patient exhibits at least one symptom of cystic fibrosis; and ii) a pharmaceutically acceptable composition comprising: A) a nucleic acid antisense oligomer having the sequence of SEQ ID NOs: 1-7, 243, 255, 501 and 528; and B) a suppressor tRNA molecule that is complementary to the nonsense stop codon; and b) administering the pharmaceutically acceptable composition to the patient such that said at least one symptom of cystic fibrosis is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a cystic fibrosis transmembrane conductance regulator protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the cystic fibrosis is caused by a truncated cystic fibrosis transmembrane conductance regulator protein. In one embodiment, the administering further comprises expression of a full length cystic fibrosis transmembrane conductance regulator protein.

In one embodiment, the present invention contemplates a method, comprising: a) providing; i) a patient comprising a messenger ribonucleic acid (mRNA) having a nonsense stop codon, wherein the patient exhibits at least one symptom of Rett Syndrome; and ii) a pharmaceutically acceptable composition comprising: A) a nucleic acid antisense oligomer having the sequence of SEQ ID NOs: 8-18; and B) a suppressor tRNA molecule that is complementary to the nonsense stop codon; and b) administering the pharmaceutically acceptable composition to the patient such that said at least one symptom of Rett Syndrome is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a methyl CpG binding protein 2 protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the Rett Syndrome is caused by a truncated methyl CpG binding protein 2 protein. In one embodiment, the administering further comprises expression of a full length methyl CpG binding protein 2 protein.

In one embodiment, the present invention contemplates a method, comprising: a) providing; i) a patient comprising a messenger ribonucleic acid (mRNA) molecule having a nonsense stop codon, wherein the patient exhibits at least one symptom of chorioderemia; and ii) a pharmaceutically acceptable composition comprising: A) a nucleic acid antisense oligomer having the sequence of SEQ ID NOs: 15-16 and 499, and B) a suppressor tRNA molecule that is complementary to the nonsense stop codon; and b) administering the pharmaceutically acceptable composition to the patient such that said at least one symptom of choroideremia is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a Rab escort protein (REP1). In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN),a 2′-O-methyl (Ome), a 2′-methoxyethyl (Moe) or a mismatched nucleotide. In one embodiment, the choroideremia is caused by a truncated Rab escort protein. In one embodiment, the administering further comprises expression of a full length Rab escort protein.

In one embodiment, the present invention contemplates a method, comprising: a) providing; i) a patient comprising a messenger ribonucleic acid (mRNA) having a nonsense stop codon, wherein the patient exhibits at least one symptom of Duchenne muscular dystrophy; and ii) a pharmaceutically acceptable composition comprising: A) a nucleic acid antisense oligomer having the sequence of SEQ ID NOs: 170, 247, 496, 498, 511, 527, 555, 556, 658, 720 and 721; and B) a suppressor tRNA molecule that is complementary to the nonsense stop codon; and b) administering the pharmaceutically acceptable composition to the patient such that said at least one symptom of Duchenne muscular dystrophy is reduced. In one embodiment, the stRNA is aminoacylated. In one embodiment, the stRNA is ser-tRNA^{UGA} In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a dystrophin protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the Duchenne muscular dystrophy is caused by a truncated dystrophin protein. In one embodiment, the administering further comprises expression of a full length dystrophin protein.

In one embodiment, the present invention contemplates a method, comprising: a) providing; i) a patient comprising a messenger ribonucleic acid (mRNA) having a nonsense stop codon, wherein the patient exhibits at least one symptom of nonspherocytic hemolytic anemia; and ii) a pharmaceutically acceptable composition comprising: A) a nucleic acid antisense oligomer having the sequence of SEQ ID NOs: 497 and 724; and B) a suppressor tRNA molecule that is complementary to the nonsense stop codon; and b) administering the pharmaceutically acceptable composition to the patient such that said at least one symptom of nonspherocytic hemolytic anemia is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a pyruvate kinase protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the nonspherocytic hemolytic anemia is caused by a truncated pyruvate kinase protein. In one embodiment, the administering further comprises expression of a full length pyruvate kinase protein.

In one embodiment, the present invention contemplates a method, comprising: a) providing; i) a patient comprising a messenger ribonucleic acid (mRNA) molecule having a nonsense stop codon, wherein the patient exhibits at least one symptom of an inherited retinal disease; and ii) a pharmaceutically acceptable composition comprising: A) a nucleic acid antisense oligomer having the sequence of SEQ ID NO: 520; and B) a suppressor tRNA molecule that is complementary to the nonsense stop codon; and b) administering the pharmaceutically acceptable composition to the patient such that said at least one symptom of the inherited retinal disease is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a peripherin 2 protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the inherited retinal disease is caused by a truncated peripherin 2 protein. In one embodiment, the administering further comprises expression of a full length peripherin 2 protein.

In one embodiment, the present invention contemplates a method, comprising: a) providing; i) a patient comprising a messenger ribonucleic acid (mRNA) having a nonsense stop codon, wherein the patient exhibits at least one symptom of ataxia-telangiectasia; and ii) a pharmaceutically acceptable composition comprising: A) a nucleic acid antisense oligomer having the sequence of SEQ ID NO: 678; and B) a suppressor tRNA molecule that is complementary to the nonsense stop codon; and b) administering the pharmaceutically acceptable composition to the patient such that said at least one symptom of ataxia-telangiectasia is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a serine/threonine kinase protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN),a 2′-O-methyl (Ome), a 2′-methoxyethyl (Moe) or a mismatched nucleotide. In one embodiment, the ataxia-telangiectasia is caused by a truncated serine/threonine kinase protein. In one embodiment, the administering further comprises expression of a full length serine/threonine kinase protein.

In one embodiment, the present invention contemplates a method, comprising: a) providing; i) a patient comprising a messenger ribonucleic acid (mRNA) having a nonsense stop codon, wherein the patient exhibits at least one symptom of a disorder or disease selected from the group consisting of Miyoshi myopathy, limb-girdle muscular dystrophy type 2B, and distal anterior compartment myopathy; and ii) a pharmaceutically acceptable composition comprising: A) a nucleic acid antisense oligomer having the sequence of SEQ ID NOs: 290 and 655; and B) a suppressor tRNA molecule that is complementary to the nonsense stop codon; and b) administering the pharmaceutically acceptable composition to the patient such that said at least one symptom of the disorder or disease selected from the group consisting of Miyoshi myopathy, limb-girdle muscular dystrophy type 2B, and distal anterior compartment myopathy is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a dysferlin protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the Miyoshi myopathy, limb-girdle muscular dystrophy type 2B or distal anterior compartment myopathy is caused by a truncated dysferlin protein. In one embodiment, the administering further comprises expression of a full length dysferlin protein.

In one embodiment, the present invention contemplates a method, comprising: a) providing; i) a patient comprising a messenger ribonucleic acid (mRNA) molecule having a nonsense stop codon, wherein the patient exhibits at least one symptom of breast or ovarian cancer; and ii) a pharmaceutically acceptable composition comprising: A) a nucleic acid antisense oligomer having the sequence of SEQ ID NOs: 256, 505, 529, 530, 713, 714, 715 and 717; and B) a suppressor tRNA molecule that is complementary to the nonsense stop codon; and b) administering the pharmaceutically acceptable composition to the patient such that said at least one symptom of retinitis pigmentosa is reduced. In one embodiment, the stRNA is aminoacylated. In one embodiment, the stRNA is ser-tRNA^{UGA} In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a breast cancer 1 protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the breast or ovarian cancer is caused by a truncated breast cancer 1 protein. In one embodiment, the administering further comprises expression of a full length breast cancer 1 protein.

In one embodiment, the present invention contemplates a method, comprising: a) providing; i) a patient comprising a messenger ribonucleic acid (mRNA) molecule having a nonsense stop codon, wherein the patient exhibits at least one symptom of Bull's-eye maculopathy; and ii) a pharmaceutically acceptable composition comprising: A) a nucleic acid antisense oligomer having the sequence of SEQ ID NOs: 184, 692 and 712; and B) a suppressor tRNA molecule that is complementary to the nonsense stop codon; and b) administering the pharmaceutically acceptable composition to the patient such that said at least one symptom of Bull's-eye maculopathy is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a ATP binding cassette subfamily A member 4 protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the retinitis pigmentosa is caused by a truncated ATP binding cassette subfamily A member 4 protein. In one embodiment, the administering further comprises expression of a full length ATP binding cassette subfamily A member 4 protein.

In one embodiment, the present invention contemplates a method, comprising: a) providing; i) a patient comprising a messenger ribonucleic acid (mRNA) having a nonsense stop codon, wherein the patient exhibits at least one symptom of familial breast or pancreatic cancer; and ii) a pharmaceutically acceptable composition comprising: A) a nucleic acid antisense oligomer having the sequence of SEQ ID NOs: 152 and 609; and B) a suppressor tRNA molecule that is complementary to the nonsense stop codon; and b) administering the pharmaceutically acceptable composition to the patient such that said at least one symptom of breast or pancreatic cancer is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a partner and localizer of BRACA2 protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN),a 2′-O-methyl (Ome), a 2′-methoxyethyl (Moe) or a mismatched nucleotide. In one embodiment, the familial breast or pancreatic cancer is caused by a truncated partner and localizer of BRACA2 protein. In one embodiment, the administering further comprises expression of a full length partner and localizer of BRACA2 protein.

In one embodiment, the present invention contemplates a method, comprising: a) providing; i) a patient comprising a messenger ribonucleic acid (mRNA) having a nonsense stop codon, wherein the patient exhibits at least one symptom of neurofibromatosis; and ii) a pharmaceutically acceptable composition comprising: A) a nucleic acid antisense oligomer having the sequence of SEQ ID NOs: 521 and 726; and B) a suppressor tRNA molecule that is complementary to the nonsense stop codon; and b) administering the pharmaceutically acceptable composition to the patient such that said at least one symptom of neurofibromatosis is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a neurofibromin protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the neurofibromatosis is caused by a truncated neurofibromin protein. In one embodiment, the administering further comprises expression of a full length neurofibromin protein.

In one embodiment, the present invention contemplates a method, comprising: a) providing; i) a patient comprising a messenger ribonucleic acid (mRNA) molecule having a nonsense stop codon, wherein the patient exhibits at least one symptom of Usher Syndrome; and ii) a pharmaceutically acceptable composition comprising: A) a nucleic acid antisense oligomer having the sequence of SEQ ID NOs: 246 and 504; and B) a suppressor tRNA molecule that is complementary to the nonsense stop codon; and b) administering the pharmaceutically acceptable composition to the patient such that said at least one symptom of Usher Syndrome is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a myosin VIIA protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the Usher syndrome is caused by a truncated myosin VIIA protein. In one embodiment, the administering further comprises expression of a full length myosin VIIA protein.

In one embodiment, the present invention contemplates a method, comprising: a) providing; i) a patient comprising a messenger ribonucleic acid (mRNA) having a nonsense stop codon, wherein the patient exhibits at least one symptom of LAMA2-related muscular dystrophy; and ii) a pharmaceutically acceptable composition comprising: A) a nucleic acid antisense oligomer having the sequence of SEQ ID NOs: 242, 500 and 510; and B) a suppressor tRNA molecule that is complementary to the nonsense stop codon; and b) administering the pharmaceutically acceptable composition to the patient such that said at least one symptom of LAMA2-related muscular dystrophy is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a laminin 2 alpha-2 subunit protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the LAMA2-related muscular dystrophy is caused by a truncated laminin 2 alpha-2 subunit protein. In one embodiment, the administering further comprises expression of a full length laminin 2 alpha-2 subunit protein.

In one embodiment, the present invention contemplates a method, comprising: a) providing; i) a patient comprising a messenger ribonucleic acid (mRNA) having a nonsense stop codon, wherein the patient exhibits at least one symptom of recessive retinitis pigmentosa; and ii) a pharmaceutically acceptable composition comprising: A) a nucleic acid antisense oligomer having the sequence of SEQ ID NOs: 716, 264 and 553; and B) a suppressor tRNA molecule that is complementary to the nonsense stop codon; and b) administering the pharmaceutically acceptable composition to the patient such that said at least one symptom of recessive retinitis pigmentosa is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes an eyes shut homolog protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the recessive retinitis pigmentosa is caused by a truncated eyes shut homolog protein. In one embodiment, the administering further comprises expression of a full length eyes shut homolog.

In one embodiment, the present invention contemplates a method, comprising: a) providing; i) a patient comprising a messenger ribonucleic acid (mRNA) having a nonsense stop codon, wherein the patient exhibits at least one symptom of tuberous sclerosis complex 1; and ii) a pharmaceutically acceptable composition comprising: A) a nucleic acid antisense oligomer having the sequence of SEQ ID NOs: 722, 513, 518 and 572; and B) a suppressor tRNA molecule that is complementary to the nonsense stop codon; and b) administering the pharmaceutically acceptable composition to the patient such that said at least one symptom of tuberous sclerosis complex 1 is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a harmartin protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the recessive retinitis pigmentosa is caused by a truncated hamartin protein. In one embodiment, the administering further comprises expression of a full length hamartin protein.

In one embodiment, the present invention contemplates a method, comprising: a) providing; i) a patient comprising a messenger ribonucleic acid (mRNA) having a nonsense stop codon, wherein the patient exhibits at least one symptom of recessive juvenile retinitis pigmentosa; and ii) a pharmaceutically acceptable composition comprising: A) a nucleic acid antisense oligomer having the sequence of SEQ ID NO: 719; and B) a suppressor tRNA molecule that is complementary to the nonsense stop codon; and b) administering the pharmaceutically acceptable composition to the patient such that said at least one symptom of recessive juvenile retinitis pigmentosa is reduced. In one embodiment, the stRNA is aminoacylated. In one embodiment, the stRNA is ser-tRNA^{UGA} In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a tubby-like protein 1 protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the recessive juvenile retinitis pigmentosa is caused by a truncated tubby-like protein 1 protein. In one embodiment, the administering further comprises expression of a full length tubby-like protein 1 protein.

In one embodiment, the present invention contemplates a method, comprising: a) providing; i) a patient comprising a messenger ribonucleic acid (mRNA) having a nonsense stop codon, wherein the patient exhibits at least one symptom of tuberous sclerosis complex 2; and ii) a pharmaceutically acceptable composition comprising: A) a nucleic acid antisense oligomer having the sequence of SEQ ID NOs: 722, 513, 518 and 572; and B) a suppressor tRNA molecule that is complementary to the nonsense stop codon; and b) administering the pharmaceutically acceptable composition to the patient such that said at least one symptom of tuberous sclerosis complex 2 is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a tuberin protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe) or a mismatched nucleotide. In one embodiment, the tuberous sclerosis complex is caused by a truncated tuberin protein. In one embodiment, the administering further comprises expression of a full length tuberin protein.

In one embodiment, the present invention contemplates a method, comprising: a) providing; i) a patient comprising a messenger ribonucleic acid (mRNA) having a nonsense stop codon, wherein the patient exhibits at least one symptom of progressive pseudorheumatoid dysplasia; and ii) a pharmaceutically acceptable composition comprising: A) a nucleic acid antisense oligomer having the sequence of SEQ ID NOs: 725 and 566; and B) a suppressor tRNA molecule that is complementary to the nonsense stop codon; and b) administering the pharmaceutically acceptable composition to the patient such that said at least one symptom of progressive pseudorheumatoid dysplasia is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a Wingless/Integrated 1 protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN),a 2′-O-methyl (Ome), a 2′-methoxyethyl (Moe) or a mismatched nucleotide. In one embodiment, the progressive pseudorheumatoid dysplasia is caused by a truncated Wingless/Integrated 1 protein. In one embodiment, the administering further comprises expression of a full length Wingless/Integrated 1 protein.

In one embodiment, the present invention contemplates a method, comprising: a) providing; i) a patient comprising a messenger ribonucleic acid (mRNA) having a nonsense stop codon, wherein the patient exhibits at least one symptom of epilepsy; and ii) a pharmaceutically acceptable composition comprising: A) a nucleic acid antisense oligomer having the sequence of SEQ ID NO: 728; and B) a suppressor tRNA molecule that is complementary to the nonsense stop codon; and b) administering the pharmaceutically acceptable composition to the patient such that said at least one symptom of epilepsy is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a progranulin protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN),a 2′-O-methyl (Ome), a 2′-methoxyethyl (Moe) or a mismatched nucleotide. In one embodiment, the epilepsy is caused by a truncated progranulin protein. In one embodiment, the administering further comprises expression of a full length progranulin protein.

In one embodiment, the present invention contemplates a method, comprising: a) providing; i) a patient comprising a messenger ribonucleic acid (mRNA) having a nonsense stop codon, wherein the patient exhibits at least one symptom of hemophilia; and ii) a pharmaceutically acceptable composition comprising: A) a nucleic acid antisense oligomer having the sequence of SEQ ID NOs: 730, 50, 168, 287, 650 and 651; and B) a suppressor tRNA molecule that is complementary to the nonsense stop codon; and b) administering the pharmaceutically acceptable composition to the patient such that said at least one symptom of hemophilia is reduced. In one embodiment, the stRNA is aminoacylated. In one embodiment, the stRNA is ser-tRNA^{UGA}. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a coagulation factor VIII protein. In one embodiment, the nucleic acid antisense oligomer comprises at least one modified nucleotide. In one embodiment, the modified nucleotide includes, but is not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN),a 2′-O-methyl (Ome), a 2′-methoxyethyl (Moe) or a mismatched nucleotide. In one embodiment, the hemophilia is caused by a truncated coagulation factor VIII protein. In one embodiment, the administering further comprises expression of a full length coagulation factor VIII protein.

In one embodiment, the present invention contemplates a nucleic acid antisense oligomer that is at least partially complementary to a messenger ribonucleic acid (mRNA) sequence starting between a +4-+9 nucleotide position downstream of the first nucleotide of a nonsense stop codon. In one embodiment, the nonsense stop codon comprises UAA. In one embodiment, the nonsense stop codon comprises UAG. In one embodiment the nonsense stop codon comprises UGA. In one embodiment, the nonsense stop codon is UGAC. In one embodiment, the nonsense stop codon is UGAG. In one embodiment, the nonsense stop codon is UGAA. In one embodiment, the nonsense stop codon is UGAU. In one embodiment, the nucleic acid antisense oligomer has a sequence selected from the group consisting of SEQ ID NOs: 1-16. In one embodiment, the nucleic acid antisense oligomer comprises at least one nucleotide with a modification. In one embodiment, these modifications include, but are not limited to, a 2′-fluoride (F) modification, a 2′-O-methyl (Ome) modification and a phosphothioate (PS) linkage modification. In one embodiment, the modified nucleic acid antisense oligomer is TCCACTCAGTGTGATTCCAC^{F}. In one embodiment, the modified nucleic acid oligomer is U^{F}CCAC^{F}TCAG^{F}TGTG^{F}ATTC^{F}CAC^{F}.

In one embodiment, the present invention contemplates a composition comprising: i) a nucleic acid antisense oligomer that is at least partially complementary to a messenger ribonucleic acid (mRNA) sequence starting between a +4-+9 nucleotide position downstream of the first nucleotide of a nonsense stop codon. In one embodiment, the composition further comprises a suppressor transfer ribonucleic acid (stRNA) that is complementary to said nonsense stop codon. In one embodiment, the stRNA is aminoacylated. In one embodiment, the stRNA is ser-tRNA^{UGA}. In one embodiment, the nonsense stop codon comprises UAA. In one embodiment, the nonsense stop codon comprises UAG. In one embodiment the nonsense stop codon comprises UGA. In one embodiment, the nonsense stop codon is UGAC. In one embodiment, the nonsense stop codon is UGAG. In one embodiment, the nonsense stop codon is UGAA. In one embodiment, the nonsense stop codon is UGAU. In one embodiment, the nucleic acid antisense oligomer has a sequence selected from the group consisting of SEQ ID NOs: 1-16. In one embodiment, the nucleic acid antisense oligomer comprises at least one nucleotide with a modification. In one embodiment, these modifications include, but are not limited to, a 2′-fluoride (F) modification, a 2′-O-methyl (Ome) modification and a phosphothioate (PS) linkage modification. In one embodiment, the modified nucleic acid antisense oligomer is TCCACTCAGTGTGATTCCAC^{F}. In one embodiment, the modified nucleic acid oligomer is U^{F}CCAC^{F}TCAG^{F}TGTG^{F}ATTC^{F}CAC^{F}.

In one embodiment, the present invention contemplates a nucleic acid antisense oligomer that is at least partially complementary to a messenger ribonucleic acid (mRNA) sequence starting between a +4 and a +9 nucleotide position downstream of the first nucleotide of a nonsense stop codon, crosslinked to a suppressor tRNA.

In one embodiment, the present invention contemplates a nucleic acid antisense oligomer that is at least partially complementary to a messenger ribonucleic acid (mRNA) sequence starting between a +4 and a +9 nucleotide position downstream of the first nucleotide of a nonsense stop codon, crosslinked to readthrough-inducing aminoglycoside.

In one embodiment, the present invention contemplates a nucleic acid antisense oligomer that is at least partially complementary to a messenger ribonucleic acid (mRNA) sequence starting between a +4 and a +9 nucleotide position downstream of the first nucleotide of a nonsense stop codon, crosslinked to another nucleic acid oligomer that is at least partially complementary to the same mRNA.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The generic term “+#(##)” as used herein, refers to an antisense nucleotide binding nomenclature format. For example, the antisense oligonucleotide designation of “+8(70)” means that the “+8” refers to the registered binding nucleotide which is eight (8) nucleotides downstream of the first nucleotide of a nonsense/premature stop codon and the “(70)” is the calculated (predicted) melting temperature for an analogous DNA-DNA duplex having the same sequence of the antisense oligo. The other antisense oligo designations presented herein follow the same format and interpretation.

As used herein, the term “antisense”, “nucleic acid antisense oligomer” or “R-ASO” is used in reference to nucleic acid sequences (e.g., DNA, RNA or DNA-RNA) which are complementary to at least a region of a specific RNA sequence (e.g., mRNA). In particular, such antisense oligomers may have complete or partial complementarity to an mRNA beginning +4 to +9 nucleotides downstream of a nonsense (premature) codon. Antisense oligomers may be produced by any method, including but not limited to, phosphoramidite chemical synthesis or synthesis by splicing the gene(s) of interest in a reverse orientation to a viral promoter which permits the synthesis of a coding strand. Once introduced into a cell, this oligonucleotide strand combines with the mutant mRNA produced by the cell to form duplexes. These duplexes then promote translation of the full-length mRNA. In this manner, cell phenotypes may be generated which result in the alleviation of symptoms of a genetic disease.

The term “aminoglycoside” as used herein, refers to any organic molecule that contains amino sugar substructures. Clinically, an aminoglycoside is a medicinal and bacteriologic category of traditional gram-negative antibacterial medications that inhibit protein synthesis and contain as a portion of the molecule an amino-modified glycoside. For example, an aminoglycoside includes, but is not limited to, G418, gentamicin, amikacin, tobramycin, kanamycin, streptomycin and neomycin. It is generally known that the administration of conventional concentrations (i.e., doses) of an aminoglycoside results in side effects in a large percentage of patients. Such side effects include those systems related to, but are not limited to, auditory, renal and vestibular.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” as used herein, refers to an art-accepted causal relationship between a genetic mutation and a medical condition or disease. For example, it is art-accepted that a patient having a gene comprising a mutation that generates a nonsense stop codon has, or is a risk for, a genetic disease or disorder or disease.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA or their modified analogs of genomic or synthetic origin which may be single- or double-stranded, and represent the sense or antisense strand.

The term “modified nucleic acid”, as used herein, refers to any nucleic acid molecule having modified backbone, sugar, nucleobase, or novel base or base pair. Such modifications may include, but are not limited to, a phosphate (P) linkage, a phosphothioate (PS) linkage, a methylphosphonate, a phosphorodiamidate morpholino (PMO), a 2′-H (dN), a 2′-hydroxy (rN), a 2′-fluoride (F), 2′-locked (IN),a 2′-O-methyl (Ome), a 2′-methoxyethyl (Moe) or a mismatched nucleotide.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue. When used in reference to an amino acid sequence refers to fragments of that amino acid sequence. The fragment may range in size from 2 amino acid residues to the entire amino acid sequence minus one amino acid residue.

The term “nucleic acid sequence” and “nucleotide sequence” as used herein, refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and refer to a sequence of amino acids.

The term “portion” when used in reference to an amino acid sequence refers to fragments of that amino acid sequence. The fragment may range in size from 2 amino acid residues to the entire amino acid sequence minus one amino acid residue.

The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “functionally equivalent codon”, as used herein, refers to different codons that encode the same amino acid. This phenomenon is often referred to as “degeneracy” of the genetic code. For example, six different codons encode the amino acid arginine.

A “variant” of a protein is defined as an amino acid sequence which differs by one or more amino acids from a polypeptide sequence or any homolog of the polypeptide sequence. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs including, but not limited to, DNAStar® software.

A “variant” of a nucleotide is defined as a novel nucleotide sequence which differs from a reference oligonucleotide by having deletions, insertions and substitutions. These may be detected using a variety of methods (e.g., sequencing, hybridization assays etc.).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues.

A “substitution” results from the replacement of one or more nucleotides or amino acids by different nucleotides or amino acids, respectively.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is at least partially complementary, i.e., “substantially homologous,” to a nucleic acid sequence is one that at least at least partially inhibits a completely complementary sequence from hybridizing to a target nucleic acid sequence. A nucleotide sequence which is fully complementary, e.g., “completely homologous”, to a nucleic acid sequence is on that completely inhibits a completely complementary sequence from hybridizing to a target nucleic acid sequence, The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

As used herein, the term “hybridization”, “hybridized” or “hybridizing” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

The term “nucleic acid antisense oligomers” are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “nucleic acid molecule encoding”, “RNA sequence encoding”, “DNA sequence encoding,” and “DNA encoding” refer to the order or sequence of (deoxy)ribonucleotides along a strand of (deoxy)ribonucleic acid. The order of these (deoxy)ribonucleotides determines the order of amino acids along the polypeptide (protein) chain. The DNA and RNA sequences thus code for an amino acid sequence.

As used herein, the term “coding region” or “open reading frame (ORF)” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

### BRIEF DESCRIPTION OF THE FIGURES

FIG. 1 presents a clarification of different nucleotide register nomenclatures between that used to describe the present invention and that disclosed in Kar et al., “Induction of Translational Readthrough across the Thalassemia-Causing Premature Stop Codon in β-Globin-Encoding mRNA” Biochemistry 59(1):80-84 (2020; online Oct. 2, 2019).

FIG. 2A-C presents an exemplary illustration of translation of a hypothetical protein from an mRNA molecule with either a nonsense (premature) stop codon or a natural (wild type) stop codon.

FIG. 2A: A truncated protein is produced at a premature stop codon (left). A full length protein is produced at a natural stop codon (right).

FIG. 2B: An illustration of termination factors which recognizes and binds to either a nonsense stop codon (left) or a natural stop codon (right). The termination factor sterically “pulls” the stop codon into ribosome, thereby releasing the translated protein.

FIG. 2C: An illustration of a site-specific nucleic acid antisense oligomer positioned downstream of a premature stop codon that interferes with a steric “pull” or other interaction with the termination factor on the ribosome, thereby allowing a readthrough of the complete mRNA and translation of a full length protein.

FIG. 2D: A truncated protein is produced at a premature stop codon.

FIG. 2E: A full length protein is produced at a natural stop codon upon stop ASO-induced stop codon readthrough.

FIG. 3A-B illustrates a rabbit reticulocyte luciferase mRNA translation assay.

FIG. 3A: Step-wise illustration showing the translation of a luciferase mRNA molecule with a stop codon into full length luciferase protein that emits light (arrows).

FIG. 3B: Exemplary data of light intensity (e.g., relative luminescence units (RLUs)) during luciferase mRNA translation. Left panel: The light intensity pattern as a function of time (e.g., seconds). Middle panel: The rate of luciferase translation shown by light intensity fluctuation over time. Right panel: Depicts the maximal achievable rate of the luciferase translation shown by light intensity fluctuation over time.

FIG. 3C: A scheme for calculating the translation efficiency from time-progress luminescence curves. RLU-relative light units.

FIG. 4 illustrates one embodiment of a CAN1 arginine permease gene/luciferase expression construct (Can1-luc). Upper panel: A schematic of the construct showing the relative position of a nonsense stop codon and a Can1 open reading frame and a luciferase gene with a natural terminal stop codon. Lower panel: An mRNA of the Can1-luc construct with a TGA premature stop codon and a nucleic acid antisense oligomer hybridized to the Can1-luc construct at the +8 nucleotide position downstream of the nonsense stop codon.

FIG. 5 presents exemplary data showing readthrough of a CAN1-luc expression construct nonsense stop codon with a nucleic acid antisense oligomer hybridized at the +8 nucleotide position downstream of the nonsense stop codon.

FIG. 6 presents one embodiment of a cystic fibrosis gene/nanoluciferase expression construct (511-565 CFTR). Upper panel: A schematic of the construct showing the relative position of a nonsense stop codon (UGAG or UGAC) within a cystic fibrosis open reading frame (CFTR) and a luciferase gene with a natural terminal stop codon. Lower panel: A CFTR mRNA construct with a TGA premature stop codon and a nucleic acid antisense oligomer hybridized to the CFTR mRNA construct at the +8 nucleotide position downstream of the nonsense stop codon.

FIG. 7 presents exemplary data showing a readthrough analysis of a CFTR nonsense stop codon (UGAC) construct expression in the presence of a nucleic acid antisense oligomer targeted to various nucleotide positions downstream of the nonsense stop codon as well as having different melting temperatures (Tₘs).

FIG. 8 presents exemplary data showing the effect of the aminoglycoside G418 on 511-565 CFTR mRNA construct expression. Increasing concentrations of G418 decreases the expression of wild type construct without nonsense stop codon (GGAG) while increasing expression of nonsense stop codon constructs (UGAG, UGAC).

FIG. 9 presents exemplary data showing the synergistic effect of a +8(66) nucleic acid antisense oligomer and the aminoglycoside G418 on CFTR mRNA construct expression readthrough of premature stop codons. Also shown is a +8 nucleic acid antisense oligomer in combination with G418 that decreases the effective concentration for an aminoglycoside.

FIG. 10 presents exemplary data showing a synergistic effect of a +8(47) nucleic acid antisense oligomer and the aminoglycoside G418 on CFTR mRNA construct expression readthrough of premature stop codons.

FIG. 11 presents exemplary data correlating nanoluciferase activity with expressed protein level to validate readthrough promotion by the nucleic acid antisense oligomers as contemplated herein.

FIG. 12A-B provides a schematic of a luciferase-based assay (TermiLuc, Susorov, 2020) to identify the loss of a translation termination step to promote readthrough.

FIG. 12A: A schematic of the Termi-Luc assay.

FIG. 12B: One example of a eukaryotic termination complex.

FIG. 13 presents exemplary data showing that +7 and +8 nucleic acid antisense oligomers inhibit translation termination in a sequence specific-manner in the Termi-Luc assay.

FIG. 14 presents an illustrative structure of a modified nucleic acid antisense oligomer with a 2′-fluoride substitution.

FIG. 15 presents exemplary data showing readthrough promotion with fluoride-modified nucleic acid antisense oligomers using a rabbit reticulocyte lysate (RRL) assay.

FIG. 16 presents exemplary data showing promotion of CFTR nonsense codon readthrough by a combination of G418 and a modified nucleic acid antisense oligomer in a dose-dependent manner. O=modified oligomer; G=G418 in the culture of cell expressing CFTR with premature stop codon G542X, fused with HRP to measure chemiluminescence resulting from full-length protein expression.

FIG. 17A-B presents one embodiment of a full length Mecp2 gene/nanoluciferase expression construct.

FIG. 17A: A schematic of the construct showing the relative position of a nonsense stop codon within a full length Mecp2 open reading frame and a luciferase gene with a natural terminal stop codon.

FIG. 17B: Exemplary data showing the effect of a +8 nucleic acid antisense oligomer on readthrough efficiency for four (4) nonsense stop codons responsible for Rett syndrome.

FIG. 18A-D present exemplary data showing next-generation bulk sequencing results of PCR amplicons using TOPO cloning (e.g., allelic exclusion) method.

FIG. 19A-D present exemplary data showing an analysis of gene expression using both quantitative polymerase chain reaction and Western Blot.

FIG. 20A-C present exemplary data of a TECC/24 conductance assay performed at one (1) week post-filter seeding.

FIG. 21A-B present exemplary data of transepithelial resistance of the CFF-16HIBEge cell lines.

FIG. 22 presents one embodiment of a CFTR mRNA with a nonsense stop codon bound to a suppressor tRNA and a +8 nucleic acid antisense oligomer.

FIG. 23 illustrates a chemiluminescent assay showing light emission when nonsense codon readthrough is promoted by a suppressor tRNA.

FIG. 24A-B presents exemplary data showing synergistic readthrough of cystic fibrosis nonsense stop codons (G542X) with a combination of a +8 nucleic acid antisense oligomer plus a suppressor ser-tRNA^{UGA}.

FIG. 24A: Data comparison at the UGAC nonsense stop codon.

FIG. 24B: Data comparison at the UGAG nonsense stop codon.

FIG. 25 presents an exemplary raw data plot of that presented in FIG. 24.

FIG. 26 presents exemplary data showing synergistic readthrough of a UGAU nonsense stop codon in a chorioderemia mRNA with a combination of a +8 nucleic acid antisense oligomer plus a suppressor ser-tRNA^{UGA}.

FIG. 27 presents exemplary data showing synergistic readthrough of a UGAG nonsense stop codon in a Rett syndrome mRNA with a combination of a +8 nucleic acid antisense oligomer plus a suppressor ser-tRNA^{UGA}.

FIG. 28 presents exemplary data showing successful readthrough of a CFTR-HRP construct with a G542X (cystic-fibrosis causing mutation) stop codon in FTR cells (20,000 per well).

FIG. 29 presents an experimental system to measure translation efficiency of readthrough using a nanoluciferase reporter as a full-protein readout in rabbit reticulocyte lysate.

FIG. 29A: The scheme for model mRNAs used for in vitro translation reactions.

FIG. 29B: The scheme of in vitro translation experiment.

FIG. 30 presents one possible scheme for calculating the translation efficiency from time-progress luminescence curves. Partial data from FIG. 31C is used as an example. RLU-relative light units.

FIG. 31 presents exemplary data showing that nucleic acid antisense oligomers (R-ASOs) induce sequence-specific readthrough of nonsense codons. RLU-relative light units; WB-western blotting; CDS-coding sequence; black arrow marks mRNA degradation product observed with R-ASOs. Control=mRNA before translation in RRL

FIG. 31A: Example of a model mRNA with a nonsense-stop-codon-containing fragment of the CFTR gene (G542X mutant) followed by nanoluciferase.

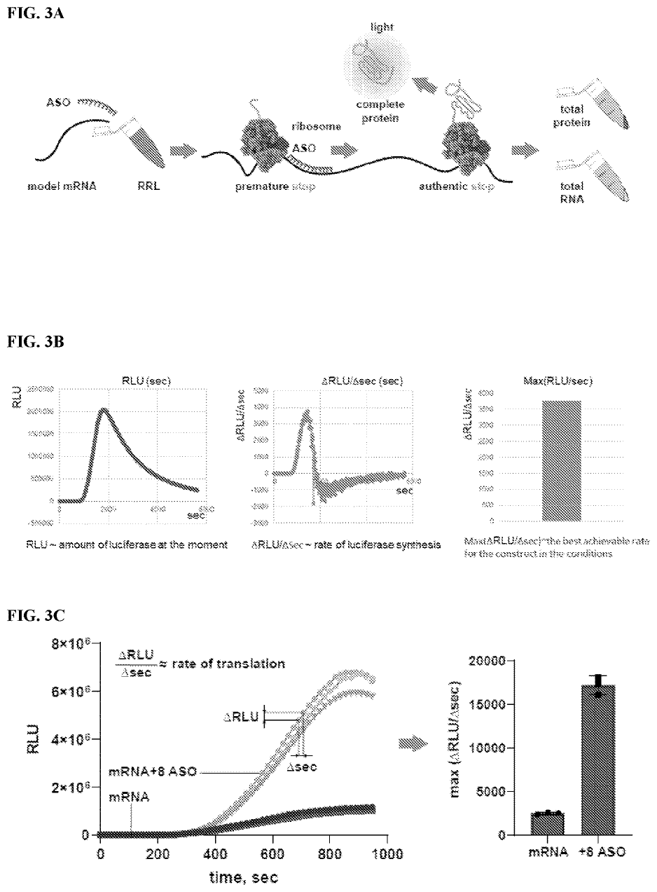

FIG. 3IB: Translation efficiencies of model mRNAs with nonsense stop codons +1UGAC+4 and +1UGAG+4 are substantially lower than a model mRNA without nonsense stop codons (i.e. sense GGA codon); (middle panel) western blotting of full-length protein product (via an anti N-terminus antibody); (lower panel) RNA gel showing mRNA levels at the end of translation reaction (agarose electrophoresis; AE with fluorescent probes complimentary to CDS).

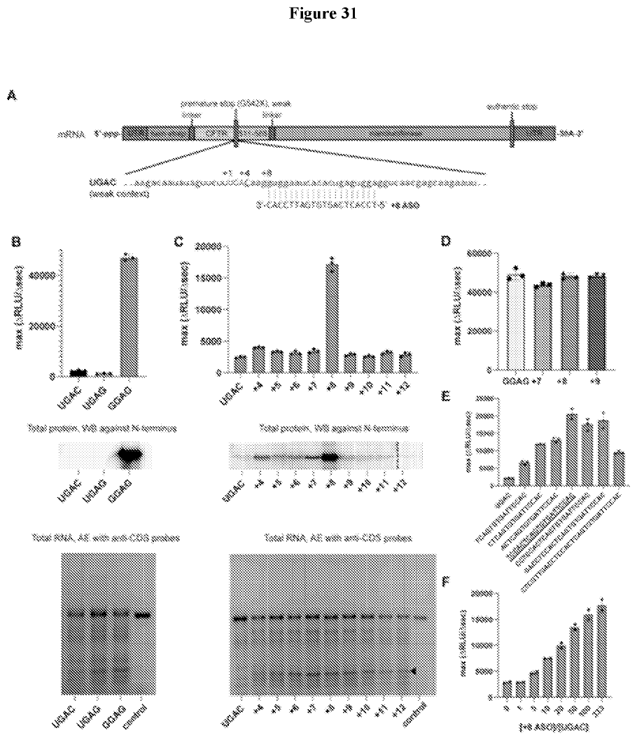

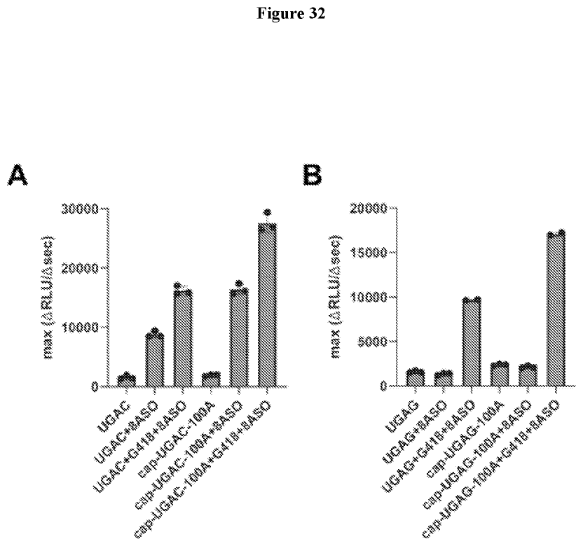

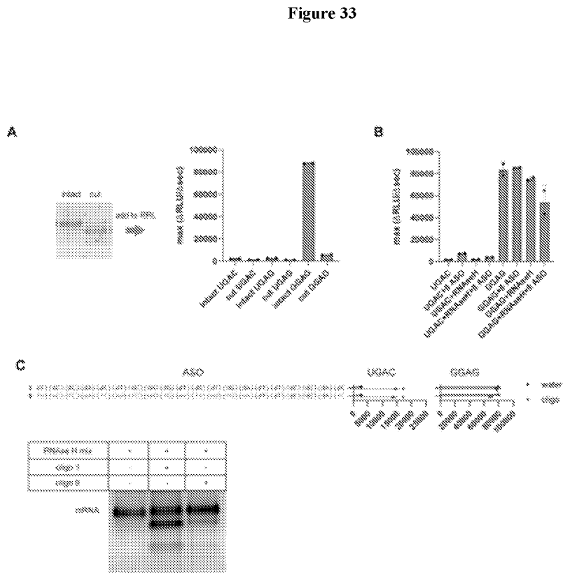

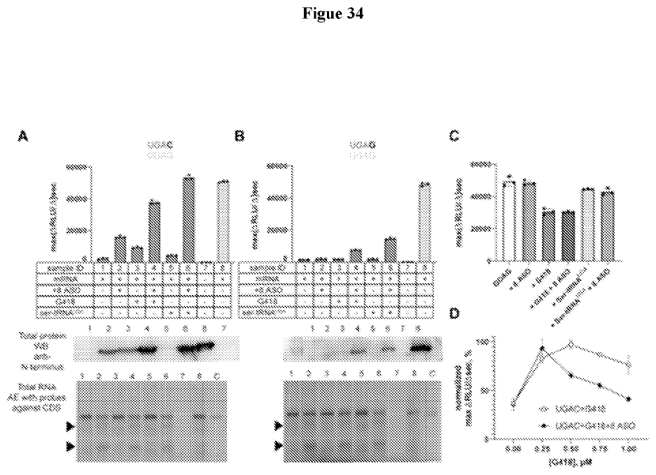

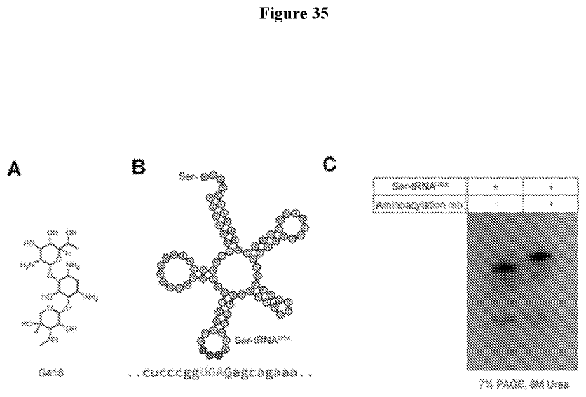

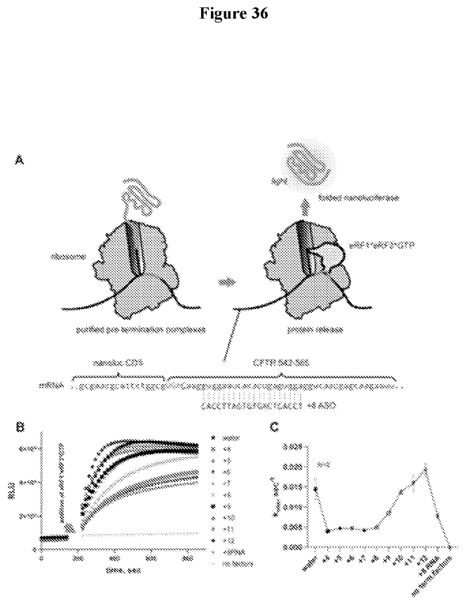

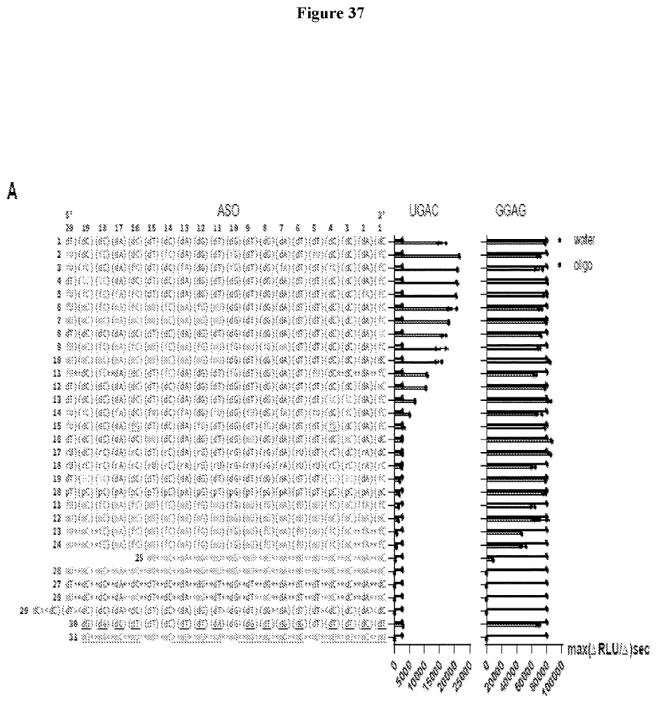

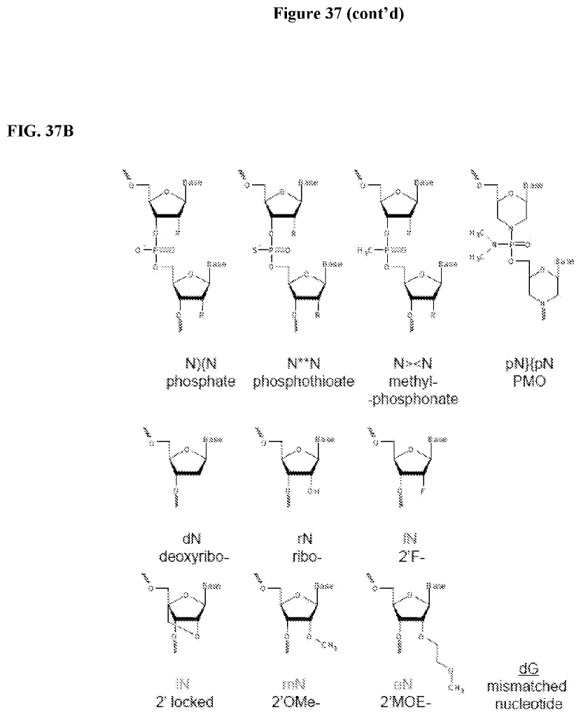

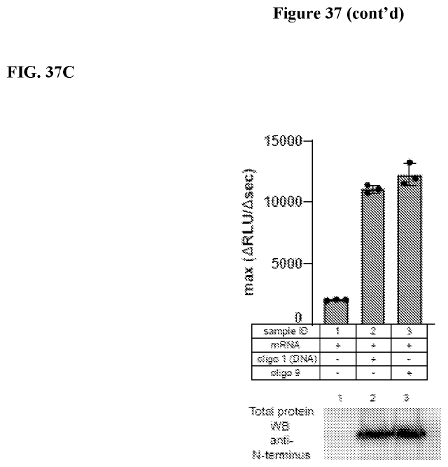

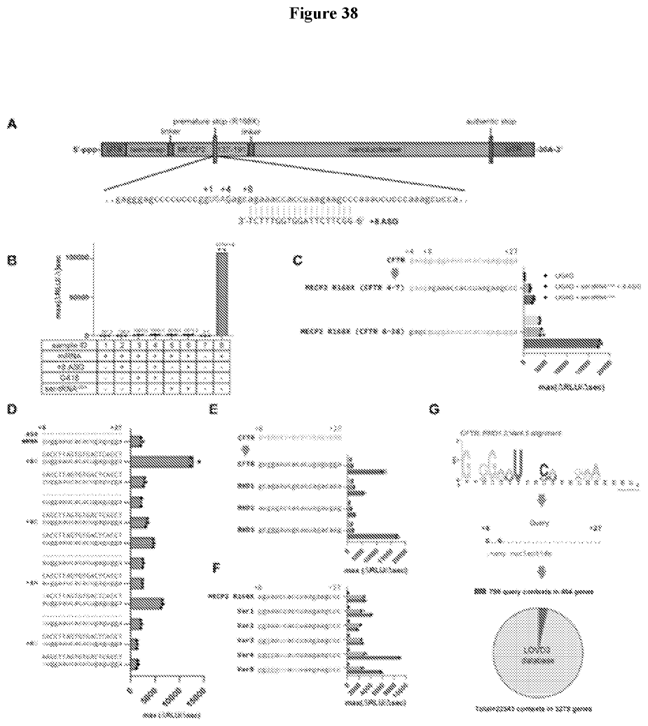

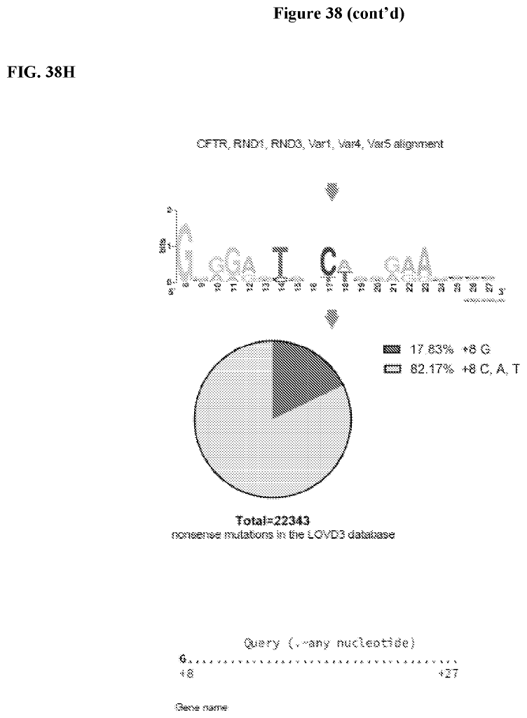

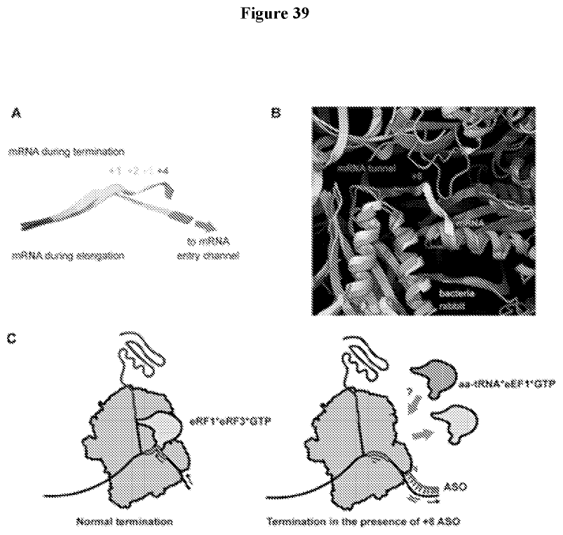

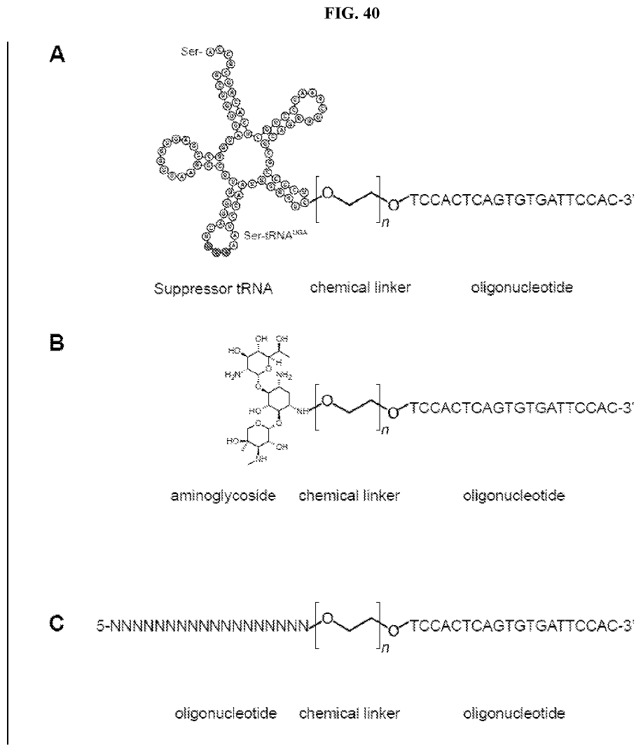

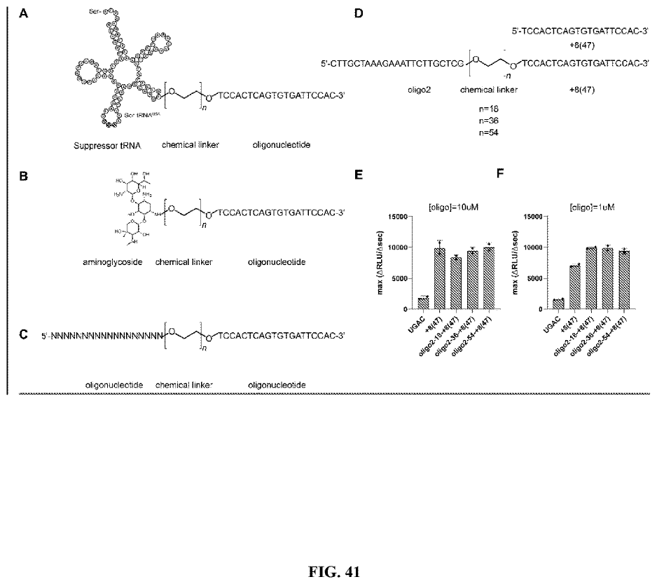

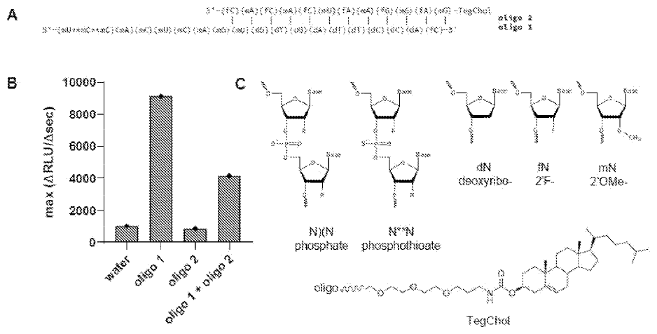

FIG. 31C: Translation efficiencies of nonsense stop codon-containing mRNA with nucleic acid antisense oligomers annealing to different positions downstream of the +1UGAC+4 nonsense codon resulting full-length protein and mRNA levels.

FIG. 31D: R-ASOs do not affect translation efficiency of wild-type (nonsense-free) mRNA.

FIG. 31E: The length of R-ASO affects the efficiency of nonsense readthrough. A 20-mer+8 R-ASO is used in most experiments unless otherwise stated (underlined).

FIG. 31F: Dependence of readthrough efficiency on a +8 R-ASO (20-nt) concentration.

FIG. 32 presents a comparison of readthrough efficiencies in RRL for model mRNAs.

FIG. 32A: UGAC before and after capping and poly(A)-tail elongation.

FIG. 32B: UGAG before and after capping and poly(A)-tail elongation.

FIG. 33 presents exemplary data showing that RNAse H cleavage is not responsible for translation readthrough.

FIG. 33A: Translation efficiency of intact CFTR mRNAs and mRNA treated with RNAse H and a +8 nucleic acid antisense oligomer (R-ASO).

FIG. 33B: Translation efficiency of intact CFTR mRNAs with and without RNAse H added to RRL externally.

FIG. 33C: Chemically modified R-ASO demonstrate reduced in vitro RNAse H activity. RLU-relative luminescence units.

FIG. 34 presents exemplary data showing enhancement of nucleic acid antisense oligomer (R-ASO)-induced readthrough by G418 and suppressor tRNA. Arrows mark additional mRNA degradation products observed with R-ASOs; C-control mRNA before translation in RRL; RLU-relative light units; CDS-coding sequence.

FIG. 34A: Combinations of +8 R-ASO with G418 or tRNASer(UGA) induce efficient readthrough of UGAC stop codons; (middle panel) western blotting of full-length protein product (via an anti N-terminus antibody); (lower panel) Agarose electrophoresis RNA gel showing mRNA levels at the end of translation reactions (with fluorescent probes against CDS). C-control for mRNA before translation.

FIG. 34B: Combinations of +8 R-ASO with G418 or tRNASer(UGA) induce efficient readthrough of UGAG stop codons; (middle panel) western blotting of full-length protein product (via an anti N-terminus antibody); (lower panel) Agarose electrophoresis RNA gel showing mRNA levels at the end of translation reactions (with fluorescent probes against CDS). C-control for mRNA before translation.

FIG. 34C: Effect of readthrough inducers on translation of wild-type (nonsense-free) mRNA sequence.

FIG. 34D: Effect of G418 concentrations, with and without +8 R-ASO, on the readthrough of the UGAC mRNA.

FIG. 35 illustrates representative nonsense stop codon readthrough promoting agents that synergize with nucleic acid antisense oligomers (R-ASOs).

FIG. 35A: A representation of the chemical structure of the aminoglycoside G418.

FIG. 35B: A cloverleaf model of a suppressor ser-tRNA^{UGA}.

FIG. 35C: A photomicrograph of a denaturing PAGE analysis of ser-tRNA^{UGA} before and after in vitro aminoacylation.

FIG. 36 presents exemplary data showing the dependence of translation termination on the downstream annealing position of the nucleic acid antisense oligomer (R-ASO) relative to a nonsense stop codon.

FIG. 36A: Schematic of the Termi-Luc assay to measure the kinetics of nanoluciferase release from purified pre-termination complexes.

FIG. 36B: Nanoluciferase luminescence upon release is inhibited by some R-ASOs.

FIG. 36C: Dependence of the release rates on the position of the R-ASO downstream the stop codon.

FIG. 37 presents exemplary data that nucleotides having chemical modifications affect readthrough efficiency of R-ASOs. C-control mRNA before translation in RRL; RLU-relative luminescence units; CDS-coding sequence.

FIG. 37A: Readthrough efficiency (UGAC) and translation efficiency (GGAG) are differentially affected by nucleotide/backbone modifications of a CFTR +8 R-ASO.

FIG. 37B: Chemical structures of modifications tested.

FIG. 37C: Comparison of translation efficiency, full-length protein and mRNA stability for unmodified and modified (#9) R-ASOs on the UGAC CFTR mRNA. Arrows mark additional mRNA degradation products observed with R-ASOs.

FIG. 38 presents exemplary data showing that positional annealing of a nucleic acid antisense oligomer (R-ASO) on an mRNA having a nonsense stop codon affects readthrough efficiency.

FIG. 38A: Schematic of an mRNA construct with the fragment of Mecp2 containing a Rett-syndrome-causing R168X mutation.

FIG. 38B: Translation efficiency of the nonsense stop codon-containing and wild-type stop codon (nonsense-free GGAG) Mecp2 mRNA in the presence of readthrough inducers.

FIG. 38C: Readthrough efficiencies of UGAG Mecp2 mRNA constructs carrying local substitutions with CFTR mRNA sequences.

FIG. 38D: Readthrough efficiencies of UGAC CFTR mRNA constructs carrying +8 position nucleic acid substitutions.

FIG. 38E Readthrough efficiencies of three UGAG CFTR-like sequences randomized between the +8-+27 positions.

FIG. 38F: Readthrough efficiencies of UGAG Mecp2 mRNAs with substitutions in the +8-+27 region.

FIG. 38G: A first exemplary consensus sequence analysis between the +8-+27 positions of human mRNAs with disease-causing nonsense stop codons determined by a LOVD3 database query.

FIG. 38H: A second exemplary consensus sequence analysis between the +8-+27 positions of human mRNAs with disease-causing nonsense stop codons determined by a LOVD3 database query.

FIG. 39 presents an illustrative model for nucleic acid antisense oligomer (R-ASO) induced readthrough of nonsense stop-codons.

FIG. 39A: Comparison of mRNA positions in the ribosomal A site during elongation and termination. PDB codes: 5LZS and 5LZT, respectively.

FIG. 39B: Comparison of mRNA entry channels of bacterial (yellow) and mammalian (orange) ribosomes. PDB codes: 6BY1 (ribosome B) and 5LZS, respectively. The structures were superimposed by ribosomal proteins underlining the entry channel (S3, S4, S5).

FIG. 39C: Proposed mechanism for nonsense readthrough induced by R-ASOs binding downstream of the stop codon.

FIG. 40 presents exemplary examples and readthrough data for nucleic acid antisense oligomer conjugates:

FIG. 40A: A conjugate between an nucleic acid antisense oligomer and a suppressor tRNA;

FIG. 40B: A conjugate between an nucleic acid antisense oligomer and an aminoglycoside;

FIG. 40C: A conjugate between an nucleic acid antisense oligomer and another oligonucleotide.

FIG. 40D: A conjugate between two nucleic acid antisense oligomers (e.g., a +8(47) oligo and oligo2) with linkers of different lengths (e.g., 18-mer; 36-mer; 54-mer);

FIG. 40E: Exemplary data showing readthrough induced by a +8(47) nucleic acid antisense oligomer or by the linker conjugates shown in FIG. 40D annealing to CFTR sequence downstream to +8(47), at 10 μM;

FIG. 40F: Exemplary data showing readthrough induced by a +8(47) nucleic acid antisense oligomer or by the linker conjugates shown in FIG. 40D annealing to CFTR sequence downstream to +8(47), at 1 μM. The data show that the conjugates have higher efficiency at lower concentrations.

FIG. 41 presents representative duplex complexes of nucleic acid antisense oligomers that promote readthrough.

FIG. 41A: A hybridization scheme between two R-ASOs.

FIG. 41B: An effect of the separate oligonucleotides as well as of their complementary complex on the expression of the model mRNA having UGAC stop codon in RRL.

FIG. 41C: Representative chemical modifications for R-ASOs.

### DETAILED DESCRIPTION OF THE INVENTION

This invention is related to the field of genetic engineering. In particular, it is related to compositions and methods to treat genetically-based diseases and disorder or diseases that are caused by the translation of non-functional proteins from an mRNA with a nonsense (premature) stop codon. Nonsense stop codon readthrough results in a full-length protein and restores protein function. For example, a combination of a suppressor transfer ribonucleic acid (stRNA) and nucleic acid antisense oligomers are contemplated that promote translation readthrough of mRNA nonsense (premature) stop codons.

### I. Nonsense Stop Codons

Nonsense mutations account for more than 10% of genetic disorder or diseases including, but not limited to, cystic fibrosis, Rett syndrome, and hereditary cancers. A nonsense mutation results in expression of a truncated protein, and therapeutic strategies aim at restoring full-length protein expression. Most strategies under development, including small-molecule aminoglycosides, suppressor tRNAs, or the targeted degradation of termination factors, lack specificity towards the disease-causing nonsense mutation, resulting in off-target translation errors. The data presented herein demonstrate an mRNA-specific strategy for nonsense mutation's stop codon readthrough. For example, nucleic acid antisense oligomers (R-ASOs) induce readthrough of nonsense codons, resulting in high yields of full-length protein.

Nonsense mutations—sense codons mutated to stop codons—cause premature termination of translation, the production of truncated proteins, and nonsense-mediated mRNA decay, resulting in loss- or gain-of-function phenotypes. Restoring full-length translation is therefore the primary goal of treating nonsense-associated diseases. Indeed, even partial restoration of full-length product could provide significant therapeutic benefit.

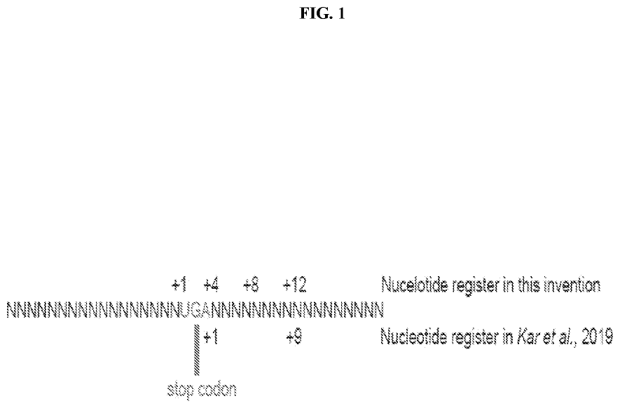

Several strategies have been tested to induce the “read-through” of a stop codon as the mRNA is translated by the ribosome. Whereas sense codons are recognized and decoded by aminoacyl-tRNAs (aa-tRNA), stop codons—UAA, UAG, and UGA—are recognized by release factor eRF1 in eukaryotes. Upon stop-codon recognition, eRF1 catalyzes the irreversible hydrolysis of the peptidyl-tRNA ester linkage, releasing the peptide from the ribosome. For a read-through to occur, the stop codon must be read by an aa-tRNA rather than by eRF1, so that an amino acid is incorporated, allowing translation to continue to the normal stop codon at the end of the open reading frame. See, FIG. 1B. Therapeutic approaches to nonsense readthrough aim to enhance stop-codon decoding by an aa-tRNA or to inhibit termination or both. For example, a suppressor tRNA is a modified tRNA whose anticodon binds to a stop codon and outcompetes eRF1. Ko et al., Mol Ther Nucleic Acids, 28:685-701 (2022). Small-molecule approaches include ribosome-binding compounds, such as aminoglycoside G418, which are thought to both inhibit termination and induce miscoding (Susorov et al. 2020; Wangen and Green 2020; Lawson et al. 2021). Other small-molecule strategies downregulate or inhibit eRF1 (Gurzeler et al. 2023a; Sharma et al. 2021; Carnes et al. 2003), inhibit nonsense-mediated decay of the mRNA (Keeling et al. 2014; Dabrowski et al. 2018; Gurzeler et al. 2023b), or act via less understood or debatable mechanisms (Martins-Dias and Romio 2021). Such approaches lack specificity toward the mRNA of interest and cause miscoding or readthrough of authentic stop codons, yielding aberrant proteins (Lueck et al. 2019; Wang et al. 2022; Wangen and Green 2020).

Structural and biochemical studies demonstrated differences between decoding and termination mechanisms. Whereas aa-tRNA recognizes a codon by forming three base pairs in the decoding center, eRF1 interacts with the stop codon and with the nucleotide immediately after the stop codon, as if “pulling” the mRNA into the entry tunnel (Brown et al. 2015a; Shao et al. 2016; Matheisl et al. 2015a). Indeed, a longer mRNA fragment is protected by the ribosome during termination than during aa-tRNA decoding (Alkalaeva et al. 2006; Ingolia et al. 2011). Thus, we hypothesize that mechanisms interfering with mRNA dynamics at the entry tunnel might inhibit termination and stimulate nonsense readthrough.

The data presented herein show that nucleic acid antisense oligomers (R-ASOs) induce readthrough of nonsense codons and that R-ASOs can induce efficient readthrough of some nonsense stop codon contexts, while other mRNA contexts result in lower readthrough that nevertheless might be therapeutically relevant if achieved in patients. Using a recently developed kinetic assay (Susorov et al. 2020), R-ASOs are shown to inhibit translation termination in a context-specific manner. Individually, or combined with low amounts of other readthrough inducers including, but not limited to, G418 or suppressor tRNAs, R-ASOs synergistically increase readthrough, yielding more product than the sum of the two approaches by themselves.

Nonsense stop codon readthrough efficiency depends on both the sequence context near the nonsense stop codon and the specific binding position of the R-ASO relative to the nonsense stop codon. Moreover, R-ASOs synergize with non-specific readthrough agents, including aminoglycoside G418 or suppressor tRNA, making them effective at lower concentrations, suggesting an advantages clinical approach to reduce the toxicity of these compounds. The data presented herein demonstrate that R-ASOs act to reduce the efficiency of nascent peptide release from a ribosome at the specific nonsense codon. Further, the data identifies R-ASO nucleotide positions comprising ribose ring chemical modifications which do not decrease R-ASO readthrough activity.

### II. Conventional Stop Codon Readthrough Methods

There have been numerous efforts to identify therapeutics that would readthrough a premature stop codon and restore full-length protein translation. Small molecules, such as aminoglycoside antibiotics, result in systematic miscoding of mRNAs and are of limited therapeutic value. Although they also result in stop-codon readthrough with a mutant mRNA, broad miscoding of cellular mRNAs makes such molecules toxic and generally poor therapeutic agents. Dabrowski et al. “Advances in therapeutic use of a drug-stimulated translational readthrough of premature termination codons” Mol Med 24 (2018); and Keeling et al., “Therapeutics Based on Stop Codon Readthrough”. Annu Rev Genomics Hum Genet. (2014).

The main reported adverse effects of aminoglycosides are ototoxicity, nephrotoxicity, and neuromuscular blockade. Avent et al., “Current use of aminoglycosides: indications, pharmacokinetics and monitoring for toxicity” Intern Med J. 41(6):441-449 (2011). Conventional dosing of an aminoglycoside is usually about 3-5 mg/kg/day given intravenously/intramuscularly (IV/IM) divided every 8 hours. An extended dosing interval is about every 24 hours or more at 4-7 mg/kg/dose IV once/day.

Aminoglycoside antibiotics are used as a conventional treatment of pulmonary exacerbations of cystic fibrosis (CF) and slow the decline in lung function which ultimately causes the death of most patients. The prognosis of CF has improved, and thus side effects of treatments have become increasingly important. Prayle et al., “Side effects of aminoglycosides on the kidney, ear and balance in cystic fibrosis” Thorax 65(7):654-658 (2010).

Observational studies suggest that the morbidity from side effects of aminoglycosides is disturbingly common, and that aggressive treatment may lead to more side effects. One common side effect of aminoglycosides is renal toxicity. Studies vary in their definition of toxicity, but approximately 5-10% of (non-CF) adult patients receiving an aminoglycoside have a significant increase in serum creatinine. Meyer R D., “Risk factors and comparisons of clinical nephrotoxicity of aminoglycosides” Am J Med 80:119-125 (1986).

Although toxicity is an aminoglycoside class effect, experimental data suggest that gentamicin is more toxic than tobramycin and amikacin. De Broe et al., “Choice of drug and dosage regimen. Two important risk factors for aminoglycoside nephrotoxicity” Am J Med 80:115-118 (1986). A quantitative overview of randomised controlled trials (RCTs) reached broadly the same conclusions. Buring et al., “Randomized trials of aminoglycoside antibiotics: quantitative overview” Rev Infect Dis 10:951-7 (1988).

Manipulation of an aminoglycoside dosing regimen provides a cost-effective and simple method of reducing kidney injury. Given the saturable uptake of aminoglycosides, it has been reported that a single daily dose would be expected to be less nephrotoxic than the same daily dose in three divided doses. For example, a large randomised trial of tobramycin for patients with CF, established that there is equal efficacy with a single daily dosing regimen as with a multiple daily dosing regimen, a finding confirmed in a subsequent meta-analysis. In a paediatric group receiving a single daily dose, the serum creatinine level decreased during the course of treatment compared with a rise in the group receiving three divided doses. In further support of renal safety, in the once daily arm the rise in NAG was 33% less than in the group receiving a three times daily regimen in both adults and children. Smyth et al., “Once versus three-times daily regimens of tobramycin treatment for pulmonary exacerbations of cystic fibrosis—the TOPIC study: a randomised controlled trial” Lancet 365:573-578 (2008); and Smyth et al., “Once-daily versus multiple-daily dosing with intravenous aminoglycosides for cystic fibrosis” Cochrane Database Syst Rev 2006; (3):CD002009. The pharmacokinetics of aminoglycosides are complicated by a circadian rhythm in elimination. In the once daily group, most of whom received their antibiotics in the evening, there was a lower elimination rate of tobramycin than in the three times daily group. Touw et al., “Population pharmacokinetics of tobramycin administered thrice daily and once daily in children and adults with cystic fibrosis” J Cyst Fibros 6:327-333 (2007) There may be a diurnal variation in renal clearance of the drug, with decreased clearance occurring at night. This would lead to increased exposure of the kidneys to aminoglycoside during the course of the illness if the drug is administered at night compared with the morning.

A selection of numerous studies reports auditory toxicity as measured by Pure Tone Audigrams (PTAs) in CF patients. For example, a study of seventy (70) patients recruited from a CF clinic and, using a definition of ≥2 thresholds of >20 dB or one of >25 dB, they found an overall prevalence of hearing impairment of 17% when given aminoglycosides. Mulheran et al., “Occurrence and risk of cochleotoxicity in cystic fibrosis patients receiving repeated high-dose aminoglycoside therapy” Antimicrob Agents Chemother 45:2502-2509 (2001).

It has been reported that DNA oligonucleotides complementary to a beta-globin mRNA at +1 or +9 nucleotides downstream of an artificially introduced premature stop codon “UAG” can induce translational readthrough in cells. Kar et al., “Induction of Translational Readthrough across the Thalassemia-Causing Premature Stop Codon in β-Globin-Encoding mRNA” Biochemistry 59(1):80-84 (2020; online Oct. 2, 2019). However, these DNA oligonucleotides have not been shown to be an effective therapeutic for any known genetic disorder or disease.

It should be noted that the DNA oligo starting position nomenclature system of Kar et al. differs from that used in the present invention. In particular, Kar's +1 and +9 positions correspond to the +4 and +12 positions (respectively) as presented herein (see FIG. 1). Consequently, DNA oligos that are complementary to an mRNA sequence starting at the +5-+8 nucleotide position downstream of the first nucleotide of a premature stop codon have not been previously reported. Further, as Kar et al. does not disclose any oligonucleotides complementary for the CFTR and Mecp2 genes, the +4-+9 nucleotide positioning of antisense downstream of the first nucleotide of a premature stop codon have not been previously reported. It is noted that the data herein shows that R-ASOs placed further downstream (e.g., positions +9, +12) of the first nucleotide of the nonsense codon did not promote readthrough for CFTR.

### III. Nonsense Codon-Associated Protein Release Factors

In one embodiment, the present invention contemplates compositions and methods to induce efficient mRNA-specific readthrough of nonsense stop (premature) codons resulting in minimal off-target side effects. Although it is not necessary to understand the mechanism of an invention, it is believed that such an approach relies on structural differences between the cellular recognition of stop codons and sense codons.

For example, cellular recognition of stop codons is believed to be mediated by protein release factors (i.e., eRF1 in eukaryotes). Recognition occurs in an A-site codon where an eRF1 protein interacts with an mRNA nucleotide sequence at a stop codon and the following nucleotide (for example, UGAC or UAAA). By contrast, triplet sense codons are recognized by tRNA, where the mRNA sequence downstream of the A-site codon is then threaded through a ribosomal mRNA tunnel and exits into solution. Thus, an eRF1 recognition protein requires that an mRNA be “pulled” into the A-site codon, while tRNA does not.

Oligonucleotides that base-pair with mRNA next to the ribosomal tunnel were tested to determine if they could: i) limit mRNA mobility; ii) make stop-codon recognition by eRF1 inefficient; and iii) make misreading the stop-codon by tRNA efficient, thus resulting in readthrough, iv) or act via a different mechanism to promote readthrough.

### IV. Suppressor Transfer Ribonucleic Acids

Transfer RNA (tRNA) is believed to be an adaptor molecule composed of RNA, typically 76 to 90 nucleotides in length (in eukaryotes). Sharp et al. (1985) “Structure and transcription of eukaryotic tRNA genes” CRC Critical Reviews in Biochemistry 19 (2): 107-144. tRNA has been observed to serve as a physical link between the mRNA and the amino acid sequence of proteins. Transfer RNA (tRNA) does this by carrying an amino acid to the protein synthesizing machinery of a cell called the ribosome. Complementation of a 3-nucleotide codon in a messenger RNA (mRNA) by a 3-nucleotide anticodon of the tRNA results in protein synthesis based on the mRNA code. As such, tRNAs are a necessary component of translation, the biological synthesis of new proteins in accordance with the genetic code.

In eukaryotic cells, tRNAs are transcribed by RNA polymerase III as pre-tRNAs in the nucleus. Govindan et al., (2018) “Development of Assay Systems for Amber Codon Decoding at the Steps of Initiation and Elongation in Mycobacteria”. Journal of Bacteriology 200(22). RNA polymerase III recognizes two highly conserved downstream promoter sequences: the 5′ intragenic control region (5′-ICR, D-control region, or A box), and the 3′-ICR (T-control region or B box) inside tRNA genes. The first promoter begins at +8 of mature tRNAs and the second promoter is located 30-60 nucleotides downstream of the first promoter. The transcription terminates after a stretch of four or more thymidines. Sharp (1982). “The minimum intragenic sequences required for promotion of eukaryotic tRNA gene transcription” Nucleic Acids Research 10(18):5393-5406; and Dieci et al., (2007) “The expanding RNA polymerase III transcriptome” Trends in Genetics 23(12): 614-622.

The structure of tRNA includes, but is not limited to, a primary structure, a secondary structure (usually a cloverleaf structure), and a tertiary structure. Itoh et al., (2013) “Tertiary structure of bacterial serenocysteine tRNA” Nucleic Acids Research 41(13):6729-6738. It is generally believed that tRNA tertiary structure has an L-shaped 3D structure that allows them to fit into the P and A sites of the ribosome. The cloverleaf secondary structure becomes the 3D L-shaped tertiary structure through a coaxial stacking of the helices, which is a common RNA tertiary structure motif. It has been observed that tRNAs generally include the following structures:
i) A 5′-terminal phosphate group;
ii) An acceptor stem that is a 7- to 9-base pairing between a 5′-terminal nucleotide with a 3′-terminal nucleotide which contain a CCA 3′-terminal group that attaches to an amino acid). In general, such 3′-terminal tRNA-like structures are referred to as ‘genomic tags’.
iii) A CCA tail comprising a cytosine-cytosine-adenine sequence at the 3′ end of the tRNA molecule to which an aminoacyl is covalently bonded to a 3′-hydroxyl group.
iv) A D arm is a 4- to 6-bp stem ending in a loop that often contains dihydrouridine.
v) An anticodon arm that is a 5-bp stem whose loop contains a complementary anticodon sequence; and
vi) A T arm that is a 4- to 5-bp stem ending in a loop that may contain a TΨC sequence where Ψ is pseudouridine.

Suppressor tRNAs (stRNAs) can be used to incorporate unnatural amino acids at nonsense codons placed in the coding sequence of a gene. For example, an initiator tRNA (e.g., tRNA^{fMetY} having a CUA anticodon encoded by a metY gene has been used to initiate translation at the amber stop codon UAG. This type of tRNA is called a nonsense suppressor tRNA because it suppresses the translation stop signal that normally occurs at UAG codons. The amber codon initiator tRNA inserts methionine and glutamine at UAG codons preceded by a strong Shine-Dalgarno sequence. The amber initiator tRNA has been shown to be orthogonal to the wild type AUG start codon. Vincent et al., (2019). “Measuring Amber Initiator tRNA Orthogonality in a Genomically Recoded Organism” ACS Synthetic Biology 8(4): 675-685; and Govindan et al., (2018) “Development of Assay Systems for Amber Codon Decoding at the Steps of Initiation and Elongation in Mycobacteria”. Journal of Bacteriology 200(22).

### V. +4-+9 Nucleic Acid Antisense Oligomer Screening

In one embodiment, the present invention contemplates nucleic acid antisense oligomers that bind to mRNA starting at, or between, the +4-+9 nucleotides downstream of the first nucleotide of a nonsense stop codon. The data presented herein show that nucleic acid antisense oligomers that bind mRNA starting at the +4-+9 nucleotide position (i.e., +4, +5, +6, +7, +8, +9 positions) downstream from the first nucleotide of a premature mRNA stop codon (+1 position, see FIG. 1) successfully promoted readthrough. See, Table 1.

TABLE 1
Exemplary +4-+9
Nucleic Acid Antisense Oligomers

| SEQ | | Gene/ |
|---|---|---|
| ID | Nucleotide Sequence | Position |
| 1 | CCTCCACTCAGTGTGATTCCACCTTC | CFTR +4 |
| 2 | CCACTCAGTGTGATTCCACC | CFTR +7 |
| 3 | GACCTCCACTCAGTGTGATTCCACC | CFTR +7 |
| 4 | CTCAGTGTGATTCCAC | CFTR +8 |
| 5 | ACTCAGTGTGATTCCAC | CFTR +8 |
| 6 | TCCACTCAGTGTGATTCCAC | CFTR +8 |
| 7 | CTCGTTGACCTCCACTCAGTGTGATTCCAC | CFTR +8 |
| 8 | CTGAAGCTGACCCTCAGGCC | MECP2 +8 |
| 9 | CGGGGAGTGTGGTGGCAG | MECP2 +8 |
| 10 | TGCAGGAGACCGTACTCCCC | MECP2 +8 |
| 11 | GGCTTCTTAGGTGGTTTCTG | MECP2 +7 |
| 12 | GGCTTCTTAGGTGGTTTCT | MECP2 +8 |
| 13 | GGGCTTCTTAGGTGGTTTC | MECP2 +9 |
| 18 | TCTTTGGTGGATTCTTCGG | MECP2 +8 |
| 14 | CCTGGTAATATTTTTAAACTCTGC | CHM +7 |
| 15 | TCCTGGTAATATTTTTAAACTCTG | CHM +8 |
| 16 | GAATCCTGGTAATATTTTTAAACTCT | CHM +9 |

### A. Structure and Positioning

The data presented herein demonstrate that nucleic acid antisense oligomers annealing to an mRNA sequence downstream of a premature stop codon promotes translation readthrough. Surprisingly, the most effective annealing site for nucleic acid antisense oligomers is the +8 position downstream of the premature stop codon, where the +1 position is the first nucleotide of the premature stop codon. It was observed that not all nucleic acid antisense oligomers positioned downstream of a premature stop codon were equal in promoting readthrough. Readthrough with nucleic acid antisense oligomers that anneal starting at positions +4, +7 and +9 were lower than with those starting at +8, yet were more efficient than background readthrough, rendering the range of antisense oligomers binding between +4 through +9 sensitive to the promotion of translation readthrough of stop codons.

Translation readthrough of a truncated protein around a nonsense stop codon can be different from the readthrough of a full-length protein. For example, stop codons are known to differ in their efficiency of translation termination and subsequent release of a truncated protein. For example, mutations resulting in a UGAC nonsense stop codon are much less efficient (e.g., a weak stop codon) in translation termination than a wild type UAAA stop codon (e.g., a strong stop codon), as a purine (A, G) nucleotide at position +4 renders translation termination more efficient than a pyrimidine (C, U) at position +4. Thus, UGAC—a “weak” stop codon—is more prone to readthrough than the “strong” UAAA stop codon. Indeed, most studies testing small molecules report most efficient readthrough of the UGAC stop codon, while UAAA or UGAG can be completely resistant to readthrough.

The data presented herein demonstrate nucleic acid antisense oligomers that efficiently readthrough and translate a functional protein from mRNAs with “weak” premature stop codons and/or mRNAs with “strong” stop codons either individually or in combination with an aminoglycoside. The data suggest that nucleic acid antisense oligomers complementary to a mRNA nucleotide sequence at the +4-+9 positions downstream of the first nucleotide of a premature stop codon are effective therapeutic candidates for genetic diseases caused by nonsense mutations. These findings show that complementarity of an aberrant mRNA is specific, yet versatile, as nucleic acid sequences (i.e., RNA, LNA and other modifications) can bind to mRNAs having nonsense stop codons that cause various genetic diseases.

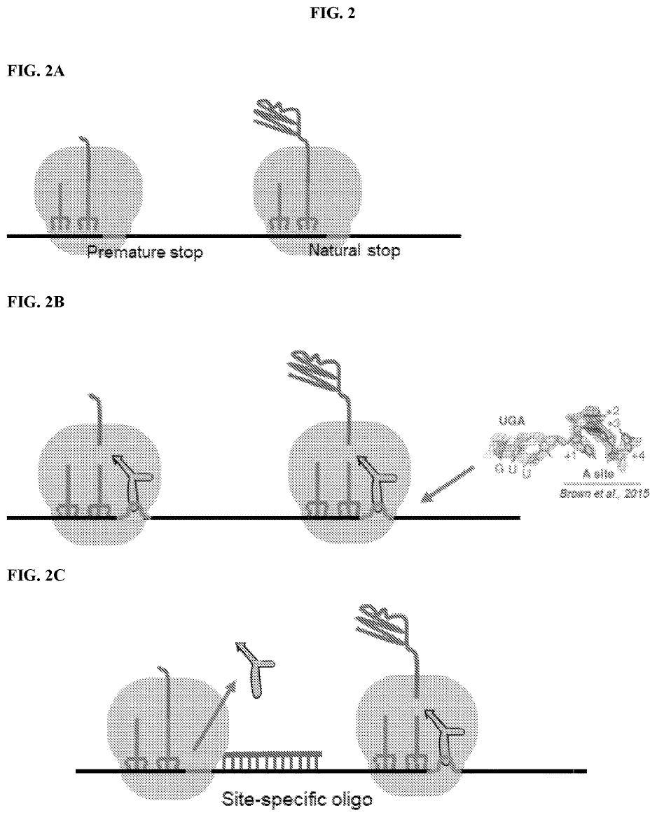

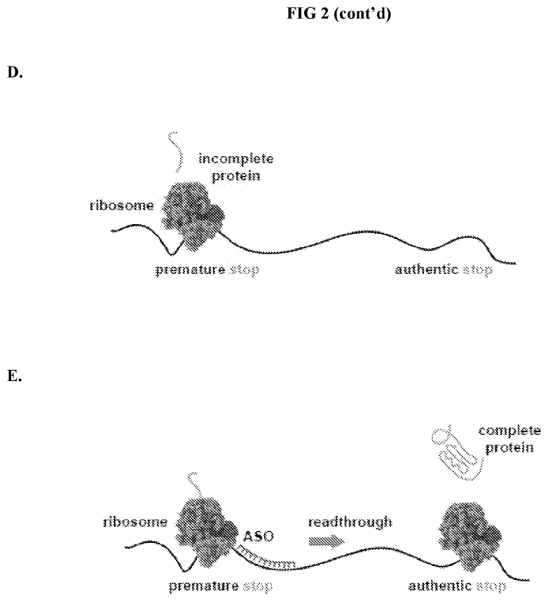

Some genetic diseases are caused by an mRNA molecule having a nonsense stop codon because a truncated protein has been translated and released. In contrast, if an mRNA molecule has a natural (wild type) stop codon in its proper position a full-length protein is translated and released. See, FIG. 2A. Translation termination protein factors function to bind to, and “pull” stop codons into a ribosome resulting in the release of proteins from ribosomes which prevents further mRNA translation. See, FIG. 2B. Site-specific nucleic acid antisense oligomers positioned downstream from nonsense stop codons prevent “pulling” of the stop codon into the ribosome and thus inhibit early protein translation termination leading to readthrough of a premature stop codon and translation of a full-length protein. See, FIG. 2C.

The data presented herein was collected using a translation assay with a commercial cellular extract (i.e., a rabbit reticulocyte lysate). See, FIG. 3A and FIG. 3B. Preliminary data found that nucleic acid antisense oligomers placed at a +8 nucleotide position from an mRNA nonsense stop codon (with +1 position corresponding to the U of the stop codon) resulted in superior readthrough of two different mRNA sequences with nonsense stop codons, while other downstream position placements resulted in less efficient readthrough. Further, the data show that readthrough efficiency is substantially increased by a combination of the nucleic acid antisense oligomer with low concentrations of an aminoglycoside (e.g., G418) thereby providing restoration of 30-40% of functional protein translation.

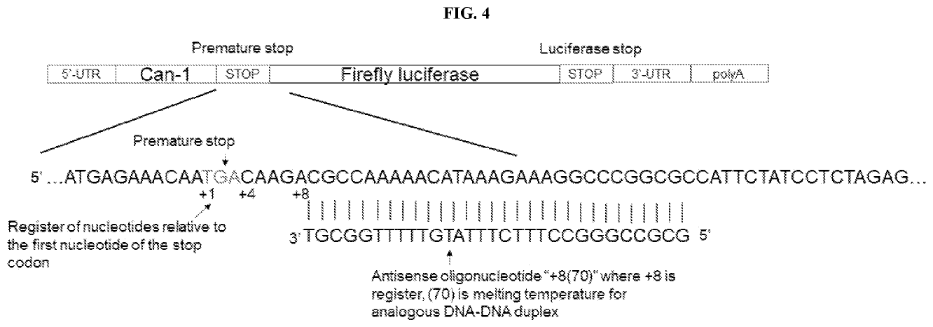

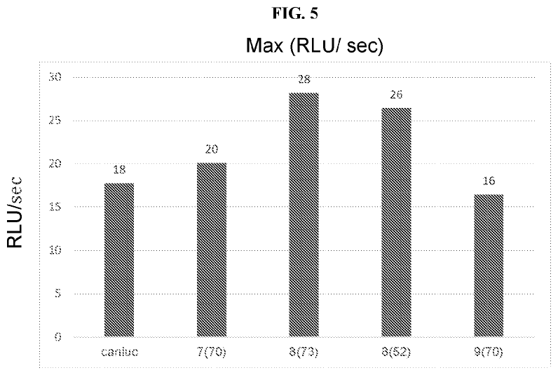

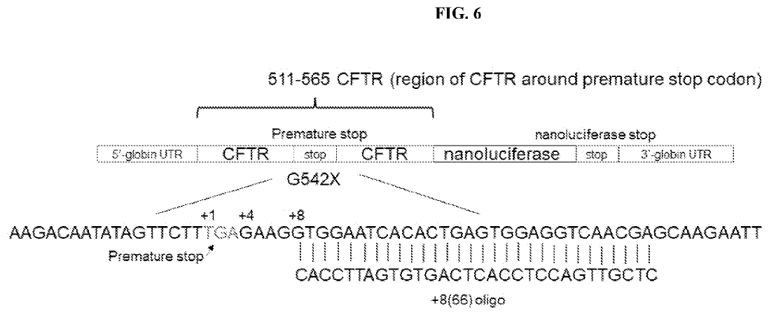

The nucleic acid antisense oligomers contemplated herein were validated using an mRNA encoding a premature stop codon and a luciferase gene. The basic reporter methodology was used a CAN1 mRNA encoding an arginine permease amino acid transporter protein. The CAN1 mRNA positions a TAG premature stop codon at the terminus of the CAN1 open reading frame subsequently followed by a luciferase open reading frame. See, FIG. 4. A +8(70) nucleic acid antisense oligomer having the nucleic acid sequence of 5′-GCGCCGGGCCTTTCTTTATGTTTTTGGCGT-3′ was then positioned at the +8 nucleic acid position downstream of the first nucleotide of the premature stop codon which increased readthrough of a nonsense stop codon in a CAN1-luc mRNA. See, FIG. 5.

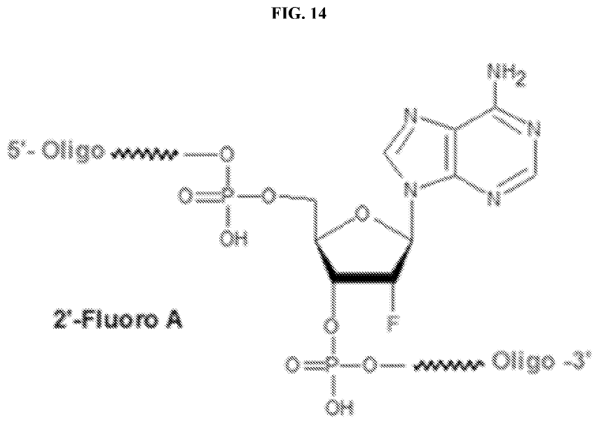

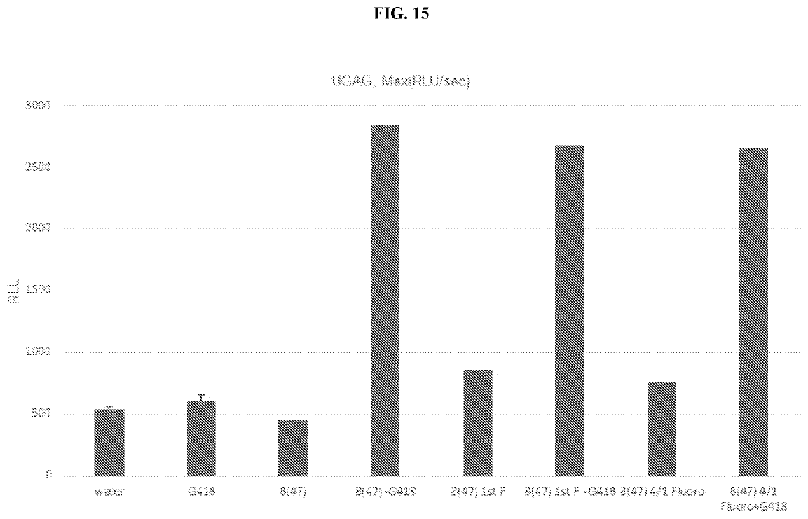

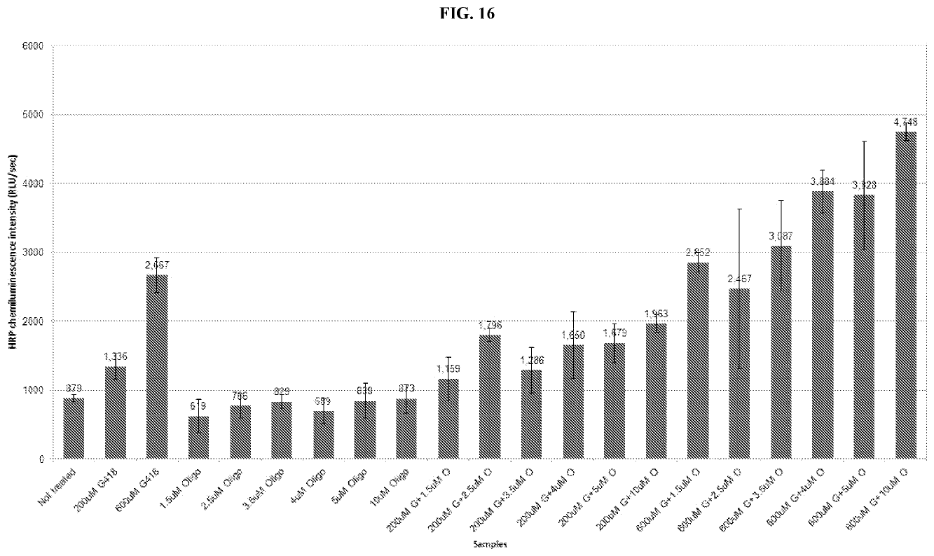

The +4-+9 nucleic acid antisense oligomers as described herein may further comprise at least one nucleotide with a ribose ring modification. These modification include, but are not limited to, a 2′-fluoride (F) modification, a 2′-O-methyl (Ome) modification and a phosphothioate (PS) linkage modification. See, FIG. 14. For example, +8(47) nucleic acid antisense oligomer were modified with a fluoride at least one nucleic acid (NAr): i) one modification: TCCACTCAGTGTGATTCCAC^{F}; and ii) six modifications: U^{F}CCAC^{F}TCAG^{F}TGTG^{F}ATTC^{F}CAC^{F}. The data presented herein demonstrates that these modified nucleic acid antisense oligomers promote readthrough in an RRL assay with improved stability and/or efficiency, and in mammalian cell culture. See, FIG. 15 and FIG. 16.

Fischer rat thyroid (FRT) cells expressing CFTR (G542X) and horseradish peroxidase (HRP) fusion proteins were cultured at 37° C. and 5% CO₂ in a Ham's F-12, Coon's Modification (Sigma-Aldrich, St. Louis, MO, #F6636) buffer with 10% fetal bovine serum (FBS; Thermo Fisher Scientific, #26140-079), 1% penicillin-streptomycin (Thermo Fisher Scientific, #15140-122), and 100 μg/mL hygromycin B (Thermo Fisher Scientific, #10687010). Then, these FRT cells were seeded at a density of 2×10⁴ cells/well in Costar 96 well plate. Twenty-four (24) hours after seeding, FRT cells expressing CFTR (G542X) were incubated for another twenty-four (24) hours with or without different concentrations of G418 and/or the following modified 8(47) antisense oligo: U^{F}CCAC^{F}TCAG^{F}TGTG^{F}ATTC^{F}CAC^{F}.

An HRP luminescence assay was then performed after the cells were washed three times with 1×DPBS, followed by incubation with SuperSignal West Femto HRP substrate (15 μL/well, Thermo Fisher Scientific, #34096) for 5 min at room temperature. The HRP-catalyzed luminescence was read with a Tecan microplate reader under the following conditions: room temperature; no shake/no delay; integration time, 0.1 s; read height, 8.00 mm. The data show that a combination of G418 and the nucleic acid antisense oligomer results in a dose-response related synergy of nonsense codon readthrough. See, FIG. 16.

These data suggest that a chemically-modified nucleic acid antisense oligomer, together with G418, improves the readthrough of premature stop codon in cells expressing a disease-causing variant of CFTR (with G542X mutation).

### B. Suppressor Transfer Ribonucleic Acid Readthrough Enhancement

Preliminary attempts to promote nonsense codon readthrough by delivery of suppressor tRNAs demonstrated decoding stop codons and inserting an amino acid. Wang et al., “AAV-delivered suppressor tRNA overcomes a nonsense mutation in mice” Nature 604(7905):343-348 (2022). But suppressor tRNAs, alone, are not specific to a particular stop codon in a single mRNA. Instead, suppressor tRNAs also result in readthrough of normal stop codons, resulting in elongated and potentially dysfunctional proteins (non-specific readthrough).

In one embodiment, the present invention contemplates a composition comprising a nucleic acid antisense oligomer and a suppressor tRNA. In one embodiment, the nucleic acid antisense oligomer is complementary to an mRNA comprising a nonsense stop codon. In one embodiment, the suppressor tRNA hybridizes to the nonsense stop codon. In one embodiment, the nonsense stop codon causes a disease or medical disorder or disease by releasing a truncated nascent protein from a ribosome.

Although it is not necessary to understand the mechanism of an invention, it is believed that the stRNA/oligomer combination induces an efficient and mRNA-specific readthrough of nonsense stop codons, resulting in minimal off-target side effects. Based upon the structural biology of mRNA translation on the ribosome, the present approach relies on structural differences between cellular recognition of nonsense stop codons and sense stop codons.

For example, recognition of stop codons by proteins called release factors (eRF1 in eukaryotes) occurs at the A site where eRF1 interacts with four (4) nucleotides of mRNA (e.g., the stop codon and the following nucleotide: for example, UGAC or UAAA). Subsequently, the mRNA downstream of the A-site codon is threaded through the ribosomal mRNA tunnel and exits into solution. By contrast, stRNAs recognizes and binds to the three-nucleotide sense codon without any subsequent secondary or tertiary mRNA conformational changes. Thus, eRF1 recognition requires mRNA to be “pulled” into the A site, while stRNA does not.

Nucleic acid antisense oligomers that base-pair with mRNA next to the ribosomal tunnel were tested to determine if they limit mRNA mobility and cause inefficiency of eRF1 stop-codon recognition or improve the efficiency of tRNA stop-codon misreads, thus resulting in readthrough. The data presented herein show that nucleic acid antisense oligomers bind mRNA downstream of a nonsense stop codon and induce readthrough. In one embodiment, the present data has now shown the unexpected and surprising result of a dramatic improvement in the efficiency and specificity of readthrough of an mRNA with a nonsense stop codon when using a combination of nucleic acid antisense oligomer with a suppressor tRNA at a concentration that is ineffective when given alone. This approach substantially improves nonsense stop codon readthrough promotion efficacy of both the nucleic acid antisense and suppressor tRNA. Although it is not necessary to understand the mechanism of an invention, it is believed that this approach can alleviate non-specific stop codon readthrough by suppressor tRNA.

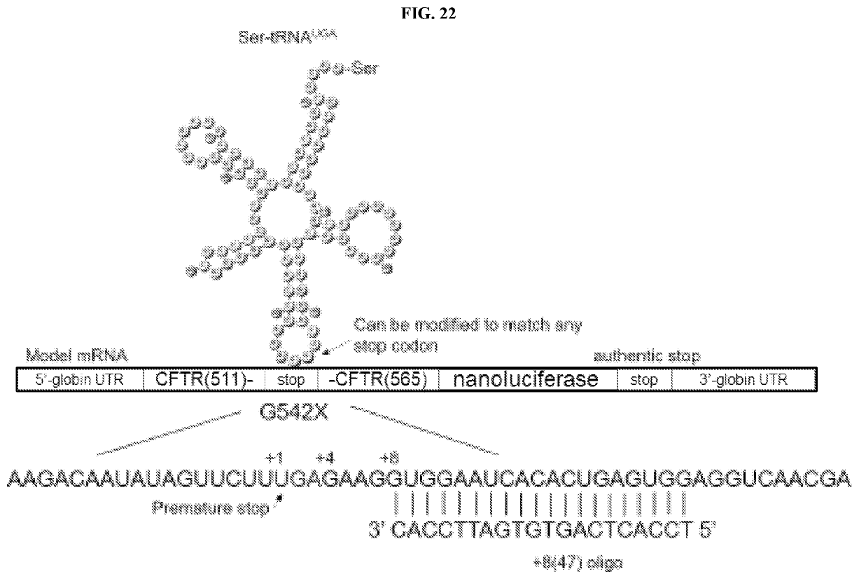

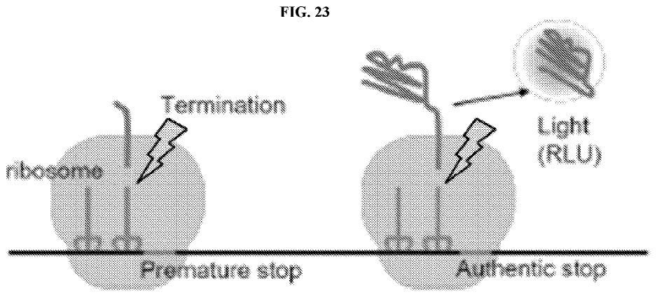

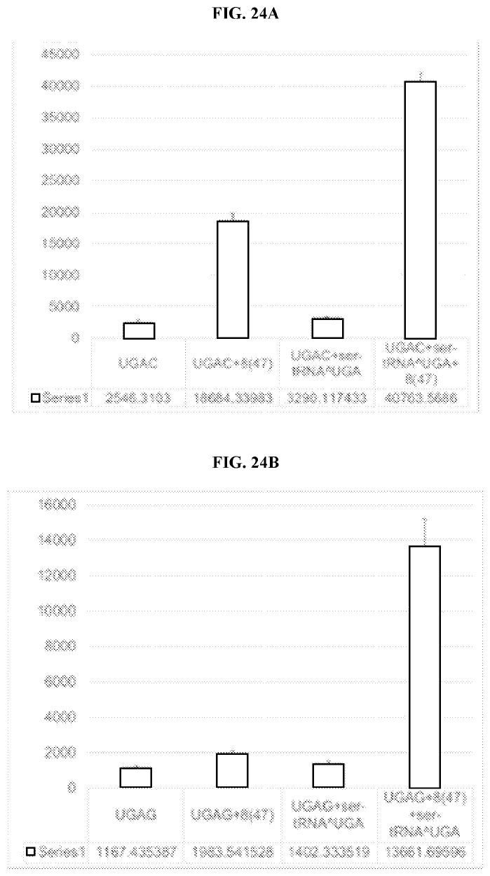

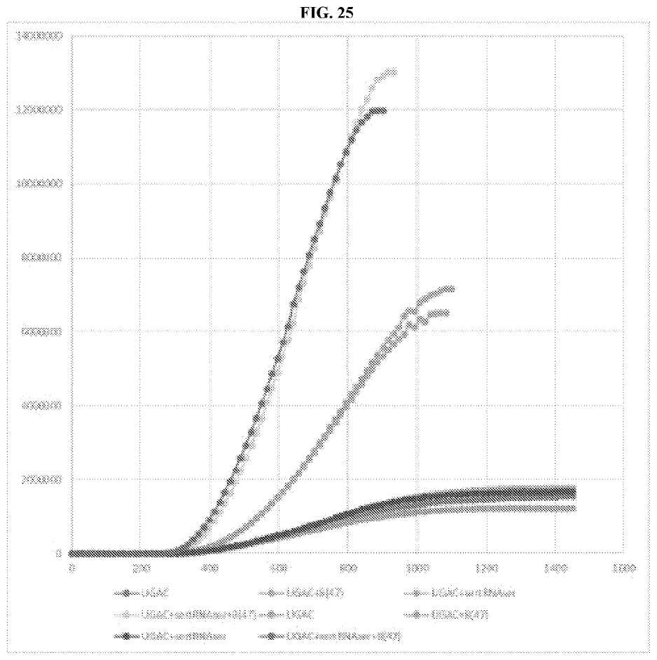

In one embodiment, the present invention contemplates a CFTR mRNA construct comprising a nonsense stop codon bound to a nucleic acid antisense +8(47) oligomer and a suppressor tRNA (e.g., ser-tRNA^{UGA}). See, FIG. 22. Data was collected by detecting luminescence generated by a full-length CFTR-nanoluciferase construct. See, FIG. 23. Three readthrough enhancer compositions were tested; i) 10 μM nucleic acid antisense +8(47) oligomer; ii) 0.5 M ser-tRNA^{UGA}; and iii) 10 μM nucleic acid antisense +8(47) oligomer +0.5 M ser-tRNA^{UGA} where mRNA constructs with both “weak” (UGAC) and “strong” (UGAG) nonsense stop codons were compared. See, FIGS. 24A and 24B. The raw data of FIG. 24 has been replotted in a line graph format. See, FIG. 25. These data clearly show that the combination of a nucleic acid antisense +8(47) oligomer and a ser-tRNA^{UGA} result in a synergistic improvement of nonsense stop codon readthrough. In particular, when compared to the nucleic acid antisense +8(47) oligomer alone, the UGAC codon readthrough is promoted by a two-fold factor and the UGAG codon readthrough is promoted by a seven-fold factor. Further, when compared to the ser-tRNA^{UGA} alone, the combination promotes UGAC codon readthrough by an approximate fifteen-fold factor and UGAG codon readthrough is promoted by an approximate nine-fold factor.

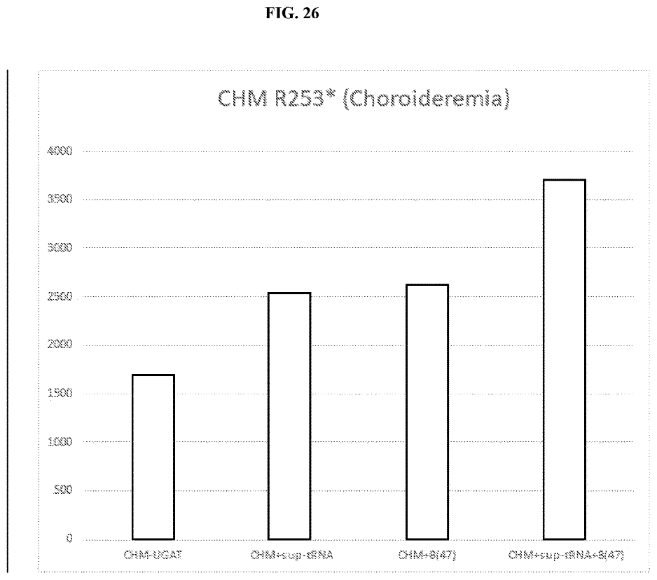

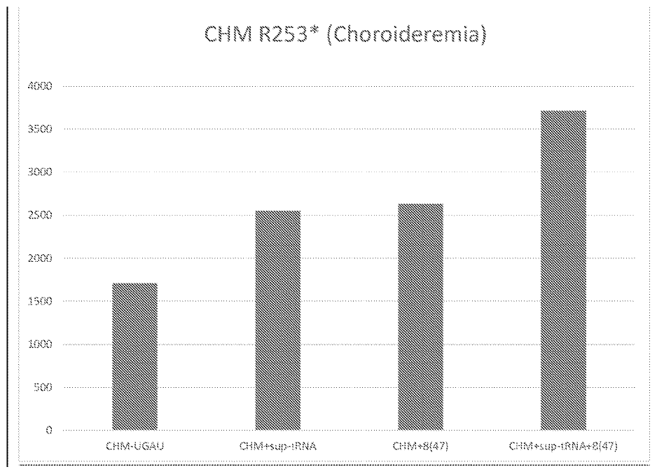

In one embodiment, the present invention contemplates an mRNA construct comprising a premature choroideremia stop codon (CHM R253*) bound to a nucleic acid antisense +8(47) oligomer and ser-tRNA^{UGA} The data show that a combination of a +8(47) oligomer and ser-tRNA^{UGA}, when compared to either compounds alone, promotes UGAU premature stop codon readthrough by an approximately 44%. See, FIG. 26.

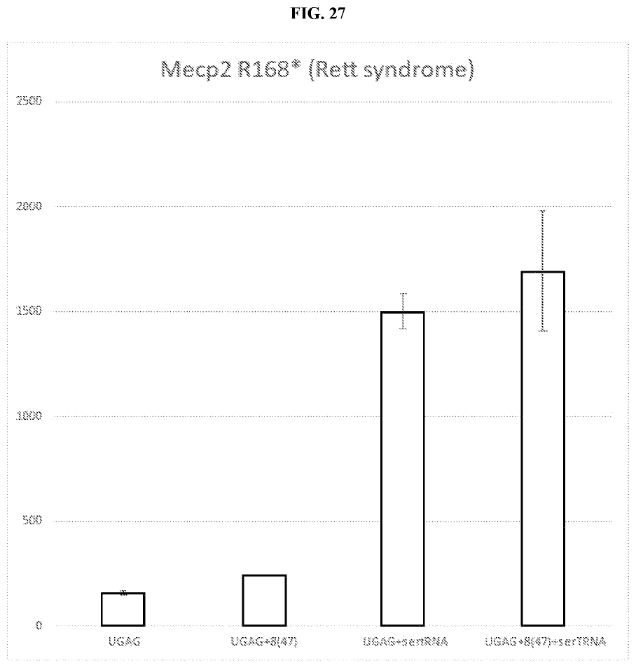

In one embodiment, the present invention contemplates an mRNA MECP2 (R168*) construct comprising a nonsense stop codon bound to a nucleic acid antisense +8(47) oligomer and ser-tRNA^{UGA}. The data show that a combination of a nucleic acid antisense +8(47) oligomer and ser-tRNA^{UGA}, when compared to the nucleic acid antisense +8(47) oligomer alone, promotes UGAG premature stop codon readthrough by a approximate seven-fold factor, while when compared to the ser-tRNA^{UGA} alone, promotes UGAG premature stop codon readthrough by approximately 13%. See, FIG. 27.

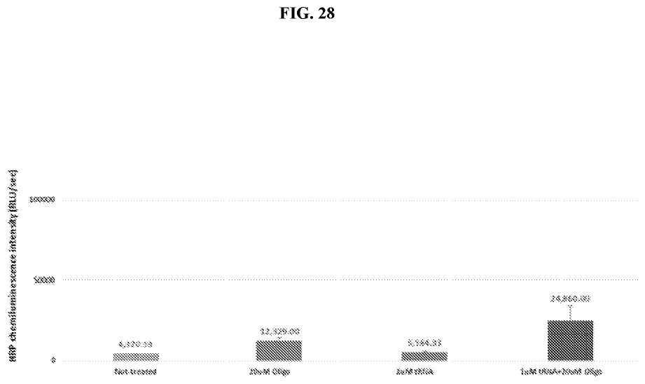

The data presented herein demonstrates successful readthrough of a CFTR-HRP (G542X) mRNA having a nonsense stop codon in FTR cells (~20,000 per well). In one embodiment, readthrough was promoted with: i) an nucleic acid antisense oligomer (20 μM); ii) a suppressor tRNA (1 μM); and iii) a combination of a nucleic acid antisense oligomer (20 μM) and suppressor tRNA (1 μM) in the presence of a transfection agent (e.g. MIR). Similar data was collected using a modified nucleic acid oligomer +8(47) 4/1 Fluoro-DNA: 5′-/52FU/CCA/i2FG/TCA/i2FG/TGT/i2FG/ATT/i2FG/CA/32FC/-3′ (data not shown). Data was measured via chemiluminescence using horseradish peroxidase (FRP) fused with a full-length CFTR construct. See, FIG. 28.

### VI. Positional Annealing Analysis of Nucleic Acid Antisense Oligomers

In one embodiment, the present invention contemplates a nucleic acid antisense oligomer having a plurality of nucleic acids comprising a 3′-terminal cytosine and a region complementary to at least positions +8-+20 of an mRNA that is downstream of a nonsense stop codon. In one embodiment, the mRNA comprises a guanosine positioned as the eighth nucleic acid from the first nucleic acid of the nonsense stop codon. In one embodiment, the nucleic acid antisense oligomer is a readthrough-antisense oligomer (R-ASO).

### A. Readthrough Dependence: R-ASO Annealing Position

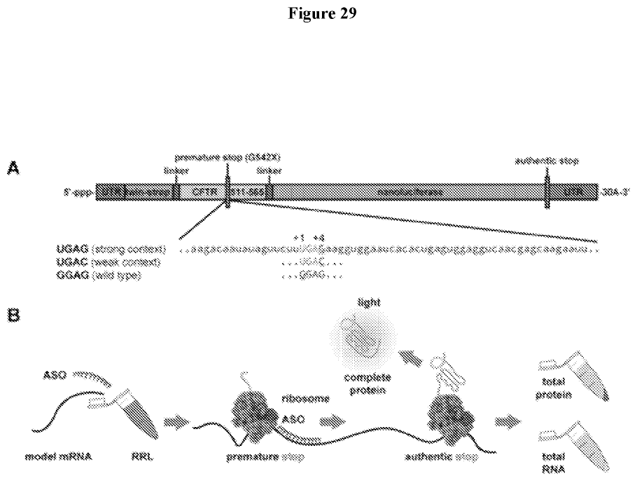

A reporter mRNA was developed in which translation of nanoluciferase requires readthrough of a CFTR G542X nonsense mutation, the most frequent nonsense mutation in cystic fibrosis patients. See, FIG. 29A. In the context of the CFTR mRNA coding sequence, the stop codon has a G542X mutation that is immediately followed by a guanosine nucleotide thereby forming the nonsense stop codon (UGA). This nonsense stop codon was then followed by a +4 purine (e.g., guanosine) to create a “strong” nonsense stop codon of UGAG, because the +4 purine forms stabilizing interactions with the 18S rRNA in the A site (Brown et al. 2015a; Matheisl et al. 2015a).

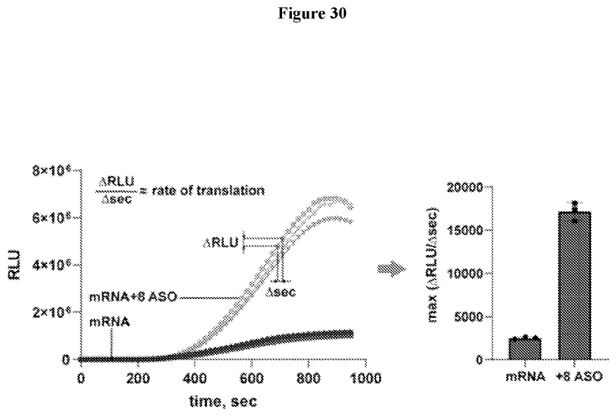

By contrast, translation termination can be orders-of-magnitude less efficient at a stop codon with a +4 pyrimidine (Wangen and Green 2020; Cridge et al. 2018; Brown et al. 1990; Jungreis et al. 2011). A reporter mRNA was also generated with a “weak” G542X nonsense UGA codon that was immediately followed by a +4 cytosine (i.e., UGAC) to readily detect readthrough. As a control to achieve maximal CFTR::nanoluciferase expression, a reporter mRNA was made with a wild-type CFTR G542 sense codon (i.e., GGAG), creating an intact open reading frame. Translation was monitored in rabbit reticulocyte lysates by recording luminescence in real time and the maximum change in relative light units was calculated to estimate the rate of translation. See, FIGS. 29B and 30.

The data show that wild-type CFTR G542 (GGAG) reporter mRNA was efficiently translated, producing an abundance of full-length CFTR::nanoluciferase fusion protein confirmed by western blot. By contrast, the nonsense stop codon-containing mRNAs were translated ~20-fold less efficiently than the wild-type reporter mRNA. The levels of protein detected by western blotting correlated with the translation efficiencies measured in the luminescence assay. Similar levels of nonsense stop codon-containing and glycine-encoding mRNAs were observed at the end of the translation reaction, indicating that the nonsense stop codons do not inhibit translation by promoting mRNA degradation. Thus, the nonsense stop codons inhibit translation with a low background of readthrough, suggesting that they are suitable for the studies of translation and nonsense stop codon readthrough. See, FIG. 31B.

To test the hypothesis that R-ASOs promote nonsense stop codon readthrough, a series of single stranded DNA R-ASOs (~20-nt, with similar predicted Tₘ) were designed which were complementary to a CFTR mRNA, each starting at a different position (e.g., +4 to +12) downstream of the nonsense stop codon. See, FIGS. 31A and 31C. Most R-ASOs in this series enhanced the readthrough of UGAC by less than 1.5-fold relative to background readthrough. Surprisingly, the +8 R-ASO, resulted in a 6.8-fold increase in the readthrough signal which was accompanied by a higher protein production than the other compounds. See, FIG. 31C, middle panel. Translation of the wild-type reporter mRNA was not affected by the R-ASOs, indicating that double-helical structures do not significantly block ribosomal translocation along mRNA. See, FIG. 31D. Varying the 5′-end of a +8 R-ASO revealed that twenty (20) nucleotides of complementarity most efficiently induced readthrough. See, FIG. 31E. Promotion of readthrough was also observed to be dose-dependent, with 150 nM +8 R-ASO (e.g., a 5-fold excess over mRNA) sufficient to promote readthrough. See, FIG. 31F.

The same promotional effects were observed for mRNAs bearing a 5′ cap and a longer poly(A)-tail, characteristic features of cellular mRNAs. See, FIG. 32. These results indicate that a +8 R-ASO promotes efficient and specific readthrough of an mRNA with a nonsense stop codon without modifying the expression of an mRNA with a wild-type sense stop codon.

Analysis of mRNA integrity revealed an additional mRNA fragment appearing upon incubation with above R-ASO series, suggesting partial mRNA degradation. See, FIG. 31C, (lower panel, black arrow). The fragmentation could be attributed to the cleavage of mRNA by RNase H, whose activity in RRL was previously reported. (Cazenave et al. 1993). Indeed, when the mRNA was treated with a +8 DNA R-ASO and recombinant E. coli RNase H, translation was substantially decreased due to mRNA degradation. See, FIGS. 33A&B. Notably, the low levels of DNA-induced fragmentation of mRNA in the RRL do not correlate with readthrough efficiency, indicating that mRNA degradation does not substantially interfere with R-ASO induced readthrough.

B. Synergistic Readthrough with +8 R-ASO Combined with G418 or stRNAs

After establishing the readthrough conditions for a UGAC nonsense stop codon termination context, a disease-causing CFTR G542X mRNA with a UGAG nonsense stop codon was tested. Readthrough on the CFTR G542X mRNA with a UGAG nonsense stop codon was slightly promoted by R-ASOs, suggesting that a nucleic acid antisense oligomer (R-ASO) has reduced efficacy to outcompete eRF1 binding and/or promote readthrough translation at a strong nonsense stop codon. See, FIG. 34B (3B)

It has been previously reported that combinations of readthrough-promoting molecules can synergistically increase readthrough efficiency. Zhang et al. 2018; Xue et al. 2014; Martins-Dias and Romão 2021. Testing was performed to determine if a +8 R-ASO can synergize with either G418 or a UGA-suppressor tRNA (e.g., ser-tRNA^{UGA}) to enhance readthrough. See, FIG. 35. G418 (0.5 μM) was observed to achieve the highest readthrough of a UGAC nonsense stop codon construct. See, FIG. 34D. G418 or Ser-tRNA^{UGA} alone partially stimulated (e.g., 3.8-fold and ~1.5-fold, respectively) readthrough of the weak CFTR UGAC nonsense stop codon but not of a strong CFTR UGAG nonsense stop codon. See, FIGS. 34A & 34B. A synergistic readthrough response was observed when +8 R-ASO was combined with either G418 or ser-tRNA^{UGA} of both weak (UGAC) and strong (UGAG) nonsense stop codons, exceeding the sum of individual effects of readthrough compounds. Synergy was most pronounced for the weak UGAC nonsense stop codon (15- to 20-fold increase), producing near wild-type levels of the reporter protein. For the UGAG nonsense stop codon, readthrough promotion synergy was observed between +8 R-ASO and G418 (~5-fold) or Ser-tRNA^{UGA} (~10-fold). Concomitantly, reporter protein expression was restored to ~25% the level produced by a wild-type CFTR sense reporter. See, FIGS. 34A & 34B.

It has been reported that aminoglycosides are toxic in vivo at the concentrations at which they can promote nonsense stop codon readthrough alone. (Peloquin et al. 2004). Indeed, in a control experiment, G418 inhibited translation of a wild-type-like GGAG mRNA, in keeping with its indiscriminate miscoding effects. See, FIG. 34C. Measuring a G418 dose response for a UGAC nonsense stop codon construct revealed that at least a two-fold lower G418 concentration could achieve the maximum readthrough effect with R-ASO than without it. See, FIG. 34D. This synergy may improve the therapeutic potential of combination therapies due to reduced non-specific readthrough and lowered toxic side effects.

### C. +4, +5, +6, +7, +8 and +9 R-ASO Inhibition of Translation Termination

The Termi-luc assay was used to measure the kinetics of full-protein release from pretermination complexes. (Susorov et al. 2020). Purified 80S ribosomes were paused at a weak CFTR UGAC nonsense stop codon featuring a P-site tRNA-nanoluciferase. See, FIG. 36A. Addition of a ternary eRF1*eRF3*GTP termination complex results in the release of nanoluciferase from the ribosome, yielding an increase in luminescence. See, FIG. 36B; and (Susorov et al. 2020).

Preincubation of the 80S complex with R-ASOs resulted in varying termination rates. Termination was: i) significantly inhibited by R-ASOs that anneal at positions +4 through +8; ii) partially inhibited by R-ASOs that anneal at position +9; and iii) unaffected by R-ASOs that anneal at positions +10 through +12. See, FIG. 36B. These results suggest that translation termination inhibition depends on the proximity of a R-ASO/mRNA duplex to the A site and/or ribosomal mRNA tunnel entry.

The data show that a +8 R-ASO has the greatest efficiency to promote nonsense stop codon readthrough in the RRL system. See, FIG. 31C; and FIG. 37A. Although it is not necessary to understand the mechanism of an invention, it is believed that a translating ribosome may dissociate from an annealed R-ASO close to the A site (e.g., between positions +4 to +7), thus preventing translation termination inhibition. In summary, the ability of a +8 R-ASO to promote nonsense stop codon readthrough is at least in part due to translation termination inhibition. The other tested oligonucleotides that inhibit termination (+4, +5, +6, +7 and +9) also have the potential to induce readthrough if tested under different conditions, using the same mechanism (termination inhibition) as the +8 R-ASO.

### D. Chemically Modified +8 R-ASOs

The effects of R-ASO nucleotide modifications were tested for their effects on nonsense stop codon readthrough and overall mRNA stability. A panel +8 R-ASOs were designed, each with similar nucleic acid sequences but having different patterns of nucleotide modifications. See, FIGS. 37A & 37B; and (Roberts et al. 2020).

Using the weak UGAC Thermi-Luc reporter system, the data showed that readthrough activity was influenced by both the position and type of nucleic acid modification. R-ASOs with chemical modifications throughout the 5′ terminal region retained readthrough activity (e.g., oligo 9). For example, some 2′-deoxy and 2′-fluoro nucleotides are well tolerated (e.g. oligos 1-11). By contrast, chemical modifications primarily within the 3′ terminal region differentially modulated readthrough efficiency: i) extensive modifications reduced readthrough efficiency (e.g., oligo 14); ii) 2′-locked and 2′-fluoro modifications were well tolerated; and iii) 2′-Ome modifications partially reduced readthrough promotion (e.g., oligos 8, 4 and 12). Mismatched nucleotides and dN/2′F oligonucleotides were not active (e.g., oligos 15 & 30). Phosphorothioate (PS) linkages decreased readthrough promotion depending on the number of substitutions (cf, oligos 2 with 11). For example, saturation of +8 R-ASOs with PS completely inhibited readthrough promotion with mRNAs having either UGAC or GGAG nonsense stop codons(e.g., oligos 25-28). The inhibitory effect of phosphorothioate on translation was not sequence-specific, as the mismatched PS oligo 31 also inhibited translation, consistent with the reported non-specific in vivo toxicity of highly PS-modified oligonucleotides. See, FIG. 37A; and (Anderson et al. 2021).

Overall, these data reveal that promotion of nonsense codon readthrough with chemically modified R-ASOs is positionally dependent. For example, when only the 5′-end is heavily chemically modified and the 3′end is not, or lightly, modified a strong promotion of readthrough activity is consistently observed. Although it is not necessary to understand the mechanism of an invention, it is the believed that since a 3′-end of a R-ASO is likely positioned at the mRNA entrance and may interact with the ribosome, it is more sensitive to the types and positions of modified nucleotides than the 5′-end.

E. Broad-Based mRNA Targeting By +8 R-ASOs

The data presented herein demonstrates that a specific R-ASO design may promote readthrough in a plurality of disease-causing mRNAs containing a nonsense stop codon.

1. Chimeric MCEP2/CFTR mRNA

A reporter mRNA was designed that encodes a Rett syndrome MECP2 nonsense stop codon comprising an R168X mutation. See, FIG. 38A. As the R168X nonsense stop codon is immediately followed by a +4 G nucleotide it is believed to be a strong stop codon. For example, a +8 Mcep2 R-ASO failed to induce readthrough in the presence or absence of G418 or Ser-tRNA^{UGA}. See, FIGS. 38A and 38B.

A chimeric MCEP2 mRNA was designed by replacing two regions downstream of the nonsense stop codon with sequences from a CFTR mRNA. In the first construct, nucleotides +4 to +7 (i.e., immediately after the nonsense codon) were replaced. In the second construct, nucleotides +8 to +27 were replaced. See, FIG. 38C. The data shows that the second construct substantially improved the readthrough activity of the chimeric MECP2 mRNA (e.g., ~4.7-fold) as compared to the mutated MECP2 mRNA. See, FIG. 38C. This observation is consistent with previous studies showing that mRNA nucleotides downstream of the stop codon can affect termination and readthrough. (Cridge et al. 2018; Anzalone et al. 2019; Biziaev et al. 2022).

### 2. Readthrough Sensitivity Determination

The data presented herein demonstrate that sequence properties of the +8 R-ASO-binding region of an mRNA confer readthrough sensitivity. The +8 position was likely involved as seen from the above register shifting and R-ASO modification experiments. See, FIGS. 31C and 37A. The Thermi-luc reporter mRNA was modified by substituting the +8 G with either A, C, or U. These variants were then tested for the ability of complementary or mismatched +8 R-ASOs to promote readthrough. The data show that readthrough was promoted by +8 R-ASOs that base pair at the +8 position. See, FIG. 31D. Furthermore, readthrough efficiency was much higher with +8 G than any other nucleotide. These results indicate that R-ASO-promoted nonsense stop codon readthrough requires base-pair interactions near the mRNA tunnel and correlate with +8 nucleotide identity reported for the readthrough efficiency in mammalian cells. (Cridge et al. 2018).

3. Positional mRNA G/C Content Evaluation

The positions of purine and pyrimidine nucleotides were randomized in a CFTR +8-+27 mRNA region (e.g., RYRRRRYYRYRYYRRRYRRRR, where R denotes purines and Y pyrimidines) while retaining similar predicted stability of the corresponding duplexes (as measured by Tₘ). Three specific randomized sequences were generated, RND1-3, and readthrough was compared to a wild type CFTR mRNA. The data showed that RND2 was nearly inactive, RND1 was slightly less active, and RND3 demonstrated higher readthrough efficiency. The inactive RND2 reporter had a +8 G-to-A replacement, resembling the inhibitory effect of a +8 substitution in the CFTR mRNA shown above. FIG. 38D. Most notably, the total G nucleotide content at +8 and nearby positions was higher in readthrough-prone sequences (wild type CFTR, RND1, and RND3) and correlated with an increase in R-ASO-induced readthrough.

This observation suggests that the lack of +8 R-ASO promoted readthrough of MECP2 mRNA with an R168X nonsense stop codon may be due to the presence of an A at position +8 in addition to a single guanosine among the five initial positions. See, FIG. 38A. For example, five variant MECP2 reporter mRNAs with nonsense stop codons were constructed, each with a +8 G, and increasing sequential additional G nucleotides adjacent to the +8 position. The overall G/C-content in the MECP2 mRNA was preserved by converting downstream C nucleotides to U nucleotides. A single +8 G substitution enabled R-ASO-induced readthrough in the presence of Ser-tRNA^{UGA} (~2-fold relative to tRNA alone). R-ASO-induced readthrough was most efficient on a variant reporter with four sequential G nucleotides from +8-+11 (~3-fold greater than tRNA alone. See, FIG. 38F.

These findings indicate that R-ASO-induced readthrough depends on the identity of mRNA nucleotides in the R-ASO-binding region. Although it is not necessary to understand the mechanism of an invention, it is believed that disease-causing mRNAs having nonsense stop codons and guanosines at +8 and neighboring positions are most susceptible to R-ASO promoted readthrough.

### 4. +8 R-ASO Consensus Sequence

To evaluate the number of the nonsense stop codon contexts potentially prone to the ASO-dependent readthrough, +8-+27 mRNA sequences following nonsense stop codons were complied from all human mRNAs as annotated in the LOVD3 database. (Fokkema et al. 2011, 2021). A first mRNA consensus sequence was generated using an alignment analysis of the readthrough contexts of CFTR, RND1, RND2, Var 4 and Var 5. See, FIG. 38G. A second mRNA consensus sequence was generated using an alignment analysis of the readthrough contexts of CFTR, RND1, RND2, Var 1, Var 4 and Var 5. See, FIG. 38H.

A simplified query was generated from these consensus sequences with G nucleotides at positions +8 and/or +11 and was entered onto the LOVD3 database. Of the 22,343 found nonsense contexts in 3279 genes, 798 were nonsense mutations (3.5%) in 464 human genes. FIGS. 38G & 38H. Although it is not necessary to understand that mechanism of an invention, it is believed that these 798 mRNAs with nonsense mutations and G nucleotides at positions +8 and +11 may be amenable to +8 R-ASO treatment. For example, of these 798 mRNAs with nonsense mutations and G nucleotides at positions +8 and +11, 709 (>88%) are reported to be pathogenic/likely pathogenic in LOVD3 database.

### VII. Practical Applications of R-ASO Technology

The data presented herein demonstrate that R-ASOs that anneal to an mRNA downstream of a nonsense stop codon can promote readthrough and inhibition of premature translation termination. The readthrough efficiency depends on the position and nucleotide identities of the R-ASO*mRNA duplex. For example, in both native CFTR G542X and mutated MECP2 mRNA contexts, R-ASOs binding at +8 position promoted efficient readthrough. Although it is not necessary to understand the mechanism of an invention, it is believed that the promoted nonsense stop codon readthrough is due to an interaction of the R-ASO*mRNA duplex with the ribosomal mRNA entry channel, which prevents mRNA from being pulled into the A site for stop-codon recognition by eRF1 (Brown et al. 2015a; Matheisl et al. 2015a). See, FIGS. 39A & 39C. Indeed, structural and biochemical studies have identified the +8 position (counting from the first nucleotide of the nonsense codon) at the entry of the bacterial ribosome tunnel. (Takyar et al. 2005; Amiri and Noller 2019b, 2019a). For clarity, it should be noted that these references refer to this position as +11 because they count from the first nucleotide of the P-site codon, not the nonsense stop codon. (Amiri and Noller 2019b)).

The mRNA tunnels of the bacterial and mammalian ribosomes are structurally similar. See, FIG. 39B. The +8 position may, therefore, be the first available position to form the R-ASO*mRNA duplex and sterically block termination. This idea is supported by kinetic measurements of protein release, showing that duplexes at +8 positions strongly inhibit termination, whereas those binding farther from the tunnel entry fail to inhibit termination. See, FIG. 36B. mRNA secondary structures downstream of a nonsense codon also promote readthrough and have been proposed to do so by sterically inhibiting termination. (Manjunath et al. 2022; Anzalone et al. 2019).

R-ASO-induced readthrough was more effective on a weak UGAC nonsense stop codon context than on a strong UGAG nonsense stop codon, consistent with the importance of the +4 nucleotide in stop-codon termination and readthrough. (Cridge et al. 2018; Wangen and Green 2020; Brown et al. 1990). R-ASOs nevertheless induced readthrough on the strong nonsense stop codon in the presence of G418 or suppressor tRNA, which alone were inefficient. See, FIG. 35B. These results highlight the fact that stop codon readthrough is a competitive process in which termination and elongation work against each other. Thus, tuning translation termination down with an R-ASO and fueling elongation up with G418 or a suppressor tRNA allows for efficient stop codon readthrough.

Interestingly, R-ASOs annealing to +4-+7 inhibited isolated termination reaction along with +8-R-ASO, but were inactive in promoting readthrough in RRL See, FIGS. 36B and 31C. This can be explained by the fact that in a full translation system, translocation occurs before the ribosome stumbles upon the premature stop codon. Translocation was shown to be the primary source of the ribosomal helicase activity (Takyar et al. 2005; Amiri and Noller 2019b, 2019a), which could unwind duplexes getting too close to ribosome. An efficient helicase activity of elongating ribosomes in RRL is supported by the fact that R-ASOs do not affect expression of the control wild-type CFTR-construct (GGAG).

The present findings reveal that the downstream context at the +8 mRNA position strongly affects the sensitivity to readthrough. At +8 position, guanosine is susceptible to high readthrough in otherwise identical constructs, followed by A and by both pyrimidines. See, FIG. 38D. While the preference for the G can be explained by its ability to stabilize a duplex, the inability of the reverse pair (+8 C:G) to induce readthrough suggests that specific interactions of the helix with the ribosome may play a role. (Manosas et al. 2010; Borisova et al. 1993).

In the positions downstream from the +8 nucleotide, an increasing G/C-content improves the readthrough. See, FIG. 38F. While the local helix stability appears to be a factor of readthrough efficiency, additional effects, such as interactions with the ribosome components, may also be involved.

Using different nucleotide chemical modifications, the 5′-portion of an +8 R-ASO appears to promote readthrough when partly or completely chemically modified, whereas chemical modification of the 3′-portion loses readthrough promotion activity. This asymmetry, together with the sensitivity of readthrough to the mRNA context at +8 position, suggests readthrough promotion may be mediated by an interaction between the R-ASO*mRNA duplex and mRNA tunnel entry.

It has been reported that RNA-DNA duplexes have unique properties, which differentiate them from both RNA-RNA and DNA-DNA duplexes. In particular, RNA-DNA duplexes can adopt intermediate A/B-form conformations upon interactions with surrounding molecules (Horton and Finzel 1996). Such conformation(s) may facilitate the installation of the R-ASO at the mRNA entry channel to inhibit translation termination at the upstream nonsense codon.

In one embodiment, nucleic acid antisense oligonuleotides are crosslinked to the “helper” molecules (e.g., stRNAs, aminoglycosides, oligomucleotides and linkers). See, FIGS. 40A-D. These “helper’ molecules are believed to further improve the readthrough due to a spatial proximity that allows them to act on the same mRNA-ribosome complex. See, FIGS. 40 E & F. Such crosslinked helper oligonucletides bind on the mRNA downstream of the antisense to improve overall binding which may reduce the therapeutic dose of the nucleic acid antisense oligomer.

### A. R-ASO Embodiments

Based upon the above constructed mRNA consensus sequences, consensus R-ASO designs can be defined by a conservative complementarity conversion analysis. Briefly, various combinations of the nucleic acids most frequently found in the +8-+27 mRNA regions were queried in the LOVD3 database for aligning base pairs. See, Schnieder et al., Applied Bioinfom. 2002.

1. mRNA +8G/+11G/+14T/+17C/+23A Query

The LOVD3 database was queried for mRNA sequences that contain a nonsense stop codon with +8G/+11G/+14T/+17C/+23A positions downstream of the first nucleic acid of the nonsense stop codon. This query returned twenty matching mRNAs from which complementary R-ASOs were generated. See, Table 2.

TABLE 2
+8G/+11G/+14T/+17C/+23A Query Database Hits

| | NSC | +8-+27 mRNA sequence | SEQ | R-ASO | SEQ |
|---|---|---|---|---|---|
| Gene | # | 5′-3′ direction | ID | 5′-3′ direction | ID |
| PKP2 | 79 | GCAGTGTTCCTGAGTATGTC | 17 | GACATACTCAGGAACACTGC | 36 |
| CNGA3 | 344 | GGCGCCTCTCCAGGAAGTAC | 18 | GTACTTCCTGGAGAGGCGCC | 37 |
| PKP2 | 79 | GCAGTGTTCCTGAGTATGTC | 19 | GACATACTCAGGAACACTGC | 38 |
| USH2A | 1838 | GAGGAATCCCACAGGAACTG | 20 | CAGTTCCTGTGGGATTCCTC | 39 |
| EVC | 622 | GCGGCCTCACTGAAGAGTCC | 21 | GGACTCTTCAGTGAGGCCGC | 40 |
| NTRK1 | 166 | GCGGAGTGCCTGAACAGAAG | 22 | CTTCTGTTCAGGCACTCCGC | 41 |
| KIF11 | 53 | GAGGATTGGCTGACAAGAGC | 23 | GCTCTTGTCAGCCAATCCTC | 42 |
| TCF4 | 567 | GGAGGATGGCCAACAATGCC | 24 | GGCATTGTTGGCCATCCTCC | 43 |
| RP1L1 | 424 | GGGGACTGGCCCAGCACGTC | 25 | GACGTGCTGGGCCAGTCCCC | 44 |
| PMEL | 516 | GCGGGCTGCCCAAGGAAGCC | 26 | GGCTTCCTTGGGCAGCCCGC | 45 |
| PKD1 | 2528 | GCAGCCTCTCCAGCTACGGA | 27 | TCCGTAGCTGGAGAGGCTGC | 46 |
| LTBP4 | 1296 | GCTGCGTCTCCAACGAGAGC | 28 | GCTCTCGTTGGAGACGCAGC | 47 |
| IGSF1 | 921 | GGAGTGTCTCAGGGAACTCA | 29 | TGAGTTCCCTGAGACACTCC | 48 |
| GFM2 | 117 | GAGGCATTACTATTCAATCA | 30 | TGATTGAATAGTAATGCCTC | 49 |
| F8 | 199 | GGAGTCTGGCCAAGGAAAAG | 31 | CTTTTCCTTGGCCAGACTCC | 50 |
| DSP | 682 | GCAGGATGACTCTCAAAAAC | 32 | GTTTTTGAGAGTCATCCTGC | 51 |
| CHD7 | 2024 | GTCGAATGCCCGTCAAGCCA | 33 | TGGCTTGACGGGCATTCGAC | 52 |
| CERKL | 362 | GATGGATGTCCCCTAACCAA | 34 | TTGGTTAGGGGACATCCATC | 53 |
| BCOR | 1161 | GACGAGTCTCTAAAGATGAC | 35 | GTCATCTTTAGAGACTCGTC | 54 |

NSC # = Nonsense stop codon mRNA position.

In one embodiment, the present invention contemplates a nucleic acid antisense oligomer comprising, or consisting of, a plurality of nucleic acids having the sequence of:

5′-A₂₀-A₁₉-A₁₈-A₁₇-A₁₆-A₁₅-A₁₄-A₁₃-A₁₂-A₁₁-A₁₀-A₉-A₈-
A₇-A₆-A₅-A₄-A₃-A₂-A₁-3′

wherein, A₂₀ is any nucleic acid, A₁₉ is any nucleic acid, A₁₈ is any nucleic acid, A₁₇ is any nucleic acid, A₁₆ is T, A₁₅ is any nucleic acid, A₁₄ is any nucleic acid, A₁₃ is any nucleic acid, A₁₂ is any nucleic acid, A₁₁ is any nucleic acid, A₁₀ is G, A₉ is any nucleic acid, A₈ is any nucleic acid, A₇ is A, A₆ is any nucleic acid, A₅ is any nucleic acid, A₄ is C, A₃ is any nucleic acid, A₂ is any nucleic acid and A₁ is C.
2. mRNA +8G/+II G/+14T/+23A Query

The LOVD3 database was queried for mRNA sequences that contain a nonsense stop codon with +8G/+11G/+14T/+23A positions downstream of the first nucleic acid of the nonsense stop codon. This query returned sixty-six matching mRNAs from which complementary R-ASOs were generated. See, Table 3.

TABLE 3
+8G/+11G/+14T/+23A Database Hits

| | NSC | +8-+27 mRNA sequence | SEQ | R-ASO | SEQ |
|---|---|---|---|---|---|
| Gene | # | 5′-3′ direction | ID | 5′-3′ direction | ID |
| PKP2 | 79 | GCAGTGTTCCTGAGTATGTC | 55 | GACATACTCAGGAACACTGC | 120 |
| SMAD4 | 398 | GGTGCCTTAGTGACCACGCG | 56 | CGCGTGGTCACTAAGGCACC | 121 |
| CNGA3 | 344 | GGCGCCTCTCCAGGAAGTAC | 57 | GTACTTCCTGGAGAGGCGCC | 122 |
| PKP2 | 79 | GCAGTGTTCCTGAGTATGTC | 58 | GACATACTCAGGAACACTGC | 123 |
| MITF | 315 | GAAGATTTAACATAAATGAC | 59 | GTCATTTATGTTAAATCTTC | 124 |
| USH2A | 1838 | GAGGAATCCCACAGGAACTG | 60 | CAGTTCCTGTGGGATTCCTC | 125 |
| GCDH | 352 | GCCGCTTGAAGGACCAGGAC | 61 | GTCCTGGTCCTTCAAGCGGC | 126 |
| EVC | 622 | GCGGCCTCACTGAAGAGTCC | 62 | GGACTCTTCAGTGAGGCCGC | 127 |
| SBF2 | 1196 | GAGGATTCCATGGGAAGGGA | 63 | TCCCTTCCCATGGAATCCTC | 128 |
| NTRK1 | 166 | GCGGAGTGCCTGAACAGAAG | 64 | CTTCTGTTCAGGCACTCCGC | 129 |
| NR0B1 | 197 | GCTGCTTTTGCGGTGAAGAC | 65 | GTCTTCACCGCAAAAGCAGC | 130 |
| KIF11 | 53 | GAGGATTGGCTGACAAGAGC | 66 | GCTCTTGTCAGCCAATCCTC | 131 |
| GBA2 | 745 | GTAGTGTTATGTCTGACCAG | 67 | CTGGTCAGACATAACACTAC | 132 |
| ERCC6 | 947 | GGAGAATAGGCCAGAAGAAG | 68 | CTTCTTCTGGCCTATTCTCC | 133 |
| DLG4 | 150 | GCCGCCTCAGGGTCAACGAC | 69 | GTCGTTGACCCTGAGGCGGC | 134 |
| CRB1 | 120 | GAGGTATTTGCCATCAGGAC | 70 | GTCCTGATGGCAAATACCTC | 135 |
| BBS2 | 234 | GGAGAATTAAATCGAAAAAT | 71 | ATTTTTCGATTTAATTCTCC | 136 |
| BBS1 | 393 | GTGGCCTGATCATCAAGATC | 72 | GATCTTGATGATCAGGCCAC | 137 |
| TCF4 | 567 | GGAGGATGGCCAACAATGCC | 73 | GGCATTGTTGGCCATCCTCC | 138 |
| TAZ | 195 | GGCGCCTGATTGCTGAGTGT | 74 | ACACTCAGCAATCAGGCGCC | 139 |
| SYNGAP1 | 1219 | GGAGGCTGCTGTCCCAGGAA | 75 | TTCCTGGGACAGCAGCCTCC | 140 |
| SMAD6 | 14 | GTCGTGTGGTCCCCGACCGG | 76 | CCGGTCGGGGACCACACGAC | 141 |
| SGCB | 28 | GGAGTGTCAATAAAGAGCAC | 77 | GTGCTCTTTATTGACACTCC | 142 |
| RP1L1 | 424 | GGGGACTGGCCCAGCACGTC | 78 | GACGTGCTGGGCCAGTCCCC | 143 |
| RBP4 | 23 | GCAGCTTCCGAGTCAAGGAG | 79 | CTCCTTGACTCGGAAGCTGC | 144 |
| RAD50 | 1014 | GGTGGCTACAAGATAACCTT | 80 | AAGGTTATCTTGTAGCCACC | 145 |
| PTCH1 | 184 | GCCGTGTCCATGTATACATG | 81 | CATGTATACATGGACACGGC | 146 |
| PMEL | 516 | GCGGGCTGCCCAAGGAAGCC | 82 | GGCTTCCTTGGGCAGCCCGC | 147 |
| PKD2 | 48 | GGGGCCTGGAGATCGAGATG | 83 | CATCTCGATCTCCAGGCCCC | 148 |
| PKD1 | 3114 | GCCGCTTCAAGTACGAGATC | 84 | GATCTCGTACTTGAAGCGGC | 149 |
| PKD1 | 2528 | GCAGCCTCTCCAGCTACGGA | 85 | TCCGTAGCTGGAGAGGCTGC | 150 |
| PAX3 | 40 | GCGGTGTTTTTATCAACGGC | 86 | GCCGTTGATAAAAACACCGC | 151 |
| PALB2 | 650 | GCTGTATTTTTCCAGAGGAA | 87 | TTCCTCTGGAAAAATACAGC | 152 |
| OPA1 | 902 | GGCGATTAGAGAAAAATGTT | 88 | AACATTTTTCTCTAATCGCC | 153 |
| NR0B1 | 130 | GCTGCTTTTGTGGTGAAGAC | 89 | GTCTTCACCACAAAAGCAGC | 154 |
| NLRP1 | 132 | GAAGGGTTTTGAGACAGCTG | 90 | CAGCTGTCTCAAAACCCTTC | 155 |
| MNX1 | 290 | GGCGGATGAAATGGAAACGC | 91 | GCGTTTCCATTTCATCCGCC | 156 |
| MITF | 214 | GAAGATTTAACATAAATGAC | 92 | GTCATTTATGTTAAATCTTC | 157 |
| MBD5 | 25 | GTCGTGTGGATCAAAATGGA | 93 | TCCATTTTGATCCACACGAC | 158 |
| LTBP4 | 1296 | GCTGCGTCTCCAACGAGAGC | 94 | GCTCTCGTTGGAGACGCAGC | 159 |
| KCNV2 | 177 | GCCGCTTCCTGGAGGAGCTG | 95 | CAGCTCCTCCAGGAAGCGGC | 160 |
| KCNJ10 | 26 | GGAGAGTCCTGACAAAAGAT | 96 | ATCTTTTGTCAGGACTCTCC | 161 |
| ITGB2 | 734 | GGCGCTTTGAGAAGGAGAAG | 97 | CTTCTCCTTCTCAAAGCGCC | 162 |
| IGSF1 | 921 | GGAGTGTCTCAGGGAACTCA | 98 | TGAGTTCCCTGAGACACTCC | 163 |
| HSD11B2 | 261 | GCTGCTTCAAGACAGAGTCA | 99 | TGACTCTGTCTTGAAGCAGC | 164 |
| GMPPB | 264 | GCAGCATTGGCCCCAATGTG | 100 | CACATTGGGGCCAATGCTGC | 165 |
| GLA | 102 | GCAGACTTCAGGCAGACCCT | 101 | AGGGTCTGCCTGAAGTCTGC | 166 |
| GFM2 | 117 | GAGGCATTACTATTCAATCA | 102 | TGATTGAATAGTAATGCCTC | 167 |
| F8 | 199 | GGAGTCTGGCCAAGGAAAAG | 103 | CTTTTCCTTGGCCAGACTCC | 168 |
| DSP | 682 | GCAGGATGACTCTCAAAAAC | 104 | GTTTTTGAGAGTCATCCTGC | 169 |
| DMD | 548 | GCTGGGTTCTTTTACAAGAC | 105 | GTCTTGTAAAAGAACCCAGC | 170 |
| D2HGDH | 169 | GCTGCGTCCTGGAGGAGCTG | 106 | CAGCTCCTCCAGGACGCAGC | 171 |
| CUL3 | 436 | GGAGACTTCTCACAAATAAA | 107 | TTTATTTGTGAGAAGTCTCC | 172 |
| CNOT3 | 694 | GGCGATTCCACACCAAGTAC | 108 | GTACTTGGTGTGGAATCGCC | 173 |
| CHD7 | 2024 | GTCGAATGCCCGTCAAGCCA | 109 | TGGCTTGACGGGCATTCGAC | 174 |
| CHD3 | 457 | GCCGCGTATGCAAGGACGGC | 110 | GCCGTCCTTGCATACGCGGC | 175 |
| CHD3 | 1137 | GGGGCCTGGGCATCAATCTG | 111 | CAGATTGATGCCCAGGCCCC | 176 |
| CERKL | 362 | GATGGATGTCCCCTAACCAA | 112 | TTGGTTAGGGGACATCCATC | 177 |
| CD3D | 93 | GCTGTGTGGAGCTGGATCCA | 113 | TGGATCCAGCTCCACACAGC | 178 |
| BRWD3 | 729 | GAAGAGTGGTGGTCAATGAA | 114 | TTCATTGACCACCACTCTTC | 179 |
| BCOR | 1161 | GACGAGTCTCTAAAGATGAC | 115 | GTCATCTTTAGAGACTCGTC | 180 |
| APC | 848 | GAAGTTTGGAGAGAGAACGC | 116 | GCGTTCTCTCTCCAAACTTC | 181 |
| ANK1 | 1252 | GGCGCCTGCGCTGCTACTGC | 117 | GCAGTAGCAGCGCAGGCGCC | 182 |
| ALPI | 118 | GCGGGGTCAAGGCCAACTTC | 118 | GAAGTTGGCCTTGACCCCGC | 183 |
| ABCA4 | 2104 | GCCGCATGCTGTGGAACGTC | 119 | GACGTTCCACAGCATGCGGC | 184 |

NSC # = Nonsense stop codon mRNA position.

In one embodiment, the present invention contemplates a nucleic acid antisense oligomer comprising, or consisting of, a plurality of nucleic acids having the sequence of:

5′-B₂₀-B₁₉-B₁₈-B₁₇-B₁₆-B₁₅-B₁₄-B₁₃-B₁₂-B₁₁-B₁₀-B₉-B₈-
B₇-B₆-B₅-B₄-B₃-B₂-B₁-3′

wherein, B₂₀ is any nucleic acid, B₁₉ is any nucleic acid, B₁₈ is any nucleic acid, B₁₇ is any nucleic acid, B₁₆ is T, B₁₅ is any nucleic acid, B₁₄ is any nucleic acid, B₁₃ is any nucleic acid, B₁₂ is any nucleic acid, B₁₁ is any nucleic acid, B₁₀ is any nucleic acid, B₉ is any nucleic acid, B₈ is any nucleic acid, B₇ is A, B₆ is any nucleic acid, B₅ is any nucleic acid, B₄ is C, B₃ is any nucleic acid, B₂ is any nucleic acid and B₁ is C.
3. mRNA +8G/+11G/+14T/+17C Query

The LOVD3 database was queried for mRNA sequences that contain a nonsense stop codon with +8G/+11G/+14T/+17C positions downstream of the first nucleic acid of the nonsense stop codon. This query returned fifty-eight matching mRNAs from which complementary R-ASOs were generated. See, Table 4

TABLE 4
+8G/+11G/+14T/+17C Database Hits

| | NSC | +8-+27 mRNA sequence | SEQ | R-ASO | SEQ |
|---|---|---|---|---|---|
| Gene | # | 5′-3′ direction | ID | 5′-3′ direction | ID |
| LAMA2 | 1350 | GCAGGATTTCTGAAATCTCA | 185 | TGAGATTTCAGAAATCCTGC | 242 |
| CFTR | 542 | GTGGAATCACACTGAGTGGA | 186 | TCCACTCAGTGTGATTCCAC | 243 |
| PKP2 | 79 | GCAGTGTTCCTGAGTATGTC | 187 | GACATACTCAGGAACACTGC | 244 |
| CRB1 | 362 | GACGCATCACTGGACTGCCT | 188 | AGGCAGTCCAGTGATGCGTC | 245 |
| MYO7A | 1198 | GCTGTTTCGCCCCCTCCGAG | 189 | CTCGGAGGGGGCGAAACAGC | 246 |
| DMD | 3440 | GCAGGCTAGCAGAAATGGAA | 190 | TTCCATTTCTGCTAGCCTGC | 247 |
| CNGA3 | 344 | GGCGCCTCTCCAGGAAGTAC | 191 | GTACTTCCTGGAGAGGCGCC | 248 |
| PKP2 | 79 | GCAGTGTTCCTGAGTATGTC | 192 | GACATACTCAGGAACACTGC | 249 |
| EMD | 44 | GGCGGCTCTCGCCCCCCAGC | 193 | GCTGGGGGGCGAGAGCCGCC | 250 |
| USH2A | 1838 | GAGGAATCCCACAGGAACTG | 194 | CAGTTCCTGTGGGATTCCTC | 251 |
| RDH12 | 194 | GGGGTTTTGCCTATTGCCAC | 195 | GTGGCAATAGGCAAAACCCC | 252 |
| MSH6 | 1163 | GCAGGCTCACACCAATTGAT | 196 | ATCAATTGGTGTGAGCCTGC | 253 |
| EVC | 622 | GCGGCCTCACTGAAGAGTCC | 197 | GGACTCTTCAGTGAGGCCGC | 254 |
| CFTR | 732 | GAAGGCTGTCCTTAGTACCA | 198 | TGGTACTAAGGACAGCCTTC | 255 |
| BRCA1 | 765 | GCAGTATTTCATTGGTACCT | 199 | AGGTACCAATGAAATACTGC | 256 |
| ASH1L | 2421 | GACGGTTGGCAGCTGCAGAG | 200 | CTCTGCAGCTGCCAACCGTC | 257 |
| SMAD4 | 474 | GTGGAATAGCTCCAGCTATC | 201 | GATAGCTGGAGCTATTCCAC | 258 |
| NTRK1 | 166 | GCGGAGTGCCTGAACAGAAG | 202 | CTTCTGTTCAGGCACTCCGC | 259 |
| MEN1 | 108 | GGGGTGTCTCCAGCCGTGAG | 203 | CTCACGGCTGGAGACACCCC | 260 |
| LAMB2 | 1562 | GGAGCCTGGCAGATGTGGAT | 204 | ATCCACATCTGCCAGGCTCC | 261 |
| KIF11 | 53 | GAGGATTGGCTGACAAGAGC | 205 | GCTCTTGTCAGCCAATCCTC | 262 |
| IL12RB1 | 376 | GGGGCCTTGCCACCTGCAGC | 206 | GCTGCAGGTGGCAAGGCCCC | 263 |
| EYS | 1239 | GGAGAATTACCTGTTTAACT | 207 | AGTTAAACAGGTAATTCTCC | 264 |
| COL1A1 | 171 | GAGGAATTTCCGTGCCTGGC | 208 | GCCAGGCACGGAAATTCCTC | 265 |
| ANKRD11 | 838 | GGTGGTTTTCTGACTTGTCC | 209 | GGACAAGTCAGAAAACCACC | 266 |
| WRN | 1371 | GATGTTTTCCCGGTTCTGAA | 210 | TTCAGAACCGGGAAAACATC | 267 |
| UNC80 | 1069 | GCAGAATTTCCCTCCGAAAG | 211 | CTTTCGGAGGGAAATTCTGC | 268 |
| TTI2 | 199 | GGAGACTTTCGGTGATACTA | 212 | TAGTATCACCGAAAGTCTCC | 269 |
| TRIO | 2883 | GAAGCCTCACTGAAGGGAAG | 213 | CTTCCCTTCAGTGAGGCTTC | 270 |
| TCF4 | 567 | GGAGGATGGCCAACAATGCC | 214 | GGCATTGTTGGCCATCCTCC | 271 |
| SETD5 | 272 | GAAGAGTCACTCGTGTTCAA | 215 | TTGAACACGAGTGACTCTTC | 272 |
| SETD1A | 672 | GAGGGATGCCCATGTCCTTC | 216 | GAAGGACATGGGCATCCCTC | 273 |
| RP1L1 | 424 | GGGGACTGGCCCAGCACGTC | 217 | GACGTGCTGGGCCAGTCCCC | 274 |
| PMEL | 516 | GCGGGCTGCCCAAGGAAGCC | 218 | GGCTTCCTTGGGCAGCCCGC | 275 |
| PKD1 | 2528 | GCAGCCTCTCCAGCTACGGA | 219 | TCCGTAGCTGGAGAGGCTGC | 276 |
| PAH | 295 | GCAGCTTTGCCCAGTTTTCC | 220 | GGAAAACTGGGCAAAGCTGC | 277 |
| LTBP4 | 1296 | GCTGCGTCTCCAACGAGAGC | 221 | GCTCTCGTTGGAGACGCAGC | 278 |
| LPL | 88 | GTTGGGTGCCAAAACTTGTG | 222 | CACAAGTTTTGGCACCCAAC | 279 |
| KMT2B | 297 | GTGGGCTCCCCTTTGTGATC | 223 | GATCACAAAGGGGAGCCCAC | 280 |
| ITGB4 | 1759 | GCAGCATCACCACCACCCAC | 224 | GTGGGTGGTGGTGATGCTGC | 281 |
| IKBKAP | 104 | GGAGTGTAGCCAGTGGTATC | 225 | GATACCACTGGCTACACTCC | 282 |
| IGSF1 | 921 | GGAGTGTCTCAGGGAACTCA | 226 | TGAGTTCCCTGAGACACTCC | 283 |
| GK | 419 | GTGGAATTCCACTCAGTCAT | 227 | ATGACTGAGTGGAATTCCAC | 284 |
| GGT1 | 126 | GGGGGCTGTCGGTGGCGGTG | 228 | CACCGCCACCGACAGCCCCC | 285 |
| GFM2 | 117 | GAGGCATTACTATTCAATCA | 229 | TGATTGAATAGTAATGCCTC | 286 |
| F8 | 199 | GGAGTCTGGCCAAGGAAAAG | 230 | CTTTTCCTTGGCCAGACTCC | 287 |
| EYA1 | 185 | GCAGTTTTACAACATCATCA | 231 | TGATGATGTTGTAAAACTGC | 288 |
| EXT2 | 336 | GCTGTGTCCCGGTTGTCATT | 232 | AATGACAACCGGGACACAGC | 289 |
| DYSF | 1734 | GCAGGATCCCAAACCCACAC | 233 | GTGTGGGTTTGGGATCCTGC | 290 |
| DSP | 682 | GCAGGATGACTCTCAAAAAC | 234 | GTTTTTGAGAGTCATCCTGC | 291 |
| COL4A5 | 1342 | GTGGAGTACCTGGATCAGCT | 235 | AGCTGATCCAGGTACTCCAC | 292 |
| CHD7 | 2024 | GTCGAATGCCCGTCAAGCCA | 236 | TGGCTTGACGGGCATTCGAC | 293 |
| CERKL | 362 | GATGGATGTCCCCTAACCAA | 237 | TTGGTTAGGGGACATCCATC | 294 |
| BCOR | 1161 | GACGAGTCTCTAAAGATGAC | 238 | GTCATCTTTAGAGACTCGTC | 295 |
| ATP7A | 974 | GAAGTATCTCCCGAACAGAA | 239 | TTCTGTTCGGGAGATACTTC | 296 |
| APC | 341 | GCTGTATATCCATGCGACAG | 240 | CTGTCGCATGGATATACAGC | 297 |
| AMPD2 | 228 | GTGGGGTGCCGTTCACAGAC | 241 | GTCTGTGAACGGCACCCCAC | 298 |

NSC # = Nonsense stop codon mRNA position.

In one embodiment, the present invention contemplates a nucleic acid antisense oligomer comprising, or consisting of, a plurality of nucleic acids having the sequence of:

5′-C₂₀-C₁₉-C₁₈-C₁₇-C₁₆-C₁₅-C₁₄-C₁₃-C₁₂-C₁₁-C₁₀-C₉-C₈-
C₇-C₆-C₅-C₄-C₃-C₂-C₁-3′

wherein, C₂₀ is any nucleic acid, C₁₉ is any nucleic acid, C₁₈ is any nucleic acid, C₁₇ is any nucleic acid, C₁₆ is any nucleic acid, C₁₅ is any nucleic acid, C₁₄ is any nucleic acid, C₁₃ is any nucleic acid, C₁₂ is any nucleic acid, C₁₁ is any nucleic acid, C₁₀ is G, C₉ is any nucleic acid, C₈ is any nucleic acid, C₇ is A, C₆ is any nucleic acid, C₅ is any nucleic acid, C₄ is C, C₃ is any nucleic acid, C₂ is any nucleic acid and C₁ is C.
4. mRNA +8G/+11G/+14T Query

The LOVD3 database was queried for mRNA sequences that contain a nonsense stop codon with +8G/+11G/+14T positions downstream of the first nucleic acid of the nonsense stop codon. This query returned one hundred and ninety-seven matching mRNAs from which complementary R-ASOs were generated. See, Table 5.

TABLE 5
+8G/+11G/+14T Database Hits

| | NSC | +8-+27 mRNA sequence | SEQ | R-ASO | SEQ |
|---|---|---|---|---|---|
| Gene | # | 5′-3′ direction | ID | 5′-3′ direction | ID |
| DMD | 3190 | GGAGGATCCGTGTCCTGTCT | 299 | AGACAGGACACGGATCCTCC | 496 |
| PKLR | 241 | GCGGCGTCCTGGGCAGCCGG | 300 | CCGGCTGCCCAGGACGCCGC | 497 |
| DMD | 2095 | GGCGATTTGACAGATCTGTT | 301 | AACAGATCTGTCAAATCGCC | 498 |
| CHM | 267 | GACGAGTGGAACAGGTTCCG | 302 | CGGAACCTGTTCCACTCGTC | 499 |
| LAMA2 | 1350 | GCAGGATTTCTGAAATCTCA | 303 | TGAGATTTCAGAAATCCTGC | 500 |
| CFTR | 542 | GTGGAATCACACTGAGTGGA | 304 | TCCACTCAGTGTGATTCCAC | 501 |
| PKP2 | 79 | GCAGTGTTCCTGAGTATGTC | 305 | GACATACTCAGGAACACTGC | 502 |
| CRB1 | 362 | GACGCATCACTGGACTGCCT | 306 | AGGCAGTCCAGTGATGCGTC | 503 |
| MYO7A | 1198 | GCTGTTTCGCCCCCTCCGAG | 307 | CTCGGAGGGGGCGAAACAGC | 504 |
| BRCA2 | 2506 | GCAGTCTGTATCTTGCAAAA | 308 | TTTTGCAAGATACAGACTGC | 505 |
| SLC22A5 | 254 | GGCGGATGCTGCTGGTGGCG | 309 | CGCCACCAGCAGCATCCGCC | 506 |
| SGCE | 97 | GATGGCTTCGATATATCCAA | 310 | TTGGATATATCGAAGCCATC | 507 |
| SMAD4 | 398 | GGTGCCTTAGTGACCACGCG | 311 | CGCGTGGTCACTAAGGCACC | 508 |
| MSH2 | 218 | GAGGAATTCTGATCACAGAA | 312 | TTCTGTGATCAGAATTCCTC | 509 |
| LAMA2 | 2630 | GAGGCATCTTTACAGTTCAA | 313 | TTGAACTGTAAAGATGCCTC | 510 |
| DMD | 3440 | GCAGGCTAGCAGAAATGGAA | 314 | TTCCATTTCTGCTAGCCTGC | 511 |
| CNGA3 | 344 | GGCGCCTCTCCAGGAAGTAC | 315 | GTACTTCCTGGAGAGGCGCC | 512 |
| TSC2 | 1593 | GAGGCCTGGACGTGTGTGGT | 316 | ACCACACACGTCCAGGCCTC | 513 |
| PKP2 | 79 | GCAGTGTTCCTGAGTATGTC | 317 | GACATACTCAGGAACACTGC | 514 |
| MITF | 315 | GAAGATTTAACATAAATGAC | 318 | GTCATTTATGTTAAATCTTC | 515 |
| EMD | 44 | GGCGGCTCTCGCCCCCCAGC | 319 | GCTGGGGGGCGAGAGCCGCC | 516 |
| USH2A | 1838 | GAGGAATCCCACAGGAACTG | 320 | CAGTTCCTGTGGGATTCCTC | 517 |
| TSC2 | 1452 | GTGGCCTCCGGCCCCGAGGT | 321 | ACCTCGGGGCCGGAGGCCAC | 518 |
| RDH12 | 194 | GGGGTTTTGCCTATTGCCAC | 322 | GTGGCAATAGGCAAAACCCC | 519 |
| PRPH2 | 257 | GCAGCCTCATGAACTCCATG | 323 | CATGGAGTTCATGAGGCTGC | 520 |
| NF1 | 1360 | GAAGTGTGTGCCACTGTTTA | 324 | TAAACAGTGGCACACACTTC | 521 |
| MSH6 | 1163 | GCAGGCTCACACCAATTGAT | 325 | ATCAATTGGTGTGAGCCTGC | 522 |
| GCDH | 352 | GCCGCTTGAAGGACCAGGAC | 326 | GTCCTGGTCCTTCAAGCGGC | 523 |
| EXT1 | 701 | GCTGCATGAATACGTTTGCC | 327 | GGCAAACGTATTCATGCAGC | 524 |
| EVC | 622 | GCGGCCTCACTGAAGAGTCC | 328 | GGACTCTTCAGTGAGGCCGC | 525 |
| DYNC2H1 | 3279 | GTCGAGTGTGCCCATTTCTT | 329 | AAGAAATGGGCACACTCGAC | 526 |
| DMD | 992 | GTGGCCTATACTATCTCAGC | 330 | GCTGAGATAGTATAGGCCAC | 527 |
| CFTR | 732 | GAAGGCTGTCCTTAGTACCA | 331 | TGGTACTAAGGACAGCCTTC | 528 |
| BRCA1 | 765 | GCAGTATTTCATTGGTACCT | 332 | AGGTACCAATGAAATACTGC | 529 |
| BRCA1 | 286 | GCAGTTTATTACTCACTAAA | 333 | TTTAGTGAGTAATAAACTGC | 530 |
| ASH1L | 2421 | GACGGTTGGCAGCTGCAGAG | 334 | CTCTGCAGCTGCCAACCGTC | 531 |
| ALDH3A2 | 4 | GGCGGGTCCGACAGGCGTTC | 335 | GAACGCCTGTCGGACCCGCC | 532 |
| USH2A | 1575 | GACGTCTTTATTTTCTTTTT | 336 | AAAAAGAAAATAAAGACGTC | 533 |
| SMAD4 | 474 | GTGGAATAGCTCCAGCTATC | 337 | GATAGCTGGAGCTATTCCAC | 534 |
| SCUBE3 | 929 | GCCGGCTCTATGCCTCTGAA | 338 | TTCAGAGGCATAGAGCCGGC | 535 |
| SBF2 | 1196 | GAGGATTCCATGGGAAGGGA | 339 | TCCCTTCCCATGGAATCCTC | 536 |
| PTCH1 | 604 | GGAGACTGGATATTTTCTGC | 340 | GCAGAAAATATCCAGTCTCC | 537 |
| PKP2 | 393 | GTGGCATCCTCAAGCTTCTG | 341 | CAGAAGCTTGAGGATGCCAC | 538 |
| PHEX | 383 | GATGGCTGGAATTCTCAAGG | 342 | CCTTGAGAATTCCAGCCATC | 539 |
| NTRK1 | 166 | GCGGAGTGCCTGAACAGAAG | 343 | CTTCTGTTCAGGCACTCCGC | 540 |
| NR0B1 | 197 | GCTGCTTTTGCGGTGAAGAC | 344 | GTCTTCACCGCAAAAGCAGC | 541 |
| NEB | 3876 | GAGGCATAGGATGGGTTCCC | 345 | GGGAACCCATCCTATGCCTC | 542 |
| MOCOS | 419 | GCTGCTTCTGTAACACTGGG | 346 | CCCAGTGTTACAGAAGCAGC | 543 |
| MEN1 | 108 | GGGGTGTCTCCAGCCGTGAG | 347 | CTCACGGCTGGAGACACCCC | 544 |
| LTBP2 | 111 | GGCGTGTCCAGCCACCTGCG | 348 | CGCAGGTGGCTGGACACGCC | 545 |
| LAMB2 | 1562 | GGAGCCTGGCAGATGTGGAT | 349 | ATCCACATCTGCCAGGCTCC | 546 |
| KIF11 | 53 | GAGGATTGGCTGACAAGAGC | 350 | GCTCTTGTCAGCCAATCCTC | 547 |
| ISPD | 86 | GTTGGATAAAGGACATTGTT | 351 | AACAATGTCCTTTATCCAAC | 548 |
| IL12RB1 | 376 | GGGGCCTTGCCACCTGCAGC | 352 | GCTGCAGGTGGCAAGGCCCC | 549 |
| IGHMBP2 | 41 | GAGGCGTGTGTTTGCTGAAG | 353 | CTTCAGCAAACACACGCCTC | 550 |
| GBA2 | 745 | GTAGTGTTATGTCTGACCAG | 354 | CTGGTCAGACATAACACTAC | 551 |
| FLNB | 1850 | GTGGTCTGGACTTGGCTATT | 355 | AATAGCCAAGTCCAGACCAC | 552 |
| EYS | 1239 | GGAGAATTACCTGTTTAACT | 356 | AGTTAAACAGGTAATTCTCC | 553 |
| ERCC6 | 947 | GGAGAATAGGCCAGAAGAAG | 357 | CTTCTTCTGGCCTATTCTCC | 554 |
| DMD | 2184 | GAAGCCTGAATCTGCGGTGG | 358 | CCACCGCAGATTCAGGCTTC | 555 |
| DMD | 1965 | GAAGACTCAACTTTGCACAA | 359 | TTGTGCAAAGTTGAGTCTTC | 556 |
| DLG4 | 150 | GCCGCCTCAGGGTCAACGAC | 360 | GTCGTTGACCCTGAGGCGGC | 557 |
| CRB1 | 120 | GAGGTATTTGCCATCAGGAC | 361 | GTCCTGATGGCAAATACCTC | 558 |
| COL4A4 | 945 | GGGGACTGAGAGGGGCCAAA | 362 | TTTGGCCCCTCTCAGTCCCC | 559 |
| COL1A1 | 171 | GAGGAATTTCCGTGCCTGGC | 363 | GCCAGGCACGGAAATTCCTC | 560 |
| BBS2 | 234 | GGAGAATTAAATCGAAAAAT | 364 | ATTTTTCGATTTAATTCTCC | 561 |
| BBS1 | 393 | GTGGCCTGATCATCAAGATC | 365 | GATCTTGATGATCAGGCCAC | 562 |
| ANKRD11 | 838 | GGTGGTTTTCTGACTTGTCC | 366 | GGACAAGTCAGAAAACCACC | 563 |
| ABCA12 | 1249 | GCTGGCTGTGCTGTCTAATC | 367 | GATTAGACAGCACAGCCAGC | 564 |
| WRN | 1371 | GATGTTTTCCCGGTTCTGAA | 368 | TTCAGAACCGGGAAAACATC | 565 |
| WISP3 | 177 | GTAGCCTGGAACCATTACTA | 369 | TAGTAATGGTTCCAGGCTAC | 566 |
| WFS1 | 700 | GCCGCTTCAAGTACGTCCGC | 370 | GCGGACGTACTTGAAGCGGC | 567 |
| USH2A | 1917 | GTGGTCTCCAGCCTTTTACA | 371 | TGTAAAAGGCTGGAGACCAC | 568 |
| UNC80 | 1069 | GCAGAATTTCCCTCCGAAAG | 372 | CTTTCGGAGGGAAATTCTGC | 569 |
| TTN | 35964 | GTGGACTTTATACCCTGAGT | 373 | ACTCAGGGTATAAAGTCCAC | 570 |
| TTI2 | 199 | GGAGACTTTCGGTGATACTA | 374 | TAGTATCACCGAAAGTCTCC | 571 |
| TSC2 | 438 | GCTGGATTCAGAACCTGCAG | 375 | CTGCAGGTTCTGAATCCAGC | 572 |
| TRIO | 2883 | GAAGCCTCACTGAAGGGAAG | 376 | CTTCCCTTCAGTGAGGCTTC | 573 |
| TNFRSF10A | 279 | GGCGCTTGGGTCTCCTACGA | 377 | TCGTAGGAGACCCAAGCGCC | 574 |
| TMPRSS6 | 405 | GGAGGCTGTGTGGCTTGCGC | 378 | GCGCAAGCCACACAGCCTCC | 575 |
| TMPRSS3 | 104 | GCTGTGTCCGGGTGGGTGGT | 379 | ACCACCCACCCGGACACAGC | 576 |
| TCF4 | 567 | GGAGGATGGCCAACAATGCC | 380 | GGCATTGTTGGCCATCCTCC | 577 |
| TAZ | 195 | GGCGCCTGATTGCTGAGTGT | 381 | ACACTCAGCAATCAGGCGCC | 578 |
| SYNGAP1 | 1219 | GGAGGCTGCTGTCCCAGGAA | 382 | TTCCTGGGACAGCAGCCTCC | 579 |
| SYNCRIP | 284 | GAGGCTTTTGCTTTCTTGAA | 383 | TTCAAGAAAGCAAAAGCCTC | 580 |
| STS | 372 | GAGGTATCCGGGTTCCAGGC | 384 | GCCTGGAACCCGGATACCTC | 581 |
| SMAD6 | 14 | GTCGTGTGGTCCCCGACCGG | 385 | CCGGTCGGGGACCACACGAC | 582 |
| SGCB | 28 | GGAGTGTCAATAAAGAGCAC | 386 | GTGCTCTTTATTGACACTCC | 583 |
| SETD5 | 272 | GAAGAGTCACTCGTGTTCAA | 387 | TTGAACACGAGTGACTCTTC | 584 |
| SETD1A | 672 | GAGGGATGCCCATGTCCTTC | 388 | GAAGGACATGGGCATCCCTC | 585 |
| SEMA4A | 521 | GTTGCCTCCTGTCTGCCCCC | 389 | GGGGGCAGACAGGAGGCAAC | 586 |
| RP1L1 | 424 | GGGGACTGGCCCAGCACGTC | 390 | GACGTGCTGGGCCAGTCCCC | 587 |
| RBP4 | 23 | GCAGCTTCCGAGTCAAGGAG | 391 | CTCCTTGACTCGGAAGCTGC | 588 |
| RB1 | 97 | GGGGAATCTGTATCTTTATT | 392 | AATAAAGATACAGATTCCCC | 589 |
| RASA1 | 676 | GCCGATTACAGAAAGGGCAT | 393 | ATGCCCTTTCTGTAATCGGC | 590 |
| RANBP2 | 1785 | GTAGTGTTTGCTGTGTACAA | 394 | TTGTACACAGCAAACACTAC | 591 |
| RAD50 | 1014 | GGTGGCTACAAGATAACCTT | 395 | AAGGTTATCTTGTAGCCACC | 592 |
| RAB3GAP1 | 470 | GAGGGTTGAAAGGAGTGGCA | 396 | TGCCACTCCTTTCAACCCTC | 593 |
| PURA | 163 | GCCGCTTCCTGCGCATCCGC | 397 | GCGGATGCGCAGGAAGCGGC | 594 |
| PTCH1 | 618 | GCAGAGTGATTCAGGTTGAA | 398 | TTCAACCTGAATCACTCTGC | 595 |
| PTCH1 | 184 | GCCGTGTCCATGTATACATG | 399 | CATGTATACATGGACACGGC | 596 |
| PRSS21 | 55 | GGAGCCTGCGCCTGTGGGAT | 400 | ATCCCACAGGCGCAGGCTCC | 597 |
| POT1 | 271 | GGGGAATCAGGGTCTTGCCA | 401 | TGGCAAGACCCTGATTCCCC | 598 |
| POT1 | 140 | GGGGAATCAGGGTCTTGCCA | 402 | TGGCAAGACCCTGATTCCCC | 599 |
| POGZ | 1006 | GACGTTTCCAGGCCTCCCAG | 403 | CTGGGAGGCCTGGAAACGTC | 600 |
| PMEL | 516 | GCGGGCTGCCCAAGGAAGCC | 404 | GGCTTCCTTGGGCAGCCCGC | 601 |
| PKP2 | 727 | GTGGCCTGCAGCACACCCGA | 405 | TCGGGTGTGCTGCAGGCCAC | 602 |
| PKLR | 508 | GAGGAGTCTTCCCCTTGCTT | 406 | AAGCAAGGGGAAGACTCCTC | 603 |
| PKD2 | 48 | GGGGCCTGGAGATCGAGATG | 407 | CATCTCGATCTCCAGGCCCC | 604 |
| PKD1 | 3114 | GCCGCTTCAAGTACGAGATC | 408 | GATCTCGTACTTGAAGCGGC | 605 |
| PKD1 | 2528 | GCAGCCTCTCCAGCTACGGA | 409 | TCCGTAGCTGGAGAGGCTGC | 606 |
| PKD1 | 227 | GGTGCCTGTGTGGGGCGGCC | 410 | GGCCGCCCCACACAGGCACC | 607 |
| PAX3 | 40 | GCGGTGTTTTTATCAACGGC | 411 | GCCGTTGATAAAAACACCGC | 608 |
| PALB2 | 650 | GCTGTATTTTTCCAGAGGAA | 412 | TTCCTCTGGAAAAATACAGC | 609 |
| PAH | 295 | GCAGCTTTGCCCAGTTTTCC | 413 | GGAAAACTGGGCAAAGCTGC | 610 |
| OPA1 | 902 | GGCGATTAGAGAAAAATGTT | 414 | AACATTTTTCTCTAATCGCC | 611 |
| NR2E3 | 332 | GGGGCCTGAAGGATCCTGAG | 415 | CTCAGGATCCTTCAGGCCCC | 612 |
| NR0B1 | 130 | GCTGCTTTTGTGGTGAAGAC | 416 | GTCTTCACCACAAAAGCAGC | 613 |
| NLRP1 | 132 | GAAGGGTTTTGAGACAGCTG | 417 | CAGCTGTCTCAAAACCCTTC | 614 |
| NEB | 5819 | GTGGAATTGGGTGGATGCCA | 418 | TGGCATCCACCCAATTCCAC | 615 |
| NDP | 110 | GGGGCATGCGACTCACTGCC | 419 | GGCAGTGAGTCGCATGCCCC | 616 |
| MNX1 | 290 | GGCGGATGAAATGGAAACGC | 420 | GCGTTTCCATTTCATCCGCC | 617 |
| MLH1 | 52 | GAGGCCTGAAGTTGATTCAG | 421 | CTGAATCAACTTCAGGCCTC | 618 |
| MITF | 214 | GAAGATTTAACATAAATGAC | 422 | GTCATTTATGTTAAATCTTC | 619 |
| MBD5 | 25 | GTCGTGTGGATCAAAATGGA | 423 | TCCATTTTGATCCACACGAC | 620 |
| LTBP4 | 1377 | GTGGCTTTGGACTCCCCTAC | 424 | GTAGGGGAGTCCAAAGCCAC | 621 |
| LTBP4 | 1296 | GCTGCGTCTCCAACGAGAGC | 425 | GCTCTCGTTGGAGACGCAGC | 622 |
| LPL | 88 | GTTGGGTGCCAAAACTTGTG | 426 | CACAAGTTTTGGCACCCAAC | 623 |
| LDLR | 811 | GGCGGCTTAAGAACATCAAC | 427 | GTTGATGTTCTTAAGCCGCC | 624 |
| LDLR | 75 | GCCGTGTCAACCGCTGCATT | 428 | AATGCAGCGGTTGACACGGC | 625 |
| LCA5 | 572 | GTAGTTTTTTGGATTTCCAA | 429 | TTGGAAATCCAAAAAACTAC | 626 |
| KMT2B | 297 | GTGGGCTCCCCTTTGTGATC | 430 | GATCACAAAGGGGAGCCCAC | 627 |
| KIAA2022 | 146 | GCTGCTTCATGGAATCCAAG | 431 | CTTGGATTCCATGAAGCAGC | 628 |
| KDM5C | 424 | GGAGGCTGGTAAATAGCATT | 432 | AATGCTATTTACCAGCCTCC | 629 |
| KCNV2 | 177 | GCCGCTTCCTGGAGGAGCTG | 433 | CAGCTCCTCCAGGAAGCGGC | 630 |
| KCNJ10 | 26 | GGAGAGTCCTGACAAAAGAT | 434 | ATCTTTTGTCAGGACTCTCC | 631 |
| KAT6A | 1019 | GGAGAGTCCGAAAGCGCAAA | 435 | TTTGCGCTTTCGGACTCTCC | 632 |
| ITGB4 | 1759 | GCAGCATCACCACCACCCAC | 436 | GTGGGTGGTGGTGATGCTGC | 633 |
| ITGB2 | 734 | GGCGCTTTGAGAAGGAGAAG | 437 | CTTCTCCTTCTCAAAGCGCC | 634 |
| IMPG2 | 212 | GCAGCTTGGAAAGGCCAGAG | 438 | CTCTGGCCTTTCCAAGCTGC | 635 |
| IKBKAP | 104 | GGAGTGTAGCCAGTGGTATC | 439 | GATACCACTGGCTACACTCC | 636 |
| IGSF1 | 921 | GGAGTGTCTCAGGGAACTCA | 440 | TGAGTTCCCTGAGACACTCC | 637 |
| IDUA | 500 | GGCGCATGCGCGCGGCTGAG | 441 | CTCAGCCGCGCGCATGCGCC | 638 |
| IDUA | 486 | GGCGCCTGGGCCGGCCCGTC | 442 | GACGGGCCGGCCCAGGCGCC | 639 |
| HSD11B2 | 261 | GCTGCTTCAAGACAGAGTCA | 443 | TGACTCTGTCTTGAAGCAGC | 640 |
| HPS6 | 412 | GGAGCCTGCGGGGTGCCCAG | 444 | CTGGGCACCCCGCAGGCTCC | 641 |
| GPR179 | 237 | GACGGCTCCGACCTGGATGG | 445 | CCATCCAGGTCGGAGCCGTC | 642 |
| GMPPB | 264 | GCAGCATTGGCCCCAATGTG | 446 | CACATTGGGGCCAATGCTGC | 643 |
| GLA | 102 | GCAGACTTCAGGCAGACCCT | 447 | AGGGTCTGCCTGAAGTCTGC | 644 |
| GK | 419 | GTGGAATTCCACTCAGTCAT | 448 | ATGACTGAGTGGAATTCCAC | 645 |
| GGT1 | 126 | GGGGGCTGTCGGTGGCGGTG | 449 | CACCGCCACCGACAGCCCCC | 646 |
| GFM2 | 117 | GAGGCATTACTATTCAATCA | 450 | TGATTGAATAGTAATGCCTC | 647 |
| GDF1 | 209 | GCAGCCTCCGCCTGGCGCTG | 451 | CAGCGCCAGGCGGAGGCTGC | 648 |
| FTHL17 | 94 | GCGGGCTCGTGGCCATGGAG | 452 | CTCCATGGCCACGAGCCCGC | 649 |
| F8 | 199 | GGAGTCTGGCCAAGGAAAAG | 453 | CTTTTCCTTGGCCAGACTCC | 650 |
| F8 | 1124 | GGTGGATACAAAGGACTCAT | 454 | ATGAGTCCTTTGTATCCACC | 651 |
| EYA1 | 185 | GCAGTTTTACAACATCATCA | 455 | TGATGATGTTGTAAAACTGC | 652 |
| EXT2 | 336 | GCTGTGTCCCGGTTGTCATT | 456 | AATGACAACCGGGACACAGC | 653 |
| ELN | 150 | GTGGCGTGCTCCCAGGAGCT | 457 | AGCTCCTGGGAGCACGCCAC | 654 |
| DYSF | 1734 | GCAGGATCCCAAACCCACAC | 458 | GTGTGGGTTTGGGATCCTGC | 655 |
| DSP | 682 | GCAGGATGACTCTCAAAAAC | 459 | GTTTTTGAGAGTCATCCTGC | 656 |
| DSC3 | 710 | GTGGAGTTTTTGGTGCAACT | 460 | AGTTGCACCAAAAACTCCAC | 657 |
| DMD | 548 | GCTGGGTTCTTTTACAAGAC | 461 | GTCTTGTAAAAGAACCCAGC | 658 |
| D2HGDH | 169 | GCTGCGTCCTGGAGGAGCTG | 462 | CAGCTCCTCCAGGACGCAGC | 659 |
| CUL3 | 436 | GGAGACTTCTCACAAATAAA | 463 | TTTATTTGTGAGAAGTCTCC | 660 |
| COL4A5 | 1342 | GTGGAGTACCTGGATCAGCT | 464 | AGCTGATCCAGGTACTCCAC | 661 |
| CNOT3 | 694 | GGCGATTCCACACCAAGTAC | 465 | GTACTTGGTGTGGAATCGCC | 662 |
| CNKSR2 | 849 | GCGGGTTCAACCATTGCTGT | 466 | ACAGCAATGGTTGAACCCGC | 663 |
| CNGB3 | 231 | GCTGTTTTATACCACTGCGC | 467 | GCGCAGTGGTATAAAACAGC | 664 |
| C1B1 | 72 | GCAGGGTCTTCTCCACATCC | 468 | GGATGTGGAGAAGACCCTGC | 665 |
| CHD7 | 2024 | GTCGAATGCCCGTCAAGCCA | 469 | TGGCTTGACGGGCATTCGAC | 666 |
| CHD3 | 457 | GCCGCGTATGCAAGGACGGC | 470 | GCCGTCCTTGCATACGCGGC | 667 |
| CHD3 | 1137 | GGGGCCTGGGCATCAATCTG | 471 | CAGATTGATGCCCAGGCCCC | 668 |
| CERKL | 362 | GATGGATGTCCCCTAACCAA | 472 | TTGGTTAGGGGACATCCATC | 669 |
| CELF2 | 83 | GTTGTTTCGTAACATTTTAT | 473 | ATAAAATGTTACGAAACAAC | 670 |
| CELF2 | 59 | GTTGTTTCGTAACATTTTAT | 474 | ATAAAATGTTACGAAACAAC | 671 |
| CD3D | 93 | GCTGTGTGGAGCTGGATCCA | 475 | TGGATCCAGCTCCACACAGC | 672 |
| CACNA1F | 495 | GCTGCCTAAACAAGATCATG | 476 | CATGATCTTGTTTAGGCAGC | 673 |
| BRWD3 | 729 | GAAGAGTGGTGGTCAATGAA | 477 | TTCATTGACCACCACTCTTC | 674 |
| BEST1 | 24 | GCAGCATCTACAAGCTGCTA | 478 | TAGCAGCTTGTAGATGCTGC | 675 |
| BCOR | 1161 | GACGAGTCTCTAAAGATGAC | 479 | GTCATCTTTAGAGACTCGTC | 676 |
| ATP7A | 974 | GAAGTATCTCCCGAACAGAA | 480 | TTCTGTTCGGGAGATACTTC | 677 |
| ATM | 2762 | GTGGTGTTCTTGAATGGTGC | 481 | GCACCATTCAAGAACACCAC | 678 |
| ARX | 84 | GGCGCCTGTACGGCCCGGGC | 482 | GCCCGGGCCGTACAGGCGCC | 679 |
| ARMC4 | 947 | GCTGTATGTGGGGCAGGAAT | 483 | ATTCCTGCCCCACATACAGC | 680 |
| APC | 874 | GAGGTTTGCAGATCTCCACC | 484 | GGTGGAGATCTGCAAACCTC | 681 |
| APC | 848 | GAAGTTTGGAGAGAGAACGC | 485 | GCGTTCTCTCTCCAAACTTC | 682 |
| APC | 837 | GAAGCTTAGATAGTTCTCGT | 486 | ACGAGAACTATCTAAGCTTC | 683 |
| APC | 468 | GGGGACTACAGGCCATTGCA | 487 | TGCAATGGCCTGTAGTCCCC | 684 |
| APC | 341 | GCTGTATATCCATGCGACAG | 488 | CTGTCGCATGGATATACAGC | 685 |
| ANK1 | 1252 | GGCGCCTGCGCTGCTACTGC | 489 | GCAGTAGCAGCGCAGGCGCC | 686 |
| AMPD2 | 228 | GTGGGGTGCCGTTCACAGAC | 490 | GTCTGTGAACGGCACCCCAC | 687 |
| ALPI | 118 | GCGGGGTCAAGGCCAACTTC | 491 | GAAGTTGGCCTTGACCCCGC | 688 |
| ALDH7A1 | 476 | GTGGCATTGTAAATGTCAAC | 492 | GTTGACATTTACAATGCCAC | 689 |
| AHI1 | 1089 | GCAGCATAGGAAAGGGACAG | 493 | CTGTCCCTTTCCTATGCTGC | 690 |
| ADAMTS18 | 33 | GCTGCCTCTGCTGTGCGTCG | 494 | CGACGCACAGCAGAGGCAGC | 691 |
| ABCA4 | 2104 | GCCGCATGCTGTGGAACGTC | 495 | GACGTTCCACAGCATGCGGC | 692 |

NSC # = Nonsense stop codon mRNA position.

In one embodiment, the present invention contemplates a nucleic acid antisense oligomer comprising, or consisting of, a plurality of nucleic acids having the sequence of:

5′-D₂₀-D₁₉-D₁₈-D₁₇-D₁₆-D₁₅-D₁₄-D₁₃-D₁₂-D₁₁-D₁₀-D₉-D₈-
D₇-D₆-D₅-D₄-D₃-D₂-D₁-3′

wherein, D₂₀ is any nucleic acid, D₁₉ is any nucleic acid, D₁₈ is any nucleic acid, D₁₇ is any nucleic acid, D₁₆ is any nucleic acid, D₁₅ is any nucleic acid, D₁₄ is any nucleic acid, D₃ is any nucleic acid, D₁₂ is any nucleic acid, D₁₁ is any nucleic acid, D₁₀ is any nucleic acid, D₉ is any nucleic acid, D₈ is any nucleic acid, D₇ is A, D₆ is any nucleic acid, D₅ is any nucleic acid, D₄ is C, D₃ is any nucleic acid, D₂ is any nucleic acid and D₁ is C.

### VIII. Clinical Applications

In one embodiment, the present invention contemplates a method for treating a genetic disease caused by an mRNA with a nonsense stop codon and a guanosine at the eighth position from the first nucleic acid of the nonsense stop codon with a composition comprising a nucleic acid antisense oligomer and a suppressor tRNA. Genetic diseases have been reported caused by nonsense mutations which are more frequent than the CFTR nonsense G542X stop codon and reflect a gene context amenable to suppression by the nucleic acid antisense oligomers disclosed herein. See, Table 6; LOVD3 database.

TABLE 6
Exemplary genetic diseases caused by mRNA's with nonsense
stop codons resulting in truncated protein expression.

| Genetic Disease | Gene | Encoded Protein |
|---|---|---|
| Duchenne muscular dystrophy | DMD | Dystrophin |
| Nonspherocytic hemolytic anemia | PKLR | Pyruvate kinase |
| Inherited retinal diseases (IRD) | PRPH2 | Peripherin 2 |
| Ataxia-telangiectasia | ATM | Serine/threonine kinase |
| Miyoshi myopathy, limb-girdle | DYSF | Dysferlin |
| muscular dystrophy | | |
| type 2B, and distal | | |
| anterior compartment myopathy. | | |
| Breast and ovarian cancer | BRCA1 | Breast cancer 1 |
| | BRCA2 | Breast cancer 2 |
| Stickler syndrome | COL2A1 | Pro-alpha1(II) |
| Choroideremia | CHM | Rab escort protein |
| Bull's-eye maculopathy | ABCA4 | ATP binding cassette |
| | | subfamily A |
| | | member 4 |
| Familial breast and | PALB2 | Partner and localizer |
| pancreatic cancer | | of BRCA2 |
| Neurofibromatosis type 1 | NF1 | Neurofibromin |
| Usher Syndrome | MYO7A | Myosin VIIA |
| LAMA2-related | LAMA2 | Laminin 2 |
| muscular dystrophy | | alpha-2 subunit |
| Cystic fibrosis | CFTR | Cystic fibrosis |
| | | transmembrane |
| | | conductance regulator |
| Rett Syndrome | MECP2 | Methyl CpG binding |
| | | protein 2 |
| Recessive retinitis pigmentosa | EYS | Eyes shut homolog |
| Tuberous sclerosis complex 1 | TSC1 | Hamartin |
| Recessive juvenile | TULP1 | Tubby-like protein 1 |
| retinitis pigmentosa | | |
| Tuberous sclerosis complex 2 | TSC2 | Tuberin |
| Progressive pseudorheumatoid | WISP3 | Wingless/integrated 1 |
| dysplasia | | |
| Epilepsy | GRN | Progranulin |
| Hemophilia | F8 | Coagulation factor VIII |

TABLE 7
Representative Therapeutic R-ASOs
And Complementary mRNA Regions

| | Stop | | SEQ | | SEQ |
|---|---|---|---|---|---|
| Gene | Codon | +8-+27 mRNA (5′→3′) | # | R-ASO (5′→3′) | # |
| ABCA4 | 152 | GGGATATCTTGAAAGATGAA | 693 | TTCATCTTTCAAGATATCCC | 712 |
| BRCA1 | 1835 | GGGTGTTGGACAGTGTAGCA | 694 | TGCTACACTGTCCAACACCC | 713 |
| BRCA1 | 1751 | GAGAATCCCAGGACAGAAAG | 695 | CTTTCTGTCCTGGGATTCTC | 714 |
| BRCA1 | 780 | GTATCTCGTTACTGGAAGTT | 696 | AACTTCCAGTAACGAGATAC | 715 |
| EYS | 2935 | GCCTGTGTACTTTGGGTTGG | 697 | CCAACCCAAAGTACACAGGC | 716 |
| BRCA1 | 1203 | GGGCCAAGAAATTAGAGTCC | 698 | GGACTCTAATTTCTTGGCCC | 717 |
| TSC1 | 509 | GTCTCCCAGGTTCTCAGCGG | 699 | CCGCTGAGAACCTGGGAGAC | 718 |
| TULP1 | 301 | GCACGGTGCGCTGCCGGCTG | 700 | CAGCCGGCAGCGCACCGTGC | 719 |
| DMD | 3190 | GGAGGATCCGTGTCCTGTCT | 701 | AGACAGGACACGGATCCTCC | 720 |
| DMD | 3391 | GCTACCTGCCAGTGCAGACT | 702 | AGTCTGCACTGGCAGGTAGC | 721 |
| TSC2 | 1138 | GCGCCCTGGACGTGCCGGCC | 703 | GGCCGGCACGTCCAGGGCGC | 722 |
| CLCN | 1894 | GTACCGGGGCACCTCCATCT | 704 | AGATGGAGGTGCCCCGGTAC | 723 |
| PKLR | 241 | GCGGCGTCCTGGGCAGCCGG | 705 | CCGGCTGCCCAGGACGCCGC | 724 |
| WISP3 | 52 | GCCCTCAGCAGAAGCCCCGT | 706 | ACGGGGCTTCTGCTGAGGGC | 725 |
| NF1 | 1947 | GAGTTACTGCTATTCTTGAC | 707 | GTCAAGAATAGCAGTAACTC | 726 |
| TSC1 | 245 | GGAAGAGATTAGAAACTCAT | 708 | ATGAGTTTCTAATCTCTTCC | 727 |
| GRN | 493 | GCGAGAAGGAAGTGGTCTCT | 709 | AGAGACCACTTCCTTCTCGC | 728 |
| TSC1 | 500 | GCTTTGACTCTCCCTTTTAC | 710 | GTAAAAGGGAGAGTCAAAGC | 729 |
| F8 | 814 | GTCCTACTCCACATGGGCTA | 711 | TAGCCCATGTGGAGTAGGAC | 730 |

Other genes encoding mRNAs with nonsense codons that result in genetic diseases that are also known include, but are not limited to, CRB1, SLC22A5, SGCE, SMAD4, MSH2, CNGA3, MITF, EMD, USH2A, RDH12, MSH6, GCDH, EXT1, EVC, DYNC2H1, ASHIL, ALDH3A2, USH2A, SCUBE3, SBF2, PTCH1, PHEX, NTRK1, NROB1, NEB, MOCOS, MENI, LTBP2, LAMB2, KIF 11, ISPD, IL12RB1, IGHTMBP2, GBA2, FLNB, ERCC6, DLG4, CRB1, COL4A4, COL1A1, BBS2, BBS1, ANKRD11, ABCA12, WRN, WFS1, USH2A, UNC80, TTN, TT12, TRIO, TNFRSF10A, TMPRSS6, TMIPRSS3, TCF4, TAZ, SYNGAP1, SYNCRIP, STS, SMAD6, SGCB, SETDS, SETIA, SEMA4A, RP1L1, RBP4, RB1, RASA1, RANBP2, RAD50, RAB3GAP1, PURA, PTCH1, PRSS21, POT1, POGZ, PMEL, PKD2, PKD1, PAX3, PAH, OPA1, NR2E3, NROB1, NLRP1, NEB, NDP, MNX1, MLH1, MBD5, LTBP4, LPL, LDLR, LCAS, KMT2B, KIAA2022, KDM5C, KCNV2, KCNJ10, KAT6A, ITGB4, ITGB2, IMPG2, IKBKAP, IGSF1, IDUA, HSD11B2, HPS6, GPR179, GMPPB, GLA, GK, GGT1, GFM2, GDF1, FTHL17, EYA1, EXT2, ELN, DSP, DSC3, D2HGDH, CUL3, COL4A5, CNOT3, CNKSR2, CNGB3, CIBI, CHD7, CHD3, CERKL, CELF2, CD3D, CACNA1F, BRWD3, BEST1, BCOR, ATP7A, ARX, ARMC4, APC, ANK1, AMPD2, ALPI, ALDH7A1, AHIl, ADAMTS18 and ABCA4. See, medlineplus.gov genetics gene.

### A. Cystic Fibrosis

In one embodiment, the present invention contemplates a plurality of specific nucleic acid +4-+9 (e.g., +4, +5, +6, +7, +8, +9) antisense oligomers that are useful in the treatment of cystic fibrosis. Combining these antisense oligomers with suppressor tRNAs results in synergistic clinically relevant results.

Cystic fibrosis is an inherited life-threatening disorder or disease that damages the lungs and digestive system. In particular, cystic fibrosis affects the cells that produce mucus, sweat, and digestive juices and causes these fluids to become thick and sticky. The causative factor of cystic fibrosis is believed to be a genetic mutation G542X having a population frequency of ~2.4%,

In one embodiment, the present invention contemplates a modified cystic fibrosis transmembrane conductance regulator (CFTR) deoxyribonucleic acid (DNA) construct. In one embodiment, the modified CFTR DNA construct comprises a CFTR open reading frame comprising a premature TGA stop codon. In one embodiment, the TGA premature stop codon is a G542X mutation. In one embodiment, the modified CFTR DNA construct further comprises a nanoluciferase gene and a natural stop codon. In one embodiment, the modified CFTR mRNA construct molecule is hybridized to a single stranded nucleic acid antisense oligomer at the +8 nucleotide position downstream of a TGA premature stop codon. In one embodiment, the nucleic acid antisense oligomer is 5′-CTCGTTGACCTCCACTCAGTGTGATTCCAC-3′. See, FIG. 6.

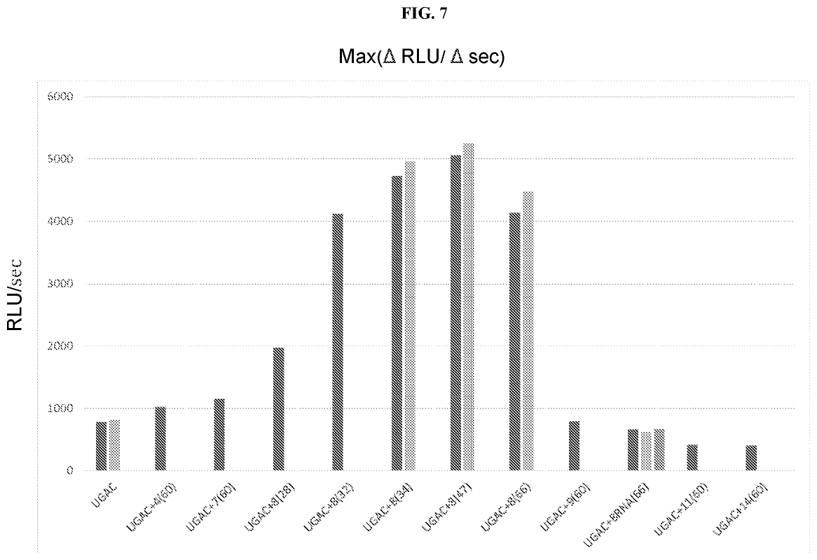

A 511-565 CFTR mRNA was designed with several different codon configurations: i) a single wild type sense codon (“GGAG”) and a subsequent wild-type stop codon; ii) a premature stop codon “TGAG” comprising a G542X mutation (e.g., a strong stop codon); and iii) a premature stop codon “TGAC” comprising a G542X mutation (a weak stop codon) rendering an artificial readthrough-prone context. The data showed that the CFTR mRNA with the “TGAC” premature stop codon provided a nucleic acid antisense oligomer hybridizing at the +8 nucleotide position downstream of the “TGAC” premature stop codon and showed significant translation readthrough as compared to +4, +7, +9, +11 and +14 nucleic acid downstream positions. Surprisingly, the +8(32), +8(34), +8(47) and +8(66) nucleic acid antisense oligomers showed between 4-5 fold higher readthrough as compared to the other downstream positions. Nevertheless, nucleic acid antisense oligomers that hybridized to positions +4 and +7 showed progressively higher readthrough than background readthrough, whereas nucleic acid antisense oligomers that hybridized at positions +9, +11 and +14 showed reduced readthrough. See, FIG. 7.

The CFTR premature stop codon DNA construct expression readthrough analysis was also performed with the aminoglycoside G418, having the structure of:

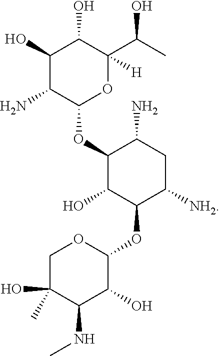 [SMILES: CN[C@@H]1[C@@H](O)[C@@H](O[C@H]2[C@H](O)[C@@H](O[C@H]3O[C@H]([C@H](C)O)[C@@H](O)[C@H](O)[C@H]3N)[C@H](N)C[C@@H]2N)OC[C@]1(C)O]

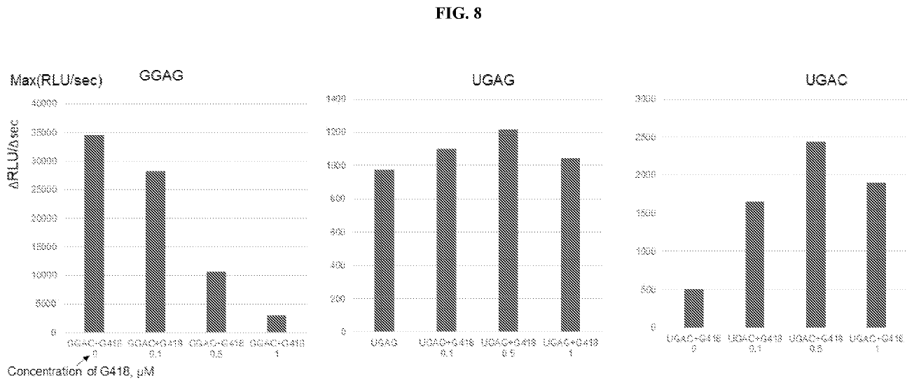

The data showed that G418 inhibits wild type sense codon (GGAG) readthrough in a concentration-dependent manner. Additionally, G418 promotes premature stop codon (UGAG and UGAC) readthrough in a concentration-dependent manner. See, FIG. 8.

Although these molecules result in stop-codon readthrough on the mutant mRNA, broad miscoding of cellular mRNAs makes such molecules toxic and generally poor therapeutic agents (Keeling et al. 2014; Dabrowski et al. 2018).

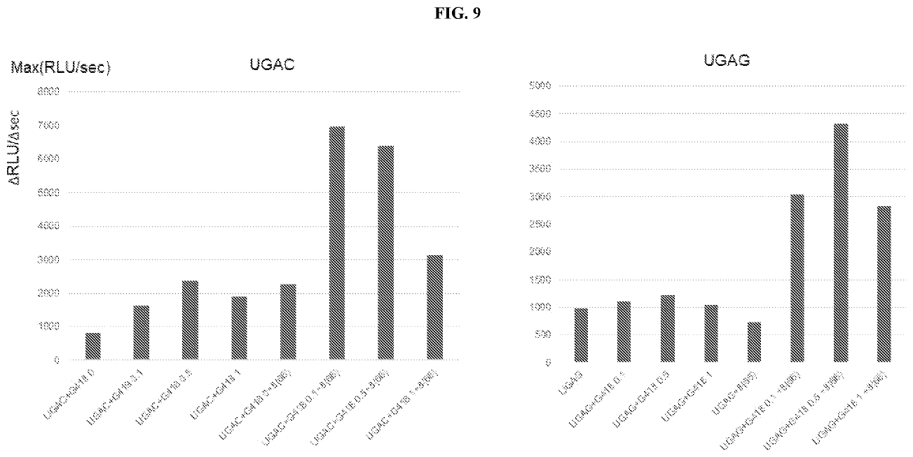

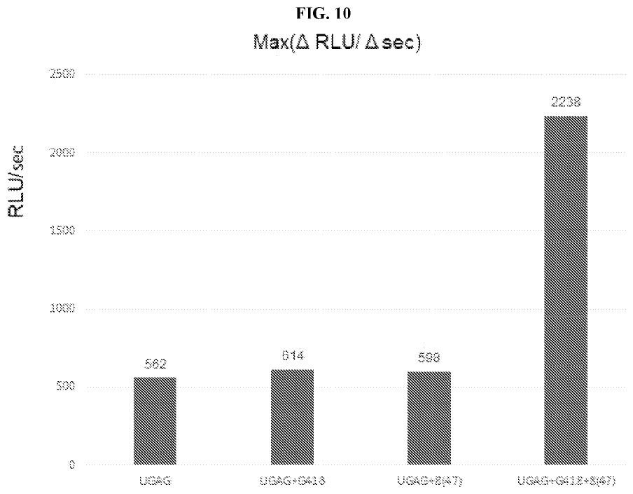

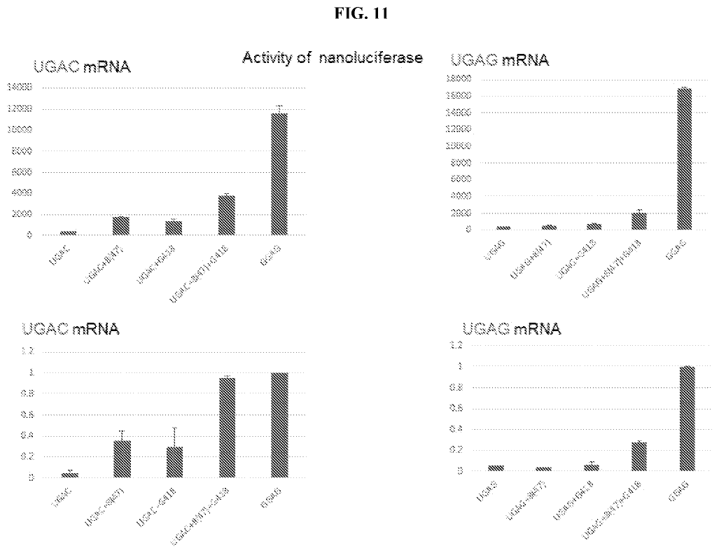

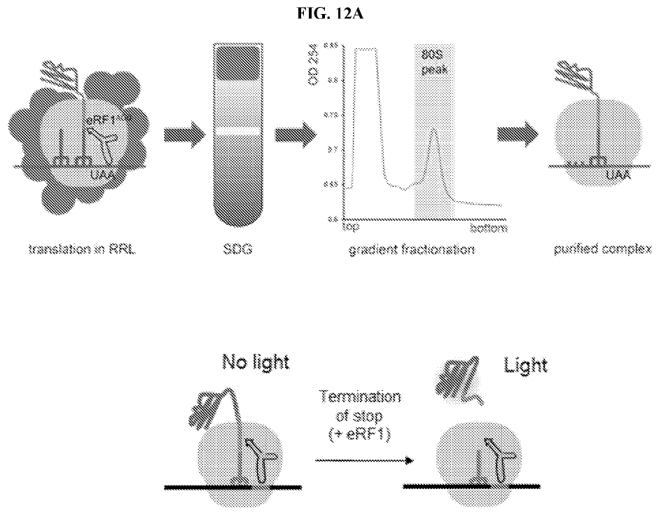

Because aminoglycosides are used in the clinical therapy of some premature stop codon-associated diseases, nucleic acid antisense oligomers and G418 were evaluated for a synergistic effect. The data show that a combination of a +8(66) antisense oligomer and G418 do provide a synergistic effect of up to 5-fold promoting readthrough of premature stop codon using a CFTR DNA expression construct, when compared to either G418 or a +8(66) nucleic acid antisense oligomer alone. See, FIG. 9. A strong UGAG (patient mutation context) premature stop codon is harder to readthrough than a UGAC premature stop codon. Surprisingly, 0.5 μM and 0.1 μM G418 concentrations had a significantly higher readthrough rate than the 1 μM G418 concentration of both the weak (UGAC) and strong UGAG premature codon (patient mutation context). Similar synergistic data was observed when using G418 and a +8(47) nucleic acid antisense oligomer in combination as opposed to alone with the CFTR UGAG mRNA. See, FIG. 10.

mRNA readthrough induced by the presently disclosed nucleic acid antisense oligomers was validated by correlating nanoluciferase activity with expressed protein level. See, FIG. 11. These data show that translation readthrough as revealed by increased luciferase signal correlates with a proportional increased amount of full-length protein.

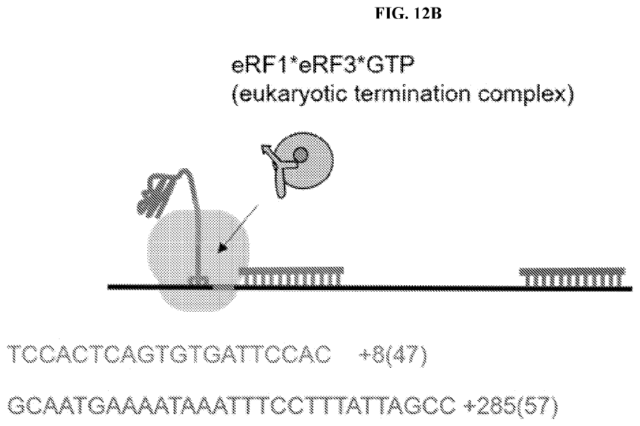

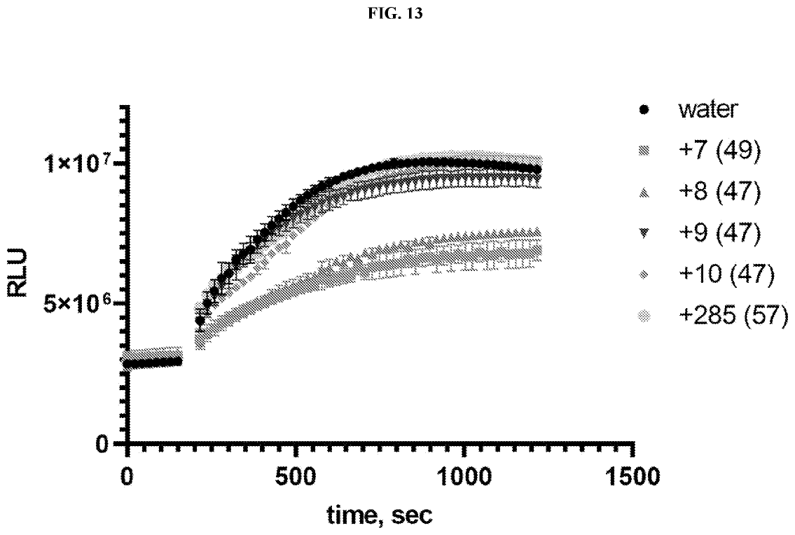

The termination step during a nucleic acid antisense oligomer-induced translation readthrough of a stop codon was assessed with a modified luciferase assay (e.g., Termiluc). In brief, this assay identifies translation termination upon the appearance of light in the presence of an eRF1 eukaryotic termination protein. See, FIG. 12A and FIG. 12B. The data show that readthrough-promoting nucleic acid antisense oligomers inhibit termination translation in vitro in a sequence-specific manner. See, FIG. 13.

Termination of translation was accompanied by an inhibited protein release as shown with nucleic acid antisense oligos +7 and +8 using a Termiluc assay. See, FIG. 13. These data are consistent with readthrough data obtained in a rabbit reticulocyte lysate (RRL) assay. Additionally, the +9 antisense oligo was least efficient, also consistent with the RRL data.

These data suggest that nucleic acid antisense oligomers as contemplated herein specifically interfere with the translation termination step, thereby lowering the release of truncated protein and allowing the ribosome to continue translation. Although it is not necessary to understand the mechanism of an invention, it is believed that the presently disclosed stop codon readthrough technology is programmable to provide specificity for a particular stop codon of a particular mRNA.

### B. Rett Syndrome

The data presented herein demonstrate that specific nucleic acid +4-+9 (e.g., +4, +5, +6, +7, +8, +9) antisense oligomers are useful in the treatment of Rett syndrome. Combining these antisense oligomers with suppressor tRNAs results in synergistic clinically relevant results.

Rett syndrome is a rare genetic mutation affecting brain development that has primarily been found in females. Briefly, infants seem healthy during their first six months, but over time, rapidly lose coordination, speech, and use of the hands. Symptoms may then stabilize for years. There's no cure, but medications, physical and speech therapy, and nutritional support help manage symptoms, prevent complications, and improve quality of life. Recently, a genetic basis has been found that appears to involve premature stop codons.

It is believed that a dysfunctional brain protein, methylcytosine-binding protein 2 (Mecp2), may be responsible for many of the Rett syndrome symptoms. The MECP2 gene provides instructions for making a protein called MeCP2. This protein helps regulate gene activity (expression) by modifying chromatin, the complex of DNA and protein that packages DNA into chromosomes. The MeCP2 protein is present in cells throughout the body, although it is particularly abundant in brain cells.

In the brain, the MeCP2 protein is important for the function of several types of cells, including nerve cells (neurons). The protein likely plays a role in maintaining connections (synapses) between neurons, where cell-to-cell communication occurs. Many of the genes that are known to be regulated by the MeCP2 protein play a role in normal brain function, particularly the maintenance of synapses.

Those skilled in the art believe that the MeCP2 protein may also be involved in processing molecules called messenger RNA (mRNA), which serve as genetic blueprints for making proteins. By cutting and rearranging mRNA molecules in different ways, the MeCP2 protein controls the production of different versions of certain proteins. This process is known as alternative splicing. In the brain, the alternative splicing of proteins plays a role in normal communication between neurons and may also be necessary for the function of other types of brain cells.

Most cases of Rett syndrome are caused by a mutation in methylcytosine-binding protein 2 (MECP2) gene. The MECP2 gene is located on the X chromosome. Between 90% and 95% of girls with Rett syndrome have a mutation in the MECP2 gene. Amir et al., “Rett syndrome is caused by mutations in MECP2” Nature Genetics 23(2):185-188 (1999); Schollen et al., “Gross rearrangements in the MECP2 gene in three patients with Rett syndrome: Implications for routine diagnosis of Rett syndrome” Human Mutations 22:116-120 (2003); and Zoghbi, H. Y. “MeCP2 dysfunction in humans and mice” Journal of Child Neurology 20:736-740 (2005). It is generally believed that about eight (8) mutations in the MECP2 gene are responsible for the most prevalent causes of Rett syndrome. The development and severity of Rett syndrome symptoms depend on the location and type of the mutation on the MECP2 gene. Percy et al., “Rett syndrome: North American database” Journal of Child Neurology 22(12):1338-1341 (2007).

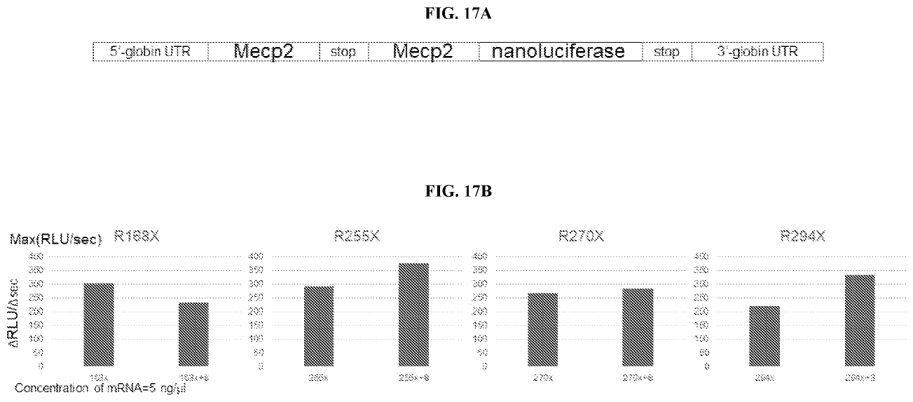

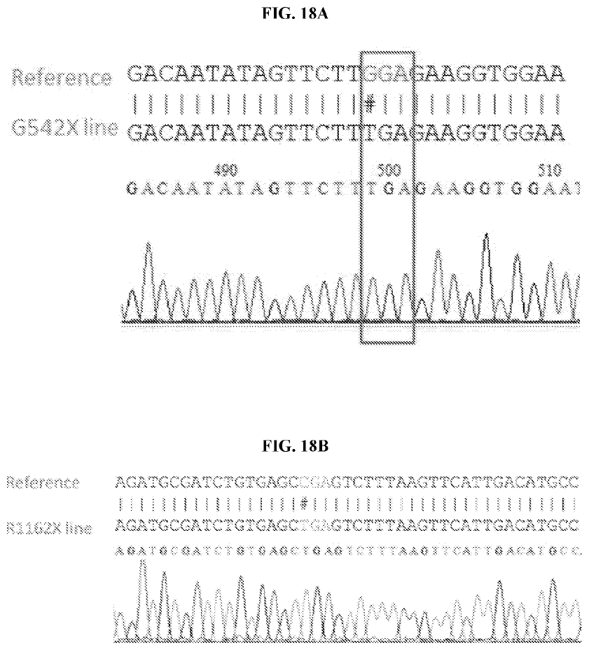

The data presented herein use an Mecp2 mRNA with one of four (4) premature stop codons comprising nonsense mutations (e.g., R168X, R255X, R270X or R294X). See, FIG. 17A. The data show that a +8 nucleic acid antisense oligomer, only mildly enhanced readthrough of mRNAs at three out of four premature stop codons. See, FIG. 17B.

### C. Choroideremia

The data presented herein demonstrate that specific nucleic acid +4-+9 (e.g., +4, +5, +6, +7, +8, +9) antisense oligomers are useful in the treatment of choroideremia. Combining these antisense oligomers with suppressor tRNAs results in synergistic clinically relevant results.

Choroideremia (CHM) is a rare, recessive form of hereditary retinal degeneration that affects roughly 1 in 50,000 males. The disease causes a gradual loss of vision, starting with childhood night blindness, followed by peripheral vision loss and progressing to loss of central vision later in life. Progression continues throughout the individual's life, but both the rate of change and the degree of visual loss are variable among those affected, even within the same family. Kämä, J (1986) “Choroideremia. A clinical and genetic study of 84 Finnish patients and 126 female carriers” Acta Ophthalmologica Supplement. 176: 1-68.

Choroideremia is believed caused by a loss-of-function mutation in the CHM gene which encodes Rab escort protein 1 (REP1), a protein involved in lipid modification of Rab proteins. The lack of a functional protein in the retina has been reported to result in cell death and the gradual deterioration of the retinal pigment epithelium (RPE), photoreceptors and the choroid. Roberts et al. (2002) “Retrospective, longitudinal, and cross sectional study of visual acuity impairment in choroideraemia” The British Journal of Ophthalmology 86(6): 658-62; and Aleman et al. (2016) “Natural History of the Central Structural Abnormalities in Choroideremia: A Prospective Cross-Sectional Study” Ophthalmology 124(3):359-373.

Even though the disease progression can vary significantly, there are general trends. Strunnikova et al., (2012) “Serum biomarkers and trafficking defects in peripheral tissues reflect the severity of retinopathy in three brothers affected by choroideremia” Advances in Experimental Medicine and Biology 723:381-387. The first symptom many individuals with choroideremia notice is a significant loss of night vision, which begins in youth. Peripheral vision loss occurs gradually, starting as a ring of vision loss, and continuing on to “tunnel vision” in adulthood. Khan et al. (2016) “Clinical and Genetic Features of Choroideremia in Childhood” Ophthalmology 123(10):2158-2165; and MacDonald et al. (2015) “Choroideremia” GeneReviews, University of Washington, Seattle.

XI. Nucleic Acid Antisense Oligomer/Suppressor tRNA Delivery Platforms

In one embodiment, the present invention contemplates a method comprising administering a composition comprising a nucleic acid antisense oligomer and a suppressor tRNA. In one embodiment, the administering comprises an adeno-associated virus delivery platform. In one embodiment, the administering comprises a pharmaceutically acceptable composition.

### A. Adeno-Associated Virus Delivery Platforms

Adeno-associated viruses (AAV) are small viruses that infect humans and some other primate species. They belong to the genus Dependoparvovirus, which in turn belongs to the family Parvoviridae. They are small (20 nm) replication-defective, nonenveloped viruses and have linear single-stranded DNA (ssDNA) genome of approximately 4.8 kilobases (kb). Naso et al. (2017) “Adeno-Associated Virus (AAV) as a Vector for Gene Therapy” BioDrugs 31(4):317-334; and Wu et al., (2010) “Effect of Genome Size on AAV Vector Packaging” Molecular Therapy 18(1):80-86.

AAV are not currently known to cause disease and normally result in a very mild immune response. Several additional features make AAV an attractive candidate for creating viral vectors for administering nucleic acid antisense oligomers. AAV vectors incorporating a nucleic acid antisense oligomer can infect both dividing and quiescent cells and persist in an extrachromosomal state without integrating into the genome of the host cell. AAVs apparently lack of pathogenicity. AAVs can also infect non-dividing cells and have the ability to stably integrate into the host cell genome at a specific site (e.g., AAVS1) in the human chromosome 19. Kotin et al., (March 1990) “Site-specific integration by adeno-associated virus” Proceedings of the National Academy of Sciences of the United States of America 87(6):2211-2215; and Surosky et al., (1997) “Adeno-associated virus Rep proteins target DNA sequences to a unique locus in the human genome” Journal of Virology 71(10):7951-7959. This feature makes it somewhat more predictable than retroviruses, which present the threat of a random insertion and of mutagenesis, which is sometimes followed by development of a cancer.

The integration of the AAV genome can be prevented by removal of the rep and cap genes from the vector. The desired nucleic acid antisense oligomer together with a promoter to drive transcription of the gene is inserted between an inverted terminal repeat (ITR) that aid in concatemer formation in the nucleus after the single-stranded vector DNA is converted by host cell DNA polymerase complexes into double-stranded DNA. AAV-based therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Daya et al., (2008) “Gene therapy using adeno-associated virus vectors” Clinical Microbiology Reviews 21(4):583-593.

### B. Pharmaceutically Acceptable Composition Platforms

The present invention further provides pharmaceutical compositions (e.g., comprising the compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

The present invention contemplates several drug delivery systems that provide for roughly uniform distribution, have controllable rates of release. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates a drug delivery system comprising therapeutic agents as described herein.

### Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

### Liposomes

One embodiment of the present invention contemplates liposomes capable of attaching and releasing therapeutic agents described herein. Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one therapeutic agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### Microspheres, Microparticles And Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90 m. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA PGA Copolymer Ratios, J. Biomed. Mater. Res., Vol. I1:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 m and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a therapeutic agent. Preferably, these microparticles are soluble and the selected protein (i.e., fibrinogen or thrombin) creates the walls of the microparticles. Consequently, the therapeutic agents are incorporated within, and between, the protein walls of the microparticle. Heath et al., Microparticles And Their Use in Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

### EXPERIMENTAL

### Example 1

### Cell Culture

### A. Exemplary Cell Lines

CFF-16HBEge CFTR G542X
CFF-16HBEge CFTR Ri162X
CFF-16HBEge CFTR W1282X
CFFT-16HBEge CFTR Y122X

### B. Exemplary Cell Culture Conditions

Medium (Store complete medium at 4° C.)

| Component | CFF Vendor/Catalog | % (final) |
|---|---|---|
| Minimum Essential | Gibco 11095-072 | 89% |
| Medium | | |
| Fetal bovine serum | Gibco 26140-079 | 10% |
| Penicillin/ | Gibco 15140-122 | 1% |
| Streptomycin (100x) | | |

Freezing Solution (Make fresh)

| Component | % (final) |
|---|---|
| Complete media | 40% |
| (see above) | |
| Fetal bovine serum | 50% |
| DMSO | 10% |

### C. Other Reagents

DPBS (Hyclone SH30028.02)
TrypLE Express (Gibco 12604-021)
   D. Coating Solution (Make fresh)

| | | 50 mL final |
|---|---|---|
| Component | CFF Vendor/Catalog | solution |
| LHC-8 basal medium | Gibco 12677-027 | 48 mL |
| Bovine serum albumin | Gibco 15260-037 | 67 μL |
| 7.5% | | |
| Bovine collagen solution, | Advanced BioMatrix | 0.5 mL |
| Type 1 | 5005-100 ML | |
| Fibronectin from human | Thermo Fisher | 0.5 mL |
| plasma, 1 mg/ml | Scientific | |
| | 33016-015 | |

Coat flasks with the coating solution: 1 ml of the solution for a T-25 flask, 2 ml for a T-75. Distribute the solution evenly across the surface, making sure the entire surface is wetted by the solution and leave for 2-3 hours at 37° C. After incubation, thoroughly remove liquid. Do not reuse this solution. Do not rinse the containers. The coated flasks can be stored at 4° C. for several months.

### E. Exemplary Sub-Culturing Protocol

Sub-culture at 80-100% confluency
Remove medium and rinse with DPBS and remove
Add TrypLE Express (spread across the cell surface) and incubate the flask at 37° C.
After 3-5 minutes, examine the flask under a microscope to observe the degree of cell detachment. If the cells are coming off the surface use the palm of your hand and hit against the side of the flask to facilitate further detachment. If the cells are still attached, put the flask back into the incubator for a few more minutes and examine the flask again. The goal is to remove >95% of the cells from the surface.
Pipette the cells repeatedly to break up any clumps and transfer cell suspension into a centrifuge tube.
Spin for 3.5 minutes at 1200 rpm to pellet the cells o Note: If you cannot spin the cells (such as high throughput 96 well format), this step can be skipped
Discard the liquid and resuspend the cell pellet in complete medium.
Split cells at 1:10 to 1:20 for one week passages o CFF media change schedule—3× weekly

### F. Exemplary Liquid Nitrogen Storage Protocol

Treat the cells as for sub-culturing except resuspend the cell pellet in freezing solution.
Transfer the cells into freezing vials, 1-1.5 mL/vial. For a T-75 flask, aliquot into 4-6 vials, a T-75 into 2-4 vials depending on cell density. o CFF freezes 5 million cells/vials for thawing into a T-75 flask.
   Put the vials in controlled cooling chamber and plate in −80° C. freezing overnight (the chamber regulates cooling at ~1° C./minute)
   Move the vials from the cooling chamber into liquid nitrogen storage as quickly as possible

### G. Exemplary Culturing Frozen-Stored Cells

Remove a vial of cells from liquid nitrogen and thaw the cells in a 37° C. water bath
Resuspend with 5 mL complete medium
Spin for 3.5 minutes at 1200 rpm to pellet the cells
Remove liquid (to remove DMSO)
Resuspend cell pellet with 9 mL medium (for T-75 flask) and seed the cells onto a coated flask
Grow cells in 37° C. incubator with 5% CO2.
After overnight incubation, the cells should attach to the flask. The rounded, un-attached cells are dead cells.

### Example II

### Gene Editing

### A. Cas9 Protein Complex

An RNP complex including:
   Cas9: S. pyogenes Cas9
   Guide RNA target sequence: 5′-GAGAAAGACAATATAGTTCT-3′

### B. Protospacer Accessory Motif Sequence: TGG

C. Homology Directed Repair Donor Template Sequence (ssODN: 5′-3′):

GCAAATGCTTGCTAGACCAATAATTAGTTATTCACCTTGCTAA
AGAAATTCTTGCTCGTTGACCTCCACTCAGTGTGATTCCACCT
TCTCAAAGAACTATATTGTCTTTCTCTGCAAACTTGGAGAT

### D. Amplifying/Sequencing Primers

Forward Primer:
5′-ATGGAAGCCCAGTGAAGATAC-3′
Reverse Primer:
5′-CTAGCCATAAAACCCCAGGA-3′

### Example III

### Sequencing

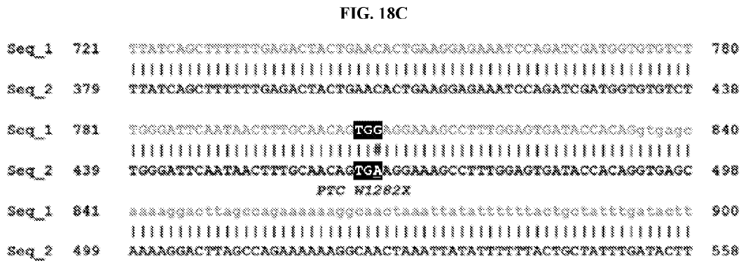

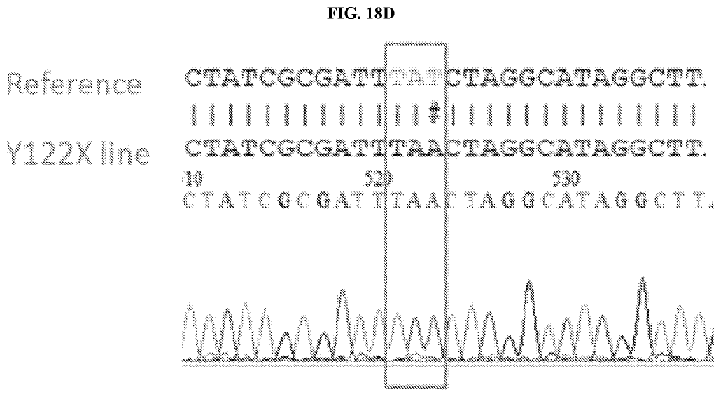

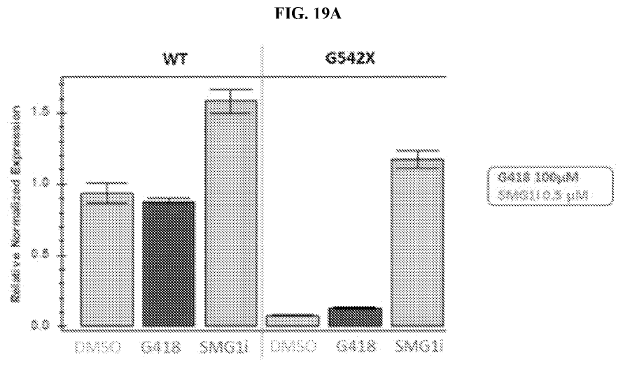

Bulk sequencing of PCR amplicons was performed using the primers in accordance with Example II. A TOPO cloning (e.g, allelic exclusion) method sequenced the plasmid vector. See, FIG. 18A-FIG. 18D.

The data shows that no large deletions or re-arrangements were identified with next generation sequencing of the CFTR locus. However, an insertion of unknown size was observed at position (hg38) Chr7:117536118 in intron 6 of one CFTR allele in the 16HBE14o− parental cells and the 16HBEge cells that were derived from them. This insertion contains SV40 genomic sequence, which was used in the immortalization process to create the 16HBE14o− cells and is not a result of gene editing. Several lines of evidence support that the allele carrying the insertion yields a degraded CFTR transcript or non-functional CFTR; therefore, the 16HBE14o− cells are functionally mono-allelic. The 16HBEge cell lines are homozygous for the engineered CFTR variant and express CFTR from the same number of alleles as the 16HBE14o− cells.

### Example IV

### Gene Expression

The example presents the results of mRNA analysis by quantitative polymerase chain reaction and (qPCR) and protein analysis by Western Blot.

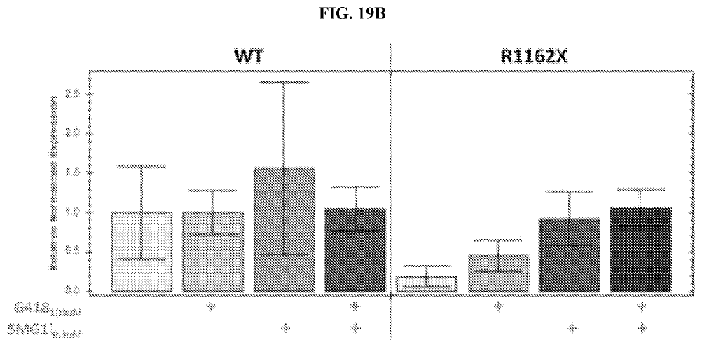

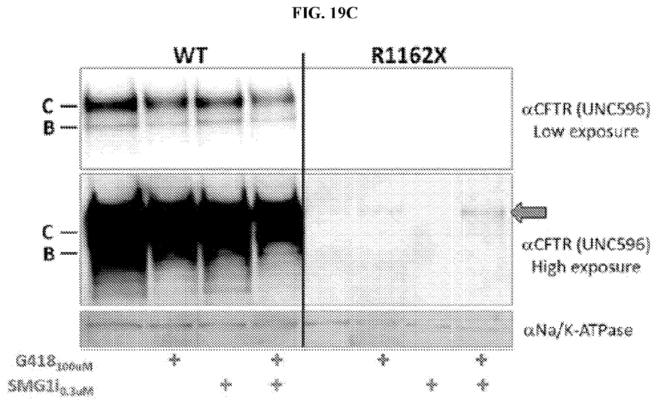

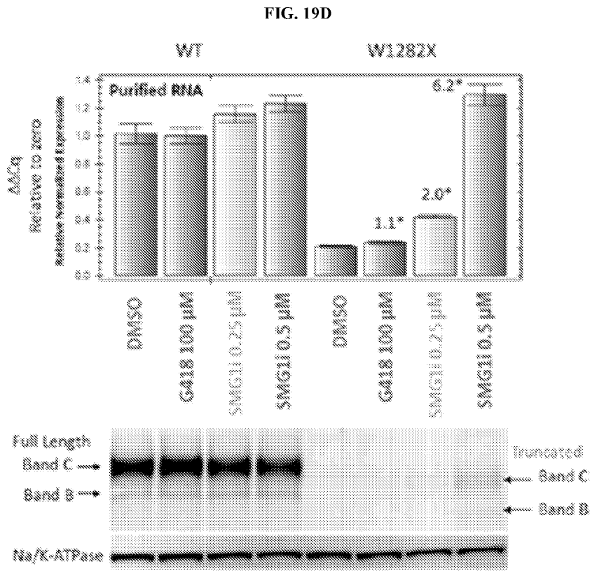

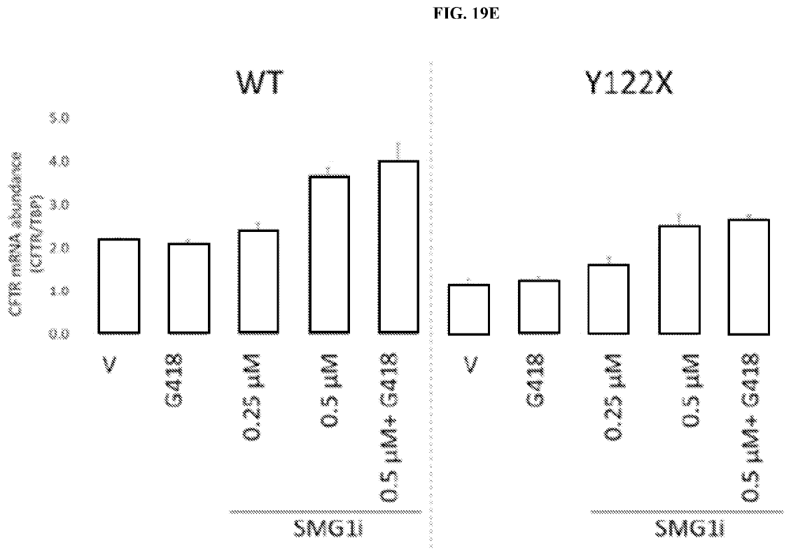

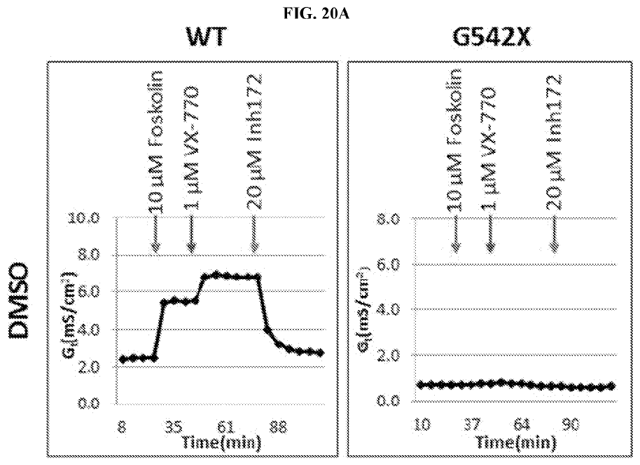

The data shows that the antisense, SMG1i, is a pharmacological inhibitor of the NMD-mediator SMG1 and restores mRNA levels of the PTC alleles. As usual, a smaller increase in CFTR WT mRNA is also observed with SMG1i. See, FIG. 19A-F.

### Example V

### Electrophysiology

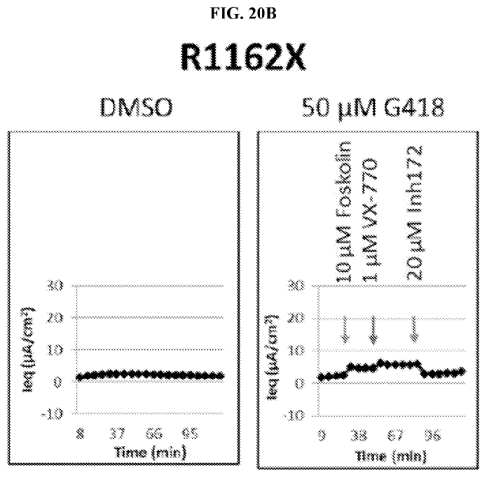

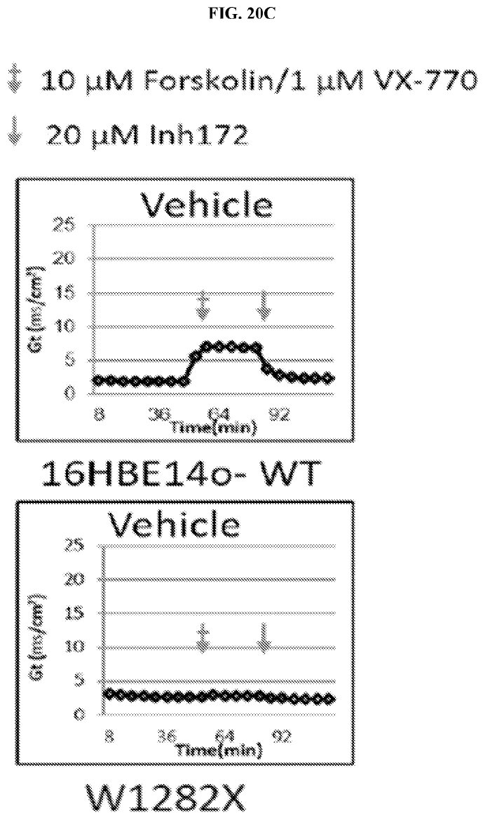

A functional assay was performed at one (1) week post-filter seeding using a TECC/24 Conductance Assay that demonstrated resistance but no measurable CFTR function. See, FIG. 20A- FIG. 20C.

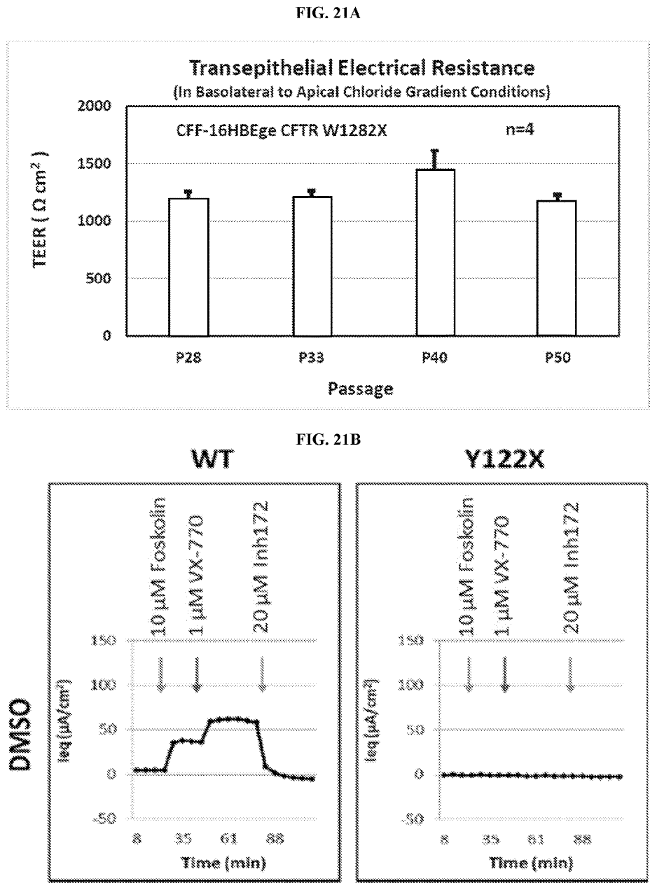

The transepithelial resistance of the CFF-16HBEge lines is stable over time (up to passage 50 tested in the CFF-16HBEge CFTR W1282X cell line). See, FIG. 21A- FIG. 21B.

### Example VI

### Oligonucleotides

Oligonucleotides were ordered from a commerically available source (IDT) or synthesized as previously described. (Hariharan et al. 2023).

### Example VII

### Preparation of mRNAs and ser-tRNA^{UGA}

mRNAs

DNA constructs coding for model mRNAs were ordered from Genewiz (Azenta) (for the principal scheme of the constructs, see FIG. 2A). Point mutations were introduced with the Q5® Site-Directed Mutagenesis Kit (NEB) or through PCR and overlapping primers. All PCR reactions were performed with Phusion® High-Fidelity DNA Polymerase (NEB). The constructs were sequenced in Genewiz (Azenta).

To generate DNA-templates for in vitro transcription, the constructs were subjected to PCR with a pair of primers annealing to globin UTRs: the forward primer

(IDT)
5′-tttttTAATACGACTCACTATAGACACTTGCTTTTGACA
CAACTGTG-3′

containing a T7-promoter (underscored) and the reverse primer 5′-ttttttttttttttttttttttttttttttGCAATGAAAATAAATTTCCTTTATTAGCC-3′ (IDT) coding for a 30-nt poly-A-stretch.

The PCR reactions were fractionated with phenol:chloroform (pH=8.0), PCR-templates were precipitated from the water-soluble fraction with 0.3 M NaOAc and ethanol. The pellets were washed with cold 80% ethanol, dissolved in mQ (deionized) water and used in in vitro transcription.

In vitro transcription reactions were prepared by mixing the dissolved template, 4 mM each rNTP, 5% homemade T7-polymerase (Susorov et al. 2020), TBx1 (166 mM Hepes-KOH, pH=7.5, 20 mM Mg(OAc)₂, 40 mM DTT, 2 mM spermidine, 100 ug/ul BSA (NEB)), followed by incubation at 37° C. for 3 h. After the incubation, the insoluble PPi was removed by centrifugation and RNA was precipitated from the supernatant with 2.5 M LiCl. The pellet was washed with cold 80% ethanol and dissolved in mQ water.

To test whether mRNA capping affects the R-ASO-induced readthrough, we compared capped and uncapped mRNA translation in RRL. To this end, the mRNAs were capped using Vaccinia Capping System (NEB); 30nt polyA tails were further elongated by E. coli Poly(A) Polymerase (NEB) according to the manufacturer protocol.

In vitro RNAse H assays were performed with E. coli RNAse H (NEB) according to the manufacturer's protocol. Integrity of mRNAs was assessed by electrophoresis in 1% TBE-agarose, the concertation was determined with Nanodrop (Thermo Scientific).

### Ser-tRNA^{UGA}

The sequence for the human tRNA^{Ser} was retrieved from tRNA-database (tRNA-Ser-CGA-1-1. gtrnadb.ucsc.edu/index. The anticodon sequence CGA was changed to UCA(to match UGA stop-codon). The construct was ordered from Genewiz (Azenta).

To generate PCR-template for in vitro transcription, the following primers were used: the forward

(IDT)
5′-tttttTAATACGACTCACTATAGCTGTGATGGC-3′

containing T7-promoter (underscored) and the reverse 5′-mTmGGCGCTGTGAGCAGGATTCG-3′ (IDT), containing 2′-OME modified nucleotides (underscored) to preclude T7-polymerase from adding non-template nucleotides to the conservative CCA-end of tRNA (Kao et al. 1999; Katoh and Suga 2019).

The PCR-template was used in in vitro transcription as described above, transcription reaction was fractionated with phenol:chloroform, pH=4.5, the water-soluble fraction was precipitated with 0.3 M NaOAc and ethanol. The pellet was washed with cold 80% ethanol and dissolved in mQ water; free nucleotides were removed through repetitive concentrating with Amicon 10 kDa cutoff centrifugal filters (MilliporeSigma). The concentration of tRNA^{UGA} was determined with Nanodrop (Thermo Scientific).

To perform aminoacylation, 2.5 uM tRNA^{UGA} was mixed with 6 mM ATP, 0.05 mM serine, 5% of yeast S100 extract, 1 mM DTT and ABx1 (50 mM Hepes-KOH, pH=7.5, 30 mM KCl, 50 mM MgCl₂, 30 uM MnCl₂, 30 uM ZnCl₂). Yeast S100 extract was prepared as described in (Eyler and Green 2011). The reaction mixture was incubated in thermocycler (Bio-Rad) at 25° C. for 25 hours. After the incubation, the sample was processed the same way as after in vitro transcription and the extent of aminoacylation was assessed using 7% PAGE in 8M Urea. The gel was stained with 0.2% methylene blue and imaged with ChemiDoc (Bio-Rad).

### Example VIII

### In Vitro Translation

In vitro translation was performed as described before (Susorov et al. 2020). Reaction mixture containing 50% of nuclease-treated rabbit reticulocyte lysate (RRL) (Promega) was supplemented with 30 mM Hepes-KOH (pH=7.5), 50 mM KOAc, 1.0 mM MgOAc₂, 0.2 mM ATP and GTP, 0.04 mM of 20 aminoacids (Promega), and 2 mM DTT. Nanoluciferase substrate furimazine (Promega) was added to the mixture to 1% concentration. 9 μl aliquots of the mixture were placed in 384-well plate (Corning Low Volume White Round Bottom), mixed with water/oligos/G418/ser-tRNA^{UGA} (final concetrations: na/10 uM/0.5 uM/0.5 uM correspondingly, unless otherwise stated) and incubated at 30° C. for 5 min in microplate reader (Tecan INFINITE M1000 PRO or Tecan Spark). Translation reactions were started by the addition of mRNAs to 30 nM final concentration followed by recording luminescence signal in the kinetic mode. After 20 min, reactions were stopped by transferring to 100 ul of TRIzol reagent (Invitrogen) followed by fractionation into total protein and total RNA fractions according to the manufacturer instructions.

The changes of the recorded luminescence signal over time points were used to derive observed rates of translation. The maximal values of the rates were used in comparisons between different mRNAs/reaction conditions. The calculations were performed in Microsoft Excel and plotted in GraphPad Prizm (www.graphpad.com).

### Example IX

### Western Blot of the Total Protein Fraction

The pellets of total protein obtained in TRIzol fractionation were dissolved in 20 ul of 80 mM Tris-HCl, pH 6.8, 2% SDS, 20% glycerol, 10% Beta-ME, 8M Urea by repeated heating at 95° C. for 10 min and vortexing. The dissolved samples were applied to discontinuous PAGE with 5% concentrating and 12% resolving gels, both containing 8M Urea. After the electrophoresis, proteins were transferred to PVDF membrane using semi-dry machine (BioRad). The membranes were blocked with 5% dry milk (ChemCruz) in PBST for 1 h at RT and incubated with the primary monoclonal antibody (1:500) against the N-terminal twin-strep tag (GT517, Invitrogen) overnight at 4° C. The membranes were washed and incubated with the secondary HRP antibody (1:2000, Goat anti-Mouse, Invitrogen) for 1 h at RT then washed and imaged with SuperSignal West Atto kit (Thermo Scientific) and ChemiDoc (BioRad) using default exposure determined by the device.

### Example X

### mRNA Detection in the Total RNA Fraction

To assess integrity of the model mRNAs after translation we developed an approach based on agarose electrophoresis in the presence of specific mRNA probes. The total RNA pellets obtained in TRIzol fractionation were dissolved in 40 mM PIPES, pH 6.8, 100 mM NaCl, 1 mM EDTA, 90% deionized formamide and 1 uM of each three FAM-labeled DNA oligonucleotides (IDT) annealing over CDS of the model mRNA (1 annealing in CFTR-part, 2 in nanoluciferase part). The samples were heated for 2 min at 95° C. and placed on ice, then mixed with the loading buffer (40 mM PIPES, pH 6.8, 6% glycerol, 5 mM EDTA, 12.5% deionized formamide, 0.025% Bromophenol Blue) and subjected to electrophoresis in 2%-TBE agarose. After the electrophoresis, the gels were cut just above bromophenol band (to remove non-bound FAM-labeled probes) and imaged in epi/fluorescein mode in ChemiDoc (BioRad). The gels then were stained with SYBR Safe DNA stain (Invitrogen) to visualize the total RNA and imaged in ChemiDoc (BioRad).

### Example XI

### Termi-Luc Assay

The Termi-luc assay was performed as described previously (Susorov et al. 2020) with modifications. In short, 1 ml translation reaction was assembled as described above and supplemented with 1.2 uM of the mutant human eRF1IAA (instead of eRF1^{AGQ}) followed by incubation at RT for 5 min. Then 24 μg of the model mRNA was added and the reaction mixture was incubated for 10 min at 30° C. Translation was stopped by adjusting MgOAc₂ to 5 mM and KOAc to 300 mM. The reaction was subjected to centrifugation in 10-35% sucrose gradient (using Beckman Coulter ultracentrifuge, SW41 Ti rotor), and fractionated using gradient master (Biocomp). The A₂₅₄ pick corresponding to 80S was collected and concentrated to 3.5 A₂₆₀U/ul using Amicon 50 kDa cutoff filters (MilliporeSigma) with the buffer 50 mM Hepes-KOH, pH 7.5, 100 mM KCl, 5 mM MgOAc₂, 5% glycerol, 2 mM DTT.

10 ul aliquots of ribosomal complexes were premixed with the buffer DBx1 50 mM Tris-HCl, pH 7.5, 100 mM KCl, 2.5 mM MgOAc₂, 0.01% Triton X-100 (concentration in the final 20 ul sample) to get 12 reaction mixtures with the volume of 14 ul. The important modification here in comparison with the previous procedure (Susorov et al. 2020) was inclusion detergent (Triron X-100) into the reaction mixture which allowed to significantly increase both reproducibility and signal level of the assay. Inclusion of Tween 20 or BSA demonstrated a similar improvement. The aliquots were applied to the 384-well plate (Corning Low Volume White Round Bottom), mixed with oligonucleotides (to 10 uM) or mQ water, and incubated in the multiplate reader for 5 min at 30° C. Furimazine (Promega) was added to 1% and the baseline signal was recorded for 2 min. Then yeast eRF1*eRF3*GTP complex was added (preincubated in the buffer DBx1, supplemented with 0.2 mM GTP) to the final concentration of proteins 0.05 uM and luciferase signal was recorded in the kinetic mode.

The raw curves were fit with exponential plateau equation in GraphPad Prizm and k_{obs} were plotted in the software.

### Example XII

### Bioinformatics

To generate semi-random sequences for +8-+27 site from a redundant RY-template a Bash shell script was written, utilizing random choice of A or G for R and T or C for Y. Three such sequences were ordered from Genewiz (Azenta).

To isolate +8-+27 contexts in the reported human nonsense mutation sites, the list of affected transcripts was retrieved from LOVD3 database (databases.lovd.nl/shared/genes) along with the positions of the premature stop codons. For the simplicity, premature stop codons arising from a frameshift were excluded. The transcripts CDS were retrieved using NCBI Batch Entrez (nebi.nlm.nih.gov/sites/batchentrez) as FASTAs. The FASTAs and premature stops positions were used to create a CSV database of premature stops contexts with the help of a Bash shell script. The database was queried using mixtures of awk/grep commands.

### Example XIII

### Software

All calculations and data visualization were done in Microsoft Excel and GraphPad Prism (www.graphpad.com). The figures were prepared in Adobe Illustrator. The pictures of ribosome were prepared with the help of ChimeraX (Goddard et al. 2018; Pettersen et al. 2021) and Adobe Illustrator using (PDB:6MTB) model of the rabbit 80S ribosome. Schematic images of tRNAs were prepared with Forna at RNAfold server (rna.tbi.univie.ac.at//cgi-bin/RNAWebSuite/-RNAfold.cgi). Graphic alignment of +8-+27 contexts was performed using WebLogo (weblogo.berkeley.edu/logo.cgi). The pictures of the modified nucleotides were prepared in ChemSketch (acdlabs.com/products/chemsketch/).

### REFERENCES

Eyler D E, Green R. 2011. Distinct response of yeast ribosomes to a miscoding event during translation. RNA.
Goddard T D, Huang C C, Meng E C, Pettersen E F, Couch G S, Morris J H, Ferrin T E. 2018. UCSF ChimeraX: Meeting modern challenges in visualization and analysis. Protein Sci.
Hariharan V N, Shin M, Chang C W, O'Reilly D, Biscans A, Yamada K, Guo Z, Somasundaran M, Tang Q, Monopoli K, et al. 2023. Divalent siRNAs are bioavailable in the lung and efficiently block SARS-CoV-2 infection. Proc Natl Acad Sci USA.
Kao C, Zheng M, Rüdisser S. 1999. A simple and efficient method to reduce nontemplated nucleotide addition at the 3′ terminus of RNAs transcribed by T7 RNA polymerase. RNA.
Katoh T, Suga H. 2019. Flexizyme-catalyzed synthesis of 3′-aminoacyl-NH-tRNAs. Nucleic Acids Res 47.
Pettersen E F, Goddard T D, Huang C C, Meng E C, Couch G S, Croll T I, Morris J H, Ferrin T E. 2021. UCSF ChimeraX: Structure visualization for researchers, educators, and developers. Protein Sci.
Susorov D, Egri S, Korostelev A A. 2020. Termi-Luc: A versatile assay to monitor full-protein release from ribosomes. Rna 26: 2044-2050.

1. A method, comprising:
a) providing;
i) a patient comprising a messenger ribonucleic acid (mRNA) molecule having a nonsense stop codon and exhibits at least one symptom of a genetic disorder or disease; and
ii) a pharmaceutically acceptable composition comprising a nucleic acid antisense oligomer that is at least partially complementary to said mRNA starting between a +4-+9 nucleotide position downstream of the first nucleotide of said nonsense stop codon; and
b) administering said pharmaceutically acceptable composition to said patient such that said at least one symptom of said genetic disorder or disease is reduced,
wherein said nucleic acid antisense oligomer is selected from at least one of the group consisting of SEQ ID NOs 712-730.

2. The method of claim 1, wherein said pharmaceutical composition further comprises a suppressor tRNA (stRNA) that is complementary to said nonsense stop codon.

3. The method of claim 1, wherein said stRNA is aminoacylated.

4. The method of claim 1, wherein said stRNA is ser-tRNA^{UGA}.

5-14. (canceled)

15. The method of claim 1, wherein said nucleic acid antisense oligomer comprises at least one modified nucleotide.

16. The method of claim 15, wherein said modified nucleotide is selected from at least one of the group consisting of a phosphate (P) linkage, a phosphothioate (PS) linkage a methylphosphonate, a phosphorodiamidate morpholino (PMO), a 2′-H (dN), a 2′-hydroxy (rN), a 2′-fluoride (F), 2′-locked (IN), a 2′-O-methyl (Ome), a 2′-methoxyethyl (Moe), and or a mismatched nucleotide.

17. (canceled)

18. The method of claim 1, wherein said genetic disorder or disease is caused by a truncated expression of said protein.

19. (canceled)

20. The method of claim 1, wherein said genetic disorder or disease is selected from at least one of the group consisting of Duchenne muscular dystrophy, non-spherocytic hemolytic anemia, recessive retinitis pigmentosa, breast cancer, ovarian cancer, bull's-eye maculopathy, neurofibromatosis type 1, tuberous sclerosis complex 1, tuberous sclerosis complex 2, recessive retinitis pigmentosa, progressive pseudorheumatoid dysplasia, epilepsy, and hemophilia.

21. The method of claim 1, wherein said pharmaceutically acceptable composition further comprises an aminoglycoside.

22. (canceled)

23. The method of claim 21, wherein said aminoglycoside is G418.

24. The method of claim 21, wherein said aminoglycoside is selected from at least one of the group consisting of gentamicin, amikacin, tobramycin, kanamycin, streptomycin, and neomycin.

25. The method of claim 1, wherein said nonsense stop codon comprises at least one sequence selected from the group consisting of UAA, UAG, and UGA.

26. The method of claim 1, wherein said nonsense stop codon is at least one of the group consisting of UGAC, UGAG, and UGAU.

27. The method of claim 1, wherein said nucleic acid antisense oligomer is single stranded.

28. The method of claim 1, wherein said nucleic acid antisense oligomer is double stranded.

29. The method of claim 1, wherein said pharmaceutically acceptable composition comprises an adeno-associated virus.

30. The method of claim 1, wherein said pharmaceutically acceptable composition is selected from at least one of the group consisting of a microparticle, a nanoparticle, and a liposome.

31. The method of claim 1, wherein said pharmaceutically acceptable composition is selected from at least one of the group consisting of a tablet, a capsule, and a gel.

32-118. (canceled)
